SEQUENCE LISTING (79 sequences)

SEQ ID NO: 1 - RNA, 50 nt, synthetic construct
  source 1..50: mol_type = other RNA
tggtgggcggtgttgtcatagcgacagtgatcgtcatcaccttggtgatg

SEQ ID NO: 2 - RNA, 25 nt, synthetic construct
  source 1..25: mol_type = other RNA
atcactgtcgccatgacaacaccgc

SEQ ID NO: 3 - DNA, 20 bp, synthetic construct
  source 1..20: mol_type = other DNA
ctcgacgcaagccataacac

SEQ ID NO: 4 - DNA, 20 bp, synthetic construct
  source 1..20: mol_type = other DNA
tggaccgactggaaacgtag

SEQ ID NO: 5 - DNA, 17 bp, synthetic construct
  source 1..17: mol_type = other DNA
cattggactcatggtgg

SEQ ID NO: 6 - DNA, 16 bp, synthetic construct
  source 1..16: mol_type = other DNA
cagcatcaccaaggtg

SEQ ID NO: 7 - DNA, 18 bp, synthetic construct
  source 1..18: mol_type = other DNA
  modified_base order(5,10..12): mod_base = OTHER; note = Locked nucleic acids
tgttgtcatagcgacagt

SEQ ID NO: 8 - DNA, 18 bp, synthetic construct
  source 1..18: mol_type = other DNA
  modified_base 10..11: mod_base = OTHER; note = Locked nucleic acids
tgttgtcatggcgacagt

SEQ ID NO: 9 - RNA, 64 nt, Homo sapiens
  source 1..64: mol_type = other RNA
gaggactcctctgtctttgcccagagcatcccgtggaacctggagcggattacccctccacggt

SEQ ID NO: 10 - RNA, 64 nt, Macaca mulatta
  source 1..64: mol_type = other RNA
gaggactcctctgtcttcgcccagagcatcccatggaacctggagcgaattactcctgcacggt

SEQ ID NO: 11 - RNA, 38 nt, synthetic construct
  source 1..38: mol_type = other RNA
  modified_base 26: mod_base = OTHER; note = Deoxyinosine
  modified_base order(1..6,15..19,27,30..38): mod_base = OTHER; note = 2-Prime-OMe nucleotides
  modified_base order(7..9,14,21): mod_base = OTHER; note = 2-Prime-MOE nucleotides
  modified_base order(10..13,20,22..25,29): mod_base = OTHER; note = 2-Prime-MOE modified 5-methyl-nucleotides
  modified_base 28: mod_base = OTHER; note = 2-Prime-F nucleotide
  modified_base order(1..7,15..20,25..26,28..38): mod_base = OTHER; note = Phosphorothioate linkage
  modified_base 26^27: mod_base = OTHER; note = Methylphosphonate linkage
cgctccaggttccatgggatgctccnggcgaagacaga

SEQ ID NO: 12 - RNA, 34 nt, synthetic construct
  source 1..34: mol_type = other RNA
  modified_base 22: mod_base = OTHER; note = Deoxyinosine
  modified_base order(1..2,11..15,23,26..34): mod_base = OTHER; note = 2-Prime-OMe nucleotides
  modified_base order(3..5,10,17): mod_base = OTHER; note = 2-Prime-MOE nucleotides
  modified_base order(6..9,16,18..21,25): mod_base = OTHER; note = 2-Prime-MOE modified 5-methyl-nucleotides
  modified_base 24: mod_base = OTHER; note = 2-Prime-F nucleotide
  modified_base 22^23: mod_base = OTHER; note = Methylphosphonate linkage
  modified_base order(1..3,11..16,21..22,24..34): mod_base = OTHER; note = Methylphosphonate linkage
ccaggttccatgggatgctccnggcgaagacaga

SEQ ID NO: 13 - RNA, 30 nt, synthetic construct
  source 1..30: mol_type = other RNA
  modified_base 18: mod_base = OTHER; note = Deoxyinosine
  modified_base 20: mod_base = OTHER; note = 2-Prime-F nucleotide
  modified_base order(7..11,19,22..30): mod_base = OTHER; note = 2-Prime-OMe nucleotides
  modified_base order(12,14..17,21): mod_base = OTHER; note = 2-Prime-MOE modified 5-methyl-nucleotides
  modified_base 13: mod_base = OTHER; note = 2-Prime-MOE nucleotide
  modified_base order(7..12,17..18,20..30): mod_base = OTHER; note = Phosphorothioate linkage
  modified_base 18^19: mod_base = OTHER; note = Methylphosphonate linkage
gttccatgggatgctccnggcgaagacaga

SEQ ID NO: 14 - RNA, 25 nt, synthetic construct
  source 1..25: mol_type = other RNA
  modified_base 13: mod_base = OTHER; note = Deoxyinosine
  modified_base 15: mod_base = OTHER; note = 2-Prime-F nucleotide
  modified_base order(1..6,14,17..25): mod_base = OTHER; note = 2-Prime-OMe nucleotides
  modified_base order(7,9..12,16): mod_base = OTHER; note = 2-Prime-MOE modified 5-methyl-nucleotides
  modified_base 8: mod_base = OTHER; note = 2-Prime-MOE nucleotide
  modified_base 15: mod_base = OTHER; note = 2-Prime-F nucleotide
  modified_base order(1..7,12..13,15..25): mod_base = OTHER; note = Phosphorothioate linkage
  modified_base 13^14: mod_base = OTHER; note = Methylphosphonate linkage
atgggatgctccnggcgaagacaga

SEQ ID NO: 15 - RNA, 23 nt, synthetic construct
  source 1..23: mol_type = other RNA
  modified_base 11: mod_base = OTHER; note = Deoxyinosine
  modified_base 13: mod_base = OTHER; note = 2-Prime-F nucleotide
  modified_base order(1..4,12,15..23): mod_base = OTHER; note = 2-Prime-OMe nucleotides
  modified_base order(5,7..10,14): mod_base = OTHER; note = 2-Prime-MOE modified 5-methyl-nucleotides
  modified_base 6: mod_base = OTHER; note = 2-Prime-MOE nucleotide
  modified_base order(1..5,10..11,13..23): mod_base = OTHER; note = Phosphorothioate linkage
  modified_base 11^12: mod_base = OTHER; note = Methylphosphonate linkage
gggatgctccnggcgaagacaga

SEQ ID NO: 16 - RNA, 25 nt, synthetic construct
  source 1..25: mol_type = other RNA
  modified_base 13: mod_base = OTHER; note = Deoxyinosine
  modified_base 15: mod_base = OTHER; note = 2-Prime-F nucleotide
  modified_base order(1..10,14,17..25): mod_base = OTHER; note = 2-Prime-OMe nucleotides
  modified_base order(11..12,16): mod_base = OTHER; note = 2-Prime-MOE modified 5-methyl-nucleotides
  modified_base 13^14: mod_base = OTHER; note = Methylphosphonate linkage
  modified_base order(1..13,14..25): mod_base = OTHER; note = Phosphorothioate linkage
atgggatgctccnggcgaagacaga

SEQ ID NO: 17 - DNA, 20 bp, synthetic construct
  source 1..20: mol_type = other DNA
aagttgccccatgtcgacta

SEQ ID NO: 18 - DNA, 24 bp, synthetic construct
  source 1..24: mol_type = other DNA
tcactctgtatgctggtgtctagg

SEQ ID NO: 19 - DNA, 15 bp, synthetic construct
  source 1..15: mol_type = other DNA
ccgtggaggggtaat

SEQ ID NO: 20 - DNA, 22 bp, synthetic construct
  source 1..22: mol_type = other DNA
tgggttgacaaatatcaagacg

SEQ ID NO: 21 - DNA, 24 bp, synthetic construct
  source 1..24: mol_type = other DNA
caccatgatgaatggatgtgtact

SEQ ID NO: 22 - DNA, 17 bp, synthetic construct
  source 1..17: mol_type = other DNA
gcaatcattggactcat

SEQ ID NO: 23 - DNA, 36 bp, synthetic construct
  source 1..36: mol_type = other DNA
ggtgggcggtgttgtcatrgcgacagtgatcgtcat

SEQ ID NO: 24 - DNA, 26 bp, synthetic construct
  source 1..26: mol_type = other DNA
tgtgcgtgtgtcactagcgagcagtg

SEQ ID NO: 25 - RNA, 58 nt, Mus musculus
  source 1..58: mol_type = other RNA
ctgttggatggagaacaactctaggcagaggtctcaaaggctggggctgtgttggaca

SEQ ID NO: 26 - RNA, 38 nt, Mus musculus
  source 1..38: mol_type = other RNA
gccccagcctttgagacctctgcccagtgttgttctcc

SEQ ID NO: 27 - DNA, 36 bp, synthetic construct
  source 1..36: mol_type = other DNA
  misc_feature order(1..24,27..36): note = RNA
  misc_feature 25..26: note = DNA
  modified_base order(1..6,15..19,27,30..36): mod_base = OTHER; note = 2-Prime-OMe nucleotides
  modified_base order(7,13..14,22,29): mod_base = OTHER; note = 2-Prime-MOE nucleotides
  modified_base order(8..12,20..21,23..24): mod_base = OTHER; note = 2-Prime-MOE modified 5-methyl-nucleotides
  modified_base 28: mod_base = OTHER; note = 2-Prime-F nucleotide
  modified_base order(1..7,15..20,31..36): mod_base = OTHER; note = Phosphorothioate linkage
  modified_base 26^27: mod_base = OTHER; note = Methylphosphonate linkage
gccccagcctttgagacctctgtccagagttgttct

SEQ ID NO: 28 - DNA, 23 bp, synthetic construct
  source 1..23: mol_type = other DNA
  misc_feature order(1..11,14..23): note = RNA
  misc_feature 12..13: note = DNA
  modified_base order(1..6,14,17..23): mod_base = OTHER; note = 2-Prime-OMe nucleotides
  modified_base order(7..8,10..11): mod_base = OTHER; note = 2-Prime-MOE modified 5-methyl-nucleotides
  modified_base order(9,16): mod_base = OTHER; note = 2-Prime-MOE nucleotides
  modified_base 15: mod_base = OTHER; note = 2-Prime-F nucleotide
  modified_base order(1..7,18..23): mod_base = OTHER; note = Phosphorothioate linkage
  modified_base 13^14: mod_base = OTHER; note = Methylphosphonate linkage
agacctctgtccagagttgttct

SEQ ID NO: 29 - DNA, 27 bp, synthetic construct
  source 1..27: mol_type = other DNA
  misc_feature order(1..13,16..27): note = RNA
  misc_feature 14..15: note = DNA
  modified_base order(1..12,16,19..27): mod_base = OTHER; note = 2-Prime-OMe nucleotides
  modified_base 13: mod_base = OTHER; note = 2-Prime-MOE modified 5-methyl-nucleotide
  modified_base 19: mod_base = OTHER; note = 2-Prime-MOE nucleotide
  modified_base 18: mod_base = OTHER; note = 2-Prime-F nucleotide
  modified_base order(1..15,16..27): mod_base = OTHER; note = Phosphorothioate linkage
  modified_base 15^16: mod_base = OTHER; note = Methylphosphonate linkage
tgagacctctgtccagagttgttctcc

SEQ ID NO: 30 - DNA, 23 bp, synthetic construct
  source 1..23: mol_type = other DNA
agtactcacagtcatggggctca

SEQ ID NO: 31 - DNA, 22 bp, synthetic construct
  source 1..22: mol_type = other DNA
gccaggacacccactgtatgat

SEQ ID NO: 32 - DNA, 16 bp, synthetic construct
  source 1..16: mol_type = other DNA
tggggctcatggccct

SEQ ID NO: 33 - DNA, 35 bp, synthetic construct
  source 1..35: mol_type = other DNA
ggatggagaacaactctrggcagaggtctcaaagg

SEQ ID NO: 34 - DNA, 38 bp, synthetic construct
  source 1..38: mol_type = other DNA
cgtgatgagacactcgtagcagagtctgcagagctgca

SEQ ID NO: 35 - DNA, 38 bp, synthetic construct
  source 1..38: mol_type = other DNA
ccgctccaggttccacgggatgctcygggcaaagacag

SEQ ID NO: 36 - DNA, 30 bp, synthetic construct
  source 1..30: mol_type = other DNA
tcgctcagtcacgatgcgtctgcagactag

SEQ ID NO: 37 - DNA, 29 bp, synthetic construct
  source 1..29: mol_type = other DNA
  misc_feature order(1..15,17..29): note = RNA
  misc_feature 16: note = DNA
  modified_base 16: mod_base = OTHER; note = DNA nucleotide carrying a Benner base
  modified_base 17: mod_base = OTHER; note = Deoxyinosine
  modified_base 17^18: mod_base = OTHER; note = Methylphosphonate linkage
  modified_base order(1..4,15,20): mod_base = OTHER; note = 2-Prime-MOE modified 5-methyl-nucleotides
  modified_base 5: mod_base = OTHER; note = 2-Prime-MOE nucleotide
  modified_base order(6..9,11,13,18,22,24,26..29): mod_base = OTHER; note = 2-Prime-OMe nucleotides
  modified_base order(10,12,14,19,21,23,25): mod_base = OTHER; note = 2-Prime-F nucleotides
  modified_base order(6..15,16..17,19..28): mod_base = OTHER; note = Phosphorothioate linkage
  modified_base order(1..2,28..29): mod_base = OTHER; note = Phosphoramidate linkage
ttccacgggatgctcnnggcaaagacaga

SEQ ID NO: 38 - DNA, 27 bp, synthetic construct
  source 1..27: mol_type = other DNA
  misc_feature order(1..13,15..27): note = RNA
  misc_feature 14: note = DNA
  modified_base 14: mod_base = OTHER; note = DNA nucleotide carrying a Benner base
  modified_base 15: mod_base = OTHER; note = Deoxyinosine
  modified_base order(1..2,13,18): mod_base = OTHER; note = 2-Prime-MOE modified 5-methyl-nucleotides
  modified_base 3: mod_base = OTHER; note = 2-Prime-MOE nucleotide
  modified_base order(4..7,9,11,16,20,22,24..27): mod_base = OTHER; note = 2-Prime-OMe nucleotides
  modified_base order(8,10,12,17,19,21,23): mod_base = OTHER; note = 2-Prime-F nucleotides
  modified_base order(4..13,14..15,17..26): mod_base = OTHER; note = Phosphorothioate linkage
  modified_base 15^16: mod_base = OTHER; note = Methylphosphonate linkage
  modified_base order(1..2,26..27): mod_base = OTHER; note = Phosphoramidate linkage
ccacgggatgctcnnggcaaagacaga

SEQ ID NO: 39 - DNA, 25 bp, synthetic construct
  source 1..25: mol_type = other DNA
  misc_feature order(1..11,13..25): note = RNA
  misc_feature 12: note = DNA
  modified_base 12: mod_base = OTHER; note = DNA nucleotide carrying a Benner base
  modified_base 13: mod_base = OTHER; note = Deoxyinosine
  modified_base 1: mod_base = OTHER; note = 2-Prime-MOE nucleotide
  modified_base order(2..5,7,9,14,18,20,22..25): mod_base = OTHER; note = 2-Prime-OMe nucleotides
  modified_base order(6,8,10,15,17,19,21): mod_base = OTHER; note = 2-Prime-F nucleotides
  modified_base order(1..2,24..25): mod_base = OTHER; note = Phosphoramidate linkage
  modified_base order(2..11,12..13,15..24): mod_base = OTHER; note = Phosphorothioate linkage
  modified_base 13^14: mod_base = OTHER; note = Methylphosphonate linkage
  modified_base order(11,16): mod_base = OTHER; note = 2-Prime-MOE modified 5-methyl-nucleotides
acgggatgctcnnggcaaagacaga

SEQ ID NO: 40 - DNA, 27 bp, synthetic construct
  source 1..27: mol_type = other DNA
  misc_feature order(1..13,15..27): note = RNA
  misc_feature 14: note = DNA
  modified_base 14: mod_base = OTHER; note = DNA nucleotide carrying a Benner base
  modified_base 15: mod_base = OTHER; note = Deoxyinosine
  modified_base order(1..2,13,18): mod_base = OTHER; note = 2-Prime-MOE modified 5-methyl-nucleotides
  modified_base 3: mod_base = OTHER; note = 2-Prime-MOE nucleotide
  modified_base order(4..6,16,25..27): mod_base = OTHER; note = 2-Prime-OMe nucleotides
  modified_base order(7..12,17,19..24): mod_base = OTHER; note = 2-Prime-F nucleotides
  modified_base order(1..2,26..27): mod_base = OTHER; note = Phosphoramidate linkage
  modified_base order(4..13,14..15,17..26): mod_base = OTHER; note = Phosphorothioate linkage
  modified_base 15^16: mod_base = OTHER; note = Methylphosphonate linkage
ccatgggatgctcnnggcgaagacaga

SEQ ID NO: 41 - DNA, 30 bp, synthetic construct
  source 1..30: mol_type = other DNA
  modified_base 26: mod_base = OTHER; note = Deoxyinosine
  misc_feature order(1..24,26..30): note = RNA
  misc_feature 25: note = DNA
  modified_base order(1..15,24): mod_base = OTHER; note = 2-Prime-F nucleotides
  modified_base order(16..23,27..30): mod_base = OTHER; note = 2-Prime-OMe nucleotides
  modified_base order(1..2,16..17,26..27,29..30): mod_base = OTHER; note = Phosphoramidate linkage
  modified_base order(2..16,17..26,27..29): mod_base = OTHER; note = Phosphorothioate linkage
cgctccaggttccatgggatgctccnggcg

SEQ ID NO: 42 - DNA, 25 bp, synthetic construct
  source 1..25: mol_type = other DNA
  misc_feature order(1..11,14..25): note = RNA
  misc_feature 12..13: note = DNA
  modified_base order(1..5,7,9,14,18,20,22..25): mod_base = OTHER; note = 2-Prime-OMe nucleotides
  modified_base order(6,8,10,15,17,19,21): mod_base = OTHER; note = 2-Prime-F nucleotides
  modified_base 11: mod_base = OTHER; note = 2-Prime-MOE modified 5-methyl-nucleotide
  modified_base 12: mod_base = OTHER; note = DNA nucleotide carrying a Benner base
  modified_base order(1..2,24..25): mod_base = OTHER; note = Phosphoramidate linkage
  modified_base order(2..13,14..24): mod_base = OTHER; note = Phosphorothioate linkage
  modified_base 13^14: mod_base = OTHER; note = Methylphosphonate linkage
  modified_base 16: mod_base = OTHER; note = 2-Prime-MOE nucleotide
gaaagcaatgcnatcacctcccctg

SEQ ID NO: 43 - DNA, 23 bp, synthetic construct
  source 1..23: mol_type = other DNA
tgctggagctggccttgaagttg

SEQ ID NO: 44 - DNA, 22 bp, synthetic construct
  source 1..22: mol_type = other DNA
ctggtattcatccgcccggtac

SEQ ID NO: 45 - DNA, 16 bp, synthetic construct
  source 1..16: mol_type = other DNA
ttccgaggaggacggc

SEQ ID NO: 46 - DNA, 22 bp, synthetic construct
  source 1..22: mol_type = other DNA
gtctaggagatacacctccacc

SEQ ID NO: 47 - DNA, 21 bp, synthetic construct
  source 1..21: mol_type = other DNA
agtttctccaggagtgggaag

SEQ ID NO: 48 - DNA, 20 bp, synthetic construct
  source 1..20: mol_type = other DNA
agcacctggcaatggcgtag

SEQ ID NO: 49 - DNA, 17 bp, synthetic construct
  source 1..17: mol_type = other DNA
  modified_base 8..10: mod_base = OTHER; note = Locked nucleic acids
ctttgcccagagcatcc

SEQ ID NO: 50 - DNA, 17 bp, synthetic construct
  source 1..17: mol_type = other DNA
  modified_base 8..10: mod_base = OTHER; note = Locked nucleic acids
ctttgcccggagcatcc

SEQ ID NO: 51 - DNA, 18 bp, synthetic construct
  source 1..18: mol_type = other DNA
agcgcatggaggcccaag

SEQ ID NO: 52 - DNA, 20 bp, synthetic construct
  source 1..20: mol_type = other DNA
ctgctggagctgacggaggc

SEQ ID NO: 53 - DNA, 23 bp, synthetic construct
  source 1..23: mol_type = other DNA
tgctggagctggccctgaagttg

SEQ ID NO: 54 - DNA, 17 bp, synthetic construct
  source 1..17: mol_type = other DNA
cttcgcccagagcatcc

SEQ ID NO: 55 - DNA, 16 bp, synthetic construct
  source 1..16: mol_type = other DNA
cttcgcccggagcatc

SEQ ID NO: 56 - DNA, 18 bp, synthetic construct
  source 1..18: mol_type = other DNA
ggcctggccctgaagttg

SEQ ID NO: 57 - DNA, 22 bp, synthetic construct
  source 1..22: mol_type = other DNA
catgctgggataattcgctcca

SEQ ID NO: 58 - DNA, 16 bp, synthetic construct
  source 1..16: mol_type = other DNA
  modified_base 8..10: mod_base = OTHER; note = Locked nucleic acids
cttcgcccggagcatc

SEQ ID NO: 59 - DNA, 17 bp, synthetic construct
  source 1..17: mol_type = other DNA
  modified_base 8..10: mod_base = OTHER; note = Locked nucleic acids
cttcgcccagagcatcc

SEQ ID NO: 60 - DNA, 25 bp, synthetic construct
  source 1..25: mol_type = other DNA
  misc_feature order(1..11,14..25): note = RNA
  misc_feature 12..13: note = DNA
  modified_base order(1..6,14,17..25): mod_base = OTHER; note = 2-Prime-OMe nucleotides
  modified_base order(7,10,16): mod_base = OTHER; note = 2-Prime-MOE nucleotides
  modified_base order(8..9,11): mod_base = OTHER; note = 2-Prime-MOE modified 5-methyl-nucleotides
  modified_base order(1..7,18..25): mod_base = OTHER; note = Phosphorothioate linkage
  modified_base 13^14: mod_base = OTHER; note = Methylphosphonate linkage
  modified_base 15: mod_base = OTHER; note = 2-Prime-F nucleotide
atcactgtcgccatgacaacaccgc

SEQ ID NO: 61 - DNA, 25 bp, synthetic construct
  source 1..25: mol_type = other DNA
  misc_feature order(1..11,14..25): note = RNA
  misc_feature 12..13: note = DNA
  modified_base order(1..6,14,17..25): mod_base = OTHER; note = 2-Prime-OMe nucleotides
  modified_base order(7,10,16): mod_base = OTHER; note = 2-Prime-MOE nucleotides
  modified_base order(8..9,11): mod_base = OTHER; note = 2-Prime-MOE modified 5-methyl-nucleotides
  modified_base order(1..7,15..16,18..25): mod_base = OTHER; note = Phosphorothioate linkage
  modified_base 13^14: mod_base = OTHER; note = Methylphosphonate linkage
atcactgtcgccatgacaacaccgc

SEQ ID NO: 62 - DNA, 25 bp, synthetic construct
  source 1..25: mol_type = other DNA
  misc_feature order(1..11,14..25): note = RNA
  misc_feature 12..13: note = DNA
  modified_base order(1..6,14,17..25): mod_base = OTHER; note = 2-Prime-OMe nucleotides
  modified_base order(7,10,16): mod_base = OTHER; note = 2-Prime-MOE nucleotides
  modified_base order(8..9,11): mod_base = OTHER; note = 2-Prime-MOE modified 5-methyl-nucleotides
  modified_base 15: mod_base = OTHER; note = 2-Prime-F nucleotide
  modified_base order(1..7,12..13,15..17,18..25): mod_base = OTHER; note = Phosphorothioate linkage
  modified_base 13^14: mod_base = OTHER; note = Methylphosphonate linkage
atcactgtcgccatgacaacaccgc

SEQ ID NO: 63 - DNA, 25 bp, synthetic construct
  source 1..25: mol_type = other DNA
  misc_feature order(1..11,14..25): note = RNA
  misc_feature 12..13: note = DNA
  modified_base order(1..6,14,17..25): mod_base = OTHER; note = 2-Prime-OMe nucleotides
  modified_base order(7,10,16): mod_base = OTHER; note = 2-Prime-MOE nucleotides
  modified_base order(8..9,11): mod_base = OTHER; note = 2-Prime-MOE modified 5-methyl-nucleotides
  modified_base 15: mod_base = OTHER; note = 2-Prime-F nucleotide
  modified_base order(1..7,11..13,14..25): mod_base = OTHER; note = Phosphorothioate linkage
  modified_base 13^14: mod_base = OTHER; note = Methylphosphonate linkage
atcactgtcgccatgacaacaccgc

SEQ ID NO: 64 - RNA, 25 nt, synthetic construct
  source 1..25: mol_type = other RNA
  modified_base 13: mod_base = OTHER; note = Deoxyinosine
  modified_base order(1..10,14,17..25): mod_base = OTHER; note = 2-Prime-OMe nucleotides
  modified_base order(11,16): mod_base = OTHER; note = 2-Prime-MOE modified 5-methyl-nucleotides
  modified_base 15: mod_base = OTHER; note = 2-Prime-F nucleotide
  modified_base order(1..11,12..13,15..25): mod_base = OTHER; note = Phosphorothioate linkage
  modified_base 13^14: mod_base = OTHER; note = Methylphosphonate linkage
atgggatgctccnggcgaagacaga

SEQ ID NO: 65 - RNA, 25 nt, synthetic construct
  source 1..25: mol_type = other RNA
  modified_base 13: mod_base = OTHER; note = Deoxyinosine
  modified_base order(1..10,14,17..25): mod_base = OTHER; note = 2-Prime-OMe nucleotides
  modified_base order(11,16): mod_base = OTHER; note = 2-Prime-MOE modified 5-methyl-nucleotides
  modified_base 15: mod_base = OTHER; note = 2-Prime-F nucleotide
  modified_base order(1..11,12..13,15..16,18..25): mod_base = OTHER; note = Phosphorothioate linkage
  modified_base 13^14: mod_base = OTHER; note = Methylphosphonate linkage
atgggatgctccnggcgaagacaga

SEQ ID NO: 66 - RNA, 25 nt, synthetic construct
  source 1..25: mol_type = other RNA
  modified_base 13: mod_base = OTHER; note = Deoxyinosine
  modified_base order(1..10,14,17..25): mod_base = OTHER; note = 2-Prime-OMe nucleotides
  modified_base order(11,16): mod_base = OTHER; note = 2-Prime-MOE modified 5-methyl-nucleotides
  modified_base 15: mod_base = OTHER; note = 2-Prime-F nucleotide
  modified_base order(1..11,18..25): mod_base = OTHER; note = Phosphorothioate linkage
  modified_base 13^14: mod_base = OTHER; note = Methylphosphonate linkage
atgggatgctccnggcgaagacaga

SEQ ID NO: 67 - DNA, 25 bp, synthetic construct
  source 1..25: mol_type = other DNA
  modified_base 13: mod_base = OTHER; note = Deoxyinosine
  modified_base 12: mod_base = OTHER; note = DNA nucleotide carrying a Benner base
  misc_feature order(1..11,13..25): note = RNA
  misc_feature 12: note = DNA
  modified_base order(1..10,14,17..25): mod_base = OTHER; note = 2-Prime-OMe nucleotides
  modified_base order(11,16): mod_base = OTHER; note = 2-Prime-MOE modified 5-methyl-nucleotides
  modified_base 15: mod_base = OTHER; note = 2-Prime-F nucleotide
  modified_base order(1..11,12..13,15..25): mod_base = OTHER; note = Phosphorothioate linkage
  modified_base 13^14: mod_base = OTHER; note = Methylphosphonate linkage
  modified_base 15: mod_base = OTHER; note = 2-Prime-F nucleotide
atgggatgctcnnggcgaagacaga

SEQ ID NO: 68 - DNA, 25 bp, synthetic construct
  source 1..25: mol_type = other DNA
  misc_feature order(1..11,13..25): note = RNA
  misc_feature 12: note = DNA
  modified_base 12: mod_base = OTHER; note = DNA nucleotide carrying a Benner base
  modified_base 13: mod_base = OTHER; note = Deoxyinosine
  modified_base order(1..5,14,22..25): mod_base = OTHER; note = 2-Prime-OMe nucleotides
  modified_base order(6..10,15,17..21): mod_base = OTHER; note = 2-Prime-F nucleotides
  modified_base order(11,16): mod_base = OTHER; note = 2-Prime-MOE modified 5-methyl-nucleotides
  modified_base order(1..11,12..13,15..25): mod_base = OTHER; note = Phosphorothioate linkage
  modified_base 13^14: mod_base = OTHER; note = Methylphosphonate linkage
atgggatgctcnnggcgaagacaga

SEQ ID NO: 69 - DNA, 25 bp, synthetic construct
  source 1..25: mol_type = other DNA
  misc_feature order(1..11,13..25): note = RNA
  misc_feature 12: note = DNA
  modified_base 12: mod_base = OTHER; note = DNA nucleotide carrying a Benner base
  modified_base 13: mod_base = OTHER; note = Deoxyinosine
  modified_base order(1..5,7,9,14,18,20,22..25): mod_base = OTHER; note = 2-Prime-OMe nucleotides
  modified_base order(6,8,10,15,17,19,21): mod_base = OTHER; note = 2-Prime-F nucleotides
  modified_base order(11,16): mod_base = OTHER; note = 2-Prime-MOE modified 5-methyl-nucleotides
  modified_base order(1..11,12..13,15..25): mod_base = OTHER; note = Phosphorothioate linkage
  modified_base 13^14: mod_base = OTHER; note = Methylphosphonate linkage
atgggatgctcnnggcgaagacaga

SEQ ID NO: 70 - DNA, 25 bp, synthetic construct
  source 1..25: mol_type = other DNA
  misc_feature order(1..11,13..25): note = RNA
  misc_feature 12: note = DNA
  modified_base 12: mod_base = OTHER; note = DNA nucleotide carrying a Benner base
  modified_base 13: mod_base = OTHER; note = Deoxyinosine
  modified_base order(1..5,7,9,14,18,20,22..25): mod_base = OTHER; note = 2-Prime-OMe nucleotides
  modified_base order(6,8,10,15,17,19,21): mod_base = OTHER; note = 2-Prime-F nucleotides
  modified_base order(11,16): mod_base = OTHER; note = 2-Prime-MOE modified 5-methyl-nucleotides
  modified_base order(1..2,24..25): mod_base = OTHER; note = Phosphoramidate linkage
  modified_base order(2..11,12..13,15..24): mod_base = OTHER; note = Phosphorothioate linkage
  modified_base 13^14: mod_base = OTHER; note = Methylphosphonate linkage
atgggatgctcnnggcgaagacaga

SEQ ID NO: 71 - RNA, 29 nt, synthetic construct
  source 1..29: mol_type = other RNA
  modified_base 17: mod_base = OTHER; note = Deoxyinosine
  modified_base 16: mod_base = OTHER; note = Cytidine carrying a 2-Prime,2-Prime-difluoro substitution in the ribose moiety
  modified_base order(1..4,15,20): mod_base = OTHER; note = 2-Prime-MOE modified 5-methyl-nucleotides
  modified_base 5: mod_base = OTHER; note = 2-Prime-MOE nucleotide
  modified_base order(6..9,11,13,18,22,24,26..29): mod_base = OTHER; note = 2-Prime-OMe nucleotides
  modified_base order(10,12,14,19,21,23,25): mod_base = OTHER; note = 2-Prime-F nucleotides
  modified_base order(1..2,28..29): mod_base = OTHER; note = Phosphoramidate linkage
  modified_base order(6..15,16..17,19..28): mod_base = OTHER; note = Phosphorothioate linkage
  modified_base 17^18: mod_base = OTHER; note = Methylphosphonate linkage
ttccacgggatgctccnggcaaagacaga

SEQ ID NO: 72 - RNA, 27 nt, synthetic construct
  source 1..27: mol_type = other RNA
  modified_base 15: mod_base = OTHER; note = Deoxyinosine
  modified_base 14: mod_base = OTHER; note = Cytidine carrying a 2-Prime,2-Prime-difluoro substitution in the ribose moiety
  modified_base order(1..2,13,18): mod_base = OTHER; note = 2-Prime-MOE modified 5-methyl-nucleotides
  modified_base 3: mod_base = OTHER; note = 2-Prime-MOE nucleotide
  modified_base order(4..7,9,11,16,20,22,24..27): mod_base = OTHER; note = 2-Prime-OMe nucleotides
  modified_base order(8,10,12,17,19,21,23): mod_base = OTHER; note = 2-Prime-F nucleotides
  modified_base order(1..2,26..27): mod_base = OTHER; note = Phosphoramidate linkage
  modified_base order(4..13,14..15,17..26): mod_base = OTHER; note = Phosphorothioate linkage
  modified_base 15^16: mod_base = OTHER; note = Methylphosphonate linkage
ccacgggatgctccnggcaaagacaga

SEQ ID NO: 73 - RNA, 25 nt, synthetic construct
  source 1..25: mol_type = other RNA
  modified_base 12: mod_base = OTHER; note = Cytidine carrying a 2-Prime,2-Prime-difluoro substitution in the ribose moiety
  modified_base 13: mod_base = OTHER; note = Deoxyinosine
  modified_base 1: mod_base = OTHER; note = 2-Prime-MOE nucleotide
  modified_base order(2..5,7,9,14,18,20,22..25): mod_base = OTHER; note = 2-Prime-OMe nucleotides
  modified_base order(6,8,10,15,17,19,21): mod_base = OTHER; note = 2-Prime-F nucleotides
  modified_base order(11,16): mod_base = OTHER; note = 2-Prime-MOE modified 5-methyl-nucleotides
  modified_base order(1..2,24..25): mod_base = OTHER; note = Phosphoramidate linkage
  modified_base order(2..11,12..13,15..24): mod_base = OTHER; note = Phosphorothioate linkage
  modified_base 13^14: mod_base = OTHER; note = Methylphosphonate linkage
acgggatgctccnggcaaagacaga

SEQ ID NO: 74 - DNA, 27 bp, synthetic construct
  source 1..27: mol_type = other DNA
  misc_feature order(1..13,15..27): note = RNA
  misc_feature 14: note = DNA
  modified_base 14: mod_base = OTHER; note = DNA nucleotide carrying a Benner base
  modified_base 15: mod_base = OTHER; note = Deoxyinosine
  modified_base order(1..2,13,18): mod_base = OTHER; note = 2-Prime-MOE modified 5-methyl-nucleotides
  modified_base 3: mod_base = OTHER; note = 2-Prime-MOE nucleotide
  modified_base order(4..7,9,11,16,20,22,24..27): mod_base = OTHER; note = 2-Prime-OMe nucleotides
  modified_base order(8,10,12,17,19,21,23): mod_base = OTHER; note = 2-Prime-F nucleotides
  modified_base order(1..2,26..27): mod_base = OTHER; note = Phosphoramidate linkage
  modified_base order(4..13,14..15,17..26): mod_base = OTHER; note = Phosphorothioate linkage
  modified_base 15^16: mod_base = OTHER; note = Methylphosphonate linkage
ccatgggatgctcnnggcgaagacaga

SEQ ID NO: 75 - DNA, 27 bp, synthetic construct
  source 1..27: mol_type = other DNA
  misc_feature order(1..13,15..27): note = RNA
  misc_feature 14: note = DNA
  modified_base order(1..2,13,18): mod_base = OTHER; note = 2-Prime-MOE modified 5-methyl-nucleotides
  modified_base 3: mod_base = OTHER; note = 2-Prime-MOE nucleotide
  modified_base order(4..6,16,25..27): mod_base = OTHER; note = 2-Prime-OMe nucleotides
  modified_base order(7..12,17,19..24): mod_base = OTHER; note = 2-Prime-F nucleotides
  modified_base order(4..13,14..15,17..27): mod_base = OTHER; note = Phosphorothioate linkage
  modified_base 15^16: mod_base = OTHER; note = Methylphosphonate linkage
ccatgggatgctcnnggcgaagacaga

SEQ ID NO: 76 - RNA, 27 nt, synthetic construct
  source 1..27: mol_type = other RNA
  modified_base 15: mod_base = OTHER; note = Deoxyinosine
  modified_base 14: mod_base = OTHER; note = Cytidine carrying a 2-Prime,2-Prime-difluoro substitution in the ribose moiety
  modified_base order(1..2,13,18): mod_base = OTHER; note = 2-Prime-MOE modified 5-methyl-nucleotides
  modified_base 3: mod_base = OTHER; note = 2-Prime-MOE nucleotide
  modified_base order(4..7,9,11,16,20,22,24..27): mod_base = OTHER; note = 2-Prime-OMe nucleotides
  modified_base order(8,10,12,17,19,21,23): mod_base = OTHER; note = 2-Prime-F nucleotides
  modified_base order(1..2,26..27): mod_base = OTHER; note = Phosphoramidate linkage
  modified_base order(4..13,14..15,17..26): mod_base = OTHER; note = Phosphorothioate linkage
  modified_base 15^16: mod_base = OTHER; note = Methylphosphonate linkage
ccatgggatgctccnggcgaagacaga

SEQ ID NO: 77 - DNA, 27 bp, synthetic construct
  source 1..27: mol_type = other DNA
  misc_feature order(1..13,15..27): note = RNA
  misc_feature 14: note = DNA
  modified_base order(1..2,13,18): mod_base = OTHER; note = 2-Prime-MOE modified 5-methyl-nucleotides
  modified_base 3: mod_base = OTHER; note = 2-Prime-MOE nucleotide
  modified_base order(4..7,9,11,16,20,22,24..27): mod_base = OTHER; note = 2-Prime-OMe nucleotides
  modified_base order(8,10,12,17,19,21,23): mod_base = OTHER; note = 2-Prime-F nucleotides
  modified_base 14: mod_base = OTHER; note = DNA nucleotide carrying a Benner base
  modified_base 15: mod_base = OTHER; note = Deoxyinosine
  modified_base order(1..2,26..27): mod_base = OTHER; note = Phosphoramidate linkage
  modified_base order(4..15,16..26): mod_base = OTHER; note = Phosphorothioate linkage
  modified_base 15^16: mod_base = OTHER; note = Methylphosphonate linkage
ccatgggatgctcnnggcgaagacaga

SEQ ID NO: 78 - DNA, 30 bp, synthetic construct
  source 1..30: mol_type = other DNA
  misc_feature order(1..24,26..30): note = RNA
  misc_feature 25: note = DNA
  modified_base order(1..15,24): mod_base = OTHER; note = 2-Prime-F nucleotides
  modified_base order(16..23,27..30): mod_base = OTHER; note = 2-Prime-OMe nucleotides
  modified_base 25: mod_base = OTHER; note = DNA nucleotide carrying a Benner base
  modified_base 26: mod_base = OTHER; note = Deoxyinosine
  modified_base order(1..2,16..17,26..27,29..30): mod_base = OTHER; note = Phosphoramidate linkage
  modified_base order(2..16,17..26,27..29): mod_base = OTHER; note = Phosphorothioate linkage
cgctccaggttccatgggatgctcnnggcg

SEQ ID NO: 79 - RNA, 25 nt, synthetic construct
  source 1..25: mol_type = other RNA
caggggaggtgatagcattgctttc

(19) United States
(12) Patent Application Publication
(10) Pub. No.: US 2025/0154504 A1
(43) Pub. Date: May 15, 2025

(54) GUIDE OLIGONUCLEOTIDES FOR NUCLEIC ACID EDITING IN THE TREATMENT OF HYPERCHOLESTEROLEMIA

(71) Applicant: ProQR Therapeutics II B.V., Leiden (NL)

(72) Inventors: Gerardus Johannes PLATENBURG, Leiden (NL); Bart KLEIN, Leiden (NL); Lenka VAN SINT FIET; Maarten HOLKERS, Leiden (NL)

(73) Assignee: ProQR Therapeutics II B.V., Leiden (NL)

(21) Appl. No.: 18/838,201

(22) PCT Filed: Feb. 13, 2023
(86) PCT No.: PCT/EP2023/053503

§ 371 Date: Aug. 13, 2024

(30) Foreign Application Priority Data
Feb. 14, 2022 (GB) 2201902.0
Oct. 28, 2022 (GB) 2215990.9
Nov. 14, 2022 (GB) 2216943.7

(51) Int. Cl. C12N 15/11; A61K 31/7105; A61P 7/00; C12N 15/86

(52) U.S. Cl. CPC C12N 15/111; A61K 31/7105; A61P 7/00; C12N 15/86; C12N 2310/315; C12N 2310/351; C12N 2750/14143

(57) ABSTRACT

The invention relates to guide oligonucleotides that can bring about specific changes to a target RNA or DNA molecule in a eukaryotic cell, preferably liver cells, for use in the treatment of hypercholesterolemia, cardiovascular disease, liver injury and/or alcohol-induced steatohepatitis in human subjects. More specifically, the invention relates to guide oligonucleotides and the use thereof in the editing of nucleic acids encoding an auto-cleavage site within the PCSK9 proprotein, thereby inhibiting, or inactivating the PCSK9 protein (or the matured PCSK9 complex) in its ability to cause LDL receptor protein degradation.

### CROSS-REFERENCE TO RELATED APPLICATIONS

This application is a 35 U.S.C. § 371 National Phase Application of PCT/EP2023/053503, filed Feb. 13, 2023, which claims the benefit of British Application No. GB 2201902.0, filed Feb. 14, 2022, British Application No. GB 2215990.9, filed Oct. 28, 2022 and British Application No. 2216943.7, filed Nov. 14, 2022.

### REFERENCE TO SEQUENCE LISTING SUBMITTED ELECTRONICALLY

The content of the electronically submitted sequence listing in XML format (Name 4430_0220003_SeqListing_ST26.xml; Size: 228,792 bytes; and Date of Creation: Aug. 15, 2024) filed with the application is incorporated herein by reference in its entirety.

### FIELD OF THE INVENTION

The present invention is in the field of medicine and biotechnology. More in particular, the invention is in the field of modifying nucleic acids, such as DNA or RNA, to alter proteins involved in disease, like changing the fate of proteins involved in pathways leading to disease. It relates to guide oligonucleotides that are applicable in influencing the function and/or activity of wild type or gain-of-function mutants of Proprotein Convertase Subtilisin/Kexin type 9 (PCSK9) in patients suffering from cardiovascular disease (such as (familial) hypercholesterolemia), liver injury, and/or alcoholic liver disease.

### BACKGROUND OF THE INVENTION

The PCSK9 enzyme is central in the regulation of the LDL receptor, a receptor involved in the uptake of Low-Density Lipoprotein Cholesterol (LDL-C; the “bad” cholesterol) from the blood. PCSK9 is predominantly synthesized in the liver in hepatocytes. In patients with hypercholesterolemia, familial or otherwise, wherein high levels of plasma circulating LDL-C is present, the LDL receptor is downregulated and/or degraded upon interaction with the PCSK9 protein, a process that requires processing of the PCSK9 proprotein by autocleavage at the P1 site (Benjannet S et al. J Biol Chem. 2012 287(40):33745-33755). Strategies to treat hypercholesterolemia (to lower plasma LDL-C levels) and/or the cardiovascular diseases caused thereby or associated with it, based on targeting the PCSK9 protein have been employed, for instance using small molecule inhibitors or monoclonal antibodies. The inhibitor may prevent the interaction between PCSK9 and the LDL receptor thereby rescuing the receptor function and/or its recycling. Small molecule inhibitors that target specific domains or amino acid residues of the PCSK9 protein, especially the one interacting with the LDL receptor and causing it to be downregulated, are known. Drawback of small molecule inhibitors are lack of specificity and potential side effects. Alternative strategies exist that target a nucleic acid coding for PCSK9. Strategies to target RNA coding for PCSK9 based on RNaseH1 mediated knock-down using oligonucleotides called ‘gapmers’ or RISC-based downregulation using small interfering (or short interference) RNA molecules (siRNAs) have been explored for therapy for patients with hypercholesteremia. Moreover, initiatives have been announced to apply DNA base-editing to target the PCSK9 gene.

Interfering with proteins on a nucleic acid level to prevent or change the course of a disease is not a new concept. Since the 1980s experimenters have embarked on restoring protein production in cells that have lost the capacity to make that protein because of mutations in the gene coding for that protein. This approach, typically referred to as gene therapy, requires a healthy copy of the gene to be delivered to the defective cells. The delivery is not very efficient, can lead to unwanted side-effects such as mutations of resident genes upon integration into the genome of the patient and requires delivery vehicles that come with certain drawbacks, such as limited ‘packaging capacity’ and/or the triggering of immune reactions and/or occurrence of pre-existing immunity against vector components in the host (for example a patient in need of treatment). This limits the applicability of gene therapy to situations where the corrected gene is fairly limited in size, ‘one-and-done’ treatment situations, and situations where the disease involves a loss of function due to the gene defect. Although progress has been made in the field of gene therapy, it is not a strategy for all types of genetic disease and not all the drawbacks have been solved.

More recently, gene editing technologies, such as TALEN and CRISPR/Cas-based systems have been developed with great promise to treat many genetic diseases. Instead of adding a gene to the cells' genome, as with classical gene therapy described above, these systems are designed to make changes in the genome of the patient by delivering machinery that can make changes to the target gene in situ, i.e., directly to the DNA in the patients' cells. This form of genome editing can be extremely powerful to treat genetic diseases if it is possible to take out cells out of the patient's body, edit them and subsequently re-introducing the corrected cells into the patient after selection for the correct edits. These so-called ex vivo treatments are limited to cells that can be isolated relatively easily from the patient, such as blood cells, but not for cells that cannot be taken out of the body and/or cannot be re-inserted in the right tissue after correction. Despite early successes, this form of gene editing is still in its infancy and comes with risks associated with DNA breaks and imprecise repair, off-target edits, and complexities and risks associated with the delivery of the editing machinery and the guides to direct the edits (for example immunogenicity against foreign proteinaceous components, such as enzyme parts or proteinaceous parts of the delivery vehicle).

A more recent development is the advent of base editing. Base editing is different from classical gene editing, in that the nucleic acid strands of the target gene are left intact. In other words, contrary to classical gene editing, where the strands must be broken to trigger the cell's natural DNA repair systems, base editing does not require breakage of the phosphodiester backbone linking the nucleotides in DNA or RNA strands. Instead, enzymes that work on the nucleobase itself enzymatically convert the amino group in the purine (adenosine) or pyrimidine (cytidine) ring, converting these amine groups into oxygen (keto-group), thereby changing an adenosine into an inosine and a cytidine into a thymidine (DNA) or uridine (RNA). These conversions change the base pairing properties of the nucleobases affecting structure of RNA function, micro-RNA target RNA interactions, or transfer-RNA recognition, to name a few examples. Consequently, inosines, generated by ADAR edits in a double strand RNA (or DNA), are interpreted as guanosine for base pairing and translation purposes. Effectively, ADAR based editors bring about an A to G change in the case of adenosine base editors and a C to U change in the case of cytidine editors. In mRNA, or its precursors, edits can directly change the amino acid depending on the position of the target base in the codon. Any edits made on the DNA level are faithfully copied into RNA by the process of transcription.

For the sake of efficiency, the benefits and applications of targeted base editing will be explained in more detail in the current disclosure by referring to RNA, but it will be clear to those of skill in the art that the principles can be applied to DNA edits in the same way with adaptations that relate to the editors (the deaminase enzymes) or the guide oligonucleotides that are required to make the edits target-specific.

Recently, it has been shown that ADAR enzymes can be manipulated (for example by truncation, fusion to effector domains, or mutation of the deaminase domain to alter its properties) or recruited ‘as is’ from within the target cells where they normally reside, to act on any target DNA or RNA selected by the experimenter or clinician.

What is required in addition to the editor (the deaminase enzyme) is a guide to recruit the editor to the target of choice. Typically, herein, a guide is referred to as a ‘guide oligonucleotide’ that comprises a string of nucleotides that is substantially complementary to the target sequence containing the base targeted to be edited (A or C) in the sense of Watson-Crick base pairing. In the case of RNA editing by ADARs, what is required is a stretch of RNA (or synthetic derivatives thereof) of sufficient length to form a double-stranded RNA complex by Watson-Crick base pairing, to recruit the editor (which may be a natural ADAR enzyme or a modified form thereof, or a fusion between ADAR and unrelated protein domains) as-long-as it comprises a functional deaminase domain). Details of various editor variants in use for RNA and DNA base editing, as well as different strategies and designs of guide oligonucleotides are readily available in the prior art (illustrative examples of references are discussed below).

As will be readily apparent to those versed in the art of molecular biology and genetics, the advent of techniques of nucleic acid editing brings a wealth of opportunities to tackle diseases that are otherwise not or difficult to treat. Among the monogenic diseases, there are thousands of instances where the mutation of a single base is responsible for the disease and there are often well described causal relationships between the mutation and the disease phenotype. Treatment of the disease is based on the correction of the mutated base to restore proper gene function and further downstream, protein function. Beyond these forms of monogenic diseases that are based on the correction of the mutation on the nucleic acid level, there lies a universe of diseases that do not have a one-on-one relationship with a mutation in some gene. Some diseases are multigenic in nature, involving a host of different genes coding for proteins that have complex interactions with each other, the nature of which is not always entirely understood. In such instances, there is not a single gene, let alone a single base to be corrected to restore healthy functioning. Sometimes, although the genetic nature of the disease may not always be understood, the relationship between two or more proteins interacting with each other is sufficiently understood to allow an intervention that improves the disease state. One example of a situation described above is one wherein a change in the function (or localization) of one protein, positively reflects on the function of another one, is PCSK9.

### SUMMARY OF THE INVENTION

The present invention relates to a guide oligonucleotide for use in the treatment, delay, or prevention of hypercholesterolemia, cardiovascular disease, liver injury and/or alcohol-induced steatohepatitis, wherein the oligonucleotide is capable of inducing editing of a nucleic acid encoding a human PCSK9 proprotein, and wherein the nucleic acid editing decreases or prevents the ability of the PCSK9 proprotein from being processed by auto-cleavage of a proteolytic cleavage site. Preferably, the proteolytic cleavage site is the P1 cleavage site, and the nucleic acid that is edited is RNA or DNA, which means that the editing can take place in the genome (DNA) or on a level of pre-mRNA or mRNA. In the present disclosure, RNA editing is described in detail and the invention is exemplified by RNA editing procedures, but the invention relates to at least both levels of nucleic acid editing.

In one embodiment of the invention, the nucleic acid to be edited is RNA and the guide oligonucleotide can form a double stranded complex with a human PCSK9 pre-mRNA or mRNA, or a part thereof, wherein the guide oligonucleotide when complexed with the PCSK9 pre-mRNA or mRNA is able to engage an enzyme comprising adenosine deaminase activity, preferably an endogenous ADAR enzyme, thereby allowing the deamination of a target adenosine in the human PCSK9 pre-mRNA or mRNA. Preferably, the guide oligonucleotide comprises an orphan base that is a cytidine or a cytidine analog. A preferred cytidine analog is a Benner's base (dZ), as disclosed in WO 2020/252376. The orphan base is the base opposite the target adenosine in the target nucleic acid to which the guide oligonucleotide is substantially complementary. In a preferred embodiment, the target adenosine is the second nucleotide of the codon coding for the glutamine residue at position 152 in the PCSK9 proprotein. This position is herein also referred to as Gln152. The Gln152 residue is part of the P1 cleavage site of the PCSK9 proprotein. In another preferred embodiment, the guide oligonucleotide comprises or consists of 20 to 50 nucleotides, more preferably 23 to 27 nucleotides, and wherein the guide oligonucleotide is substantially complementary to a region within the human PCSK9 pre-mRNA or mRNA that comprises the codon coding for the glutamine residue at position 152 in the PCSK9 proprotein. This means that the complementary sequence of the guide oligonucleotide overlaps with the position of Gln152 and can comprise additional (complementary and non-complementary) nucleotides towards its 5′ and 3′ terminus. In another preferred embodiment, the guide oligonucleotide is conjugated (bound) to a GalNAc moiety. In one embodiment of the present invention, the guide oligonucleotide comprises one or more nucleotides that comprise a modification of a nucleobase, a sugar and/or an inter-nucleosidic linkage.

The present invention also relates to viral vector expressing a guide oligonucleotide as characterized herein, wherein the viral vector is for use in the treatment, delay, or prevention of hypercholesterolemia, cardiovascular disease, liver injury and/or alcohol-induced steatohepatitis, wherein the oligonucleotide is capable of editing a nucleic acid encoding a human PCSK9 proprotein, and wherein the nucleic acid editing decreases or prevents the ability of the PCSK9 proprotein from being processed by auto-cleavage of a proteolytic cleavage site. The present invention also relates to a pharmaceutical composition comprising a guide oligonucleotide as characterized herein or a viral vector as characterized herein, and a pharmaceutically acceptable carrier.

In yet another embodiment, the invention relates to a method for treating a human subject suffering from hypercholesterolemia by decreasing or inhibiting an auto-cleaving ability of the PCSK9 proprotein in a liver cell of the subject, the method comprising administering to the subject a guide oligonucleotide as characterized herein, a viral vector as characterized herein, or a pharmaceutical composition as characterized herein, thereby editing a nucleic acid encoding the PCSK9 proprotein. Preferably, the auto-cleavage site is the P1 cleavage site of PCSK9 and the nucleic acid to be edited is RNA or DNA. In a preferred embodiment, the nucleic acid is RNA and the guide oligonucleotide can form a double stranded complex with a human PCSK9 pre-mRNA, or mRNA, or a part thereof, wherein the guide oligonucleotide when complexed with the PCSK9 pre-mRNA or mRNA is able to engage an enzyme comprising deaminase activity, thereby allowing the deamination of a target adenosine in the pre-mRNA or mRNA at the P1 cleavage site. Preferably, the adenosine in the codon encoding glutamine at position 152 of the PCSK9 proprotein is deaminated to become an inosine.

### BRIEF DESCRIPTION OF THE DRAWINGS

FIG. 1 shows in the upper panel part of the RNA sequence of the human Amyloid Precursor Protein (hAPP) target sequence from 5′ to 3′ and directly below it the complementary sequence (from 3′ to 5′) of the guide oligonucleotide. The adenosine targeted for editing is indicated in bold and the opposing cytosine (orphan base) in the guide oligonucleotide sequence is underlined. In the lower panel the modifications in the four versions of the guide oligonucleotide are provided: hAPPEx17_33, hAPPEx17_35, hAPPEx17_36, and hAPPEx17_37. An asterisk indicates a phosphorothioate (PS) linkage between two nucleosides. Lower case nucleotides are modified with 2′-OMe. Upper-case italic nucleotides are modified with 2′-MOE. dC and dA nucleotides are DNA. Upper-case underlined nucleotides are modified with 2′-F. The symbol “^{A}” refers to a methylphosphonate (MP) linkage.

FIG. 2 shows the editing percentage of the hAPP target RNA over time in an in vitro biochemical assay using the four guide oligonucleotides of FIG. 1.

FIG. 3 shows the editing percentage after transfection of the four guide oligonucleotides of FIG. 1 in human RPE cells, targeting endogenous hAPP. All oligonucleotides were able to recruit endogenous ADAR for editing. Negative controls were a non-transfected sample (NT) and a mock transfected sample.

FIG. 4 shows in the upper panel the sequence of the Homo sapiens and Macaca mulatta (pre-) mRNAs surrounding the codon (CAG in bold) encoding the glutamine at position 152 in the (human) PCSK9 proprotein from 5′ to 3′. The area for which a variety of editing oligonucleotides was designed is underlined in both sequences. Differences between human and monkey sequences are shaded. In the lower panel the sequence of the guide oligonucleotides are given (5′ to 3′), with PCSK9-1 having the same sequence (now 5′ to 3′) as in the upper panel. Oligonucleotides with the name PCSK9-1, -2, -3, -4, -5, -6, -7, -8, -9, -10, -11, -12, -13, -31, -32, -33, -34, -35, -36, -37, -38, -39, -41, -44, -45, -46, -48, 49, -and -50 are complementary to the monkey sequence. Oligonucleotides with the name PCSK9-17, -18, -19, -23, -24, and -25 are complementary to the human sequence. The chemical modifications in each of the AONs are provided as follows: an asterisk indicates a PS linkage between two nucleosides; lower case nucleotides are modified with 2′-OMe; upper-case italic nucleotides are modified with 2′-MOE, wherein U represents a 2′-MOE modified T (=5-methyl-U) and C represents a 2′-MOE modified 5-methyl-C; dC and dA are DNA; upper-case underlined nucleotides are modified with 2′-F; I is deoxyinosine (opposite a C in the target sequence). Z means a DNA nucleotide carrying a Benner's base (a cytidine analog as disclosed in WO 2020/252376). C_{f2} is cytidine carrying a 2′,2′-difluoro substitution in the ribose moiety. The linkage 3′ from I is a methylphosphonate (MP) linkage given by the “A” symbol; the “!” symbol indicates a phosphoramidate (PNdmi) linkage.

FIGS. 5A, 5B and 5C show the editing percentage of the human PCSK9 target RNA over time in an in vitro biochemical assay using the four guide oligonucleotides of FIG. 4, divided over three different panels. FIG. 5A: PCSK-1, PCSK-2, PCSK-3, PCSK-4 and PCSK-5. FIG. 5B: PCSK-6, PCSK-7, PCSK-8 and PCSK-9. FIG. 5C: PCSK-10, PCSK-11, PCSK-12, and PCSK-13.

FIG. 6 shows in the upper panel part of the mouse Alpha-L-iduronidase RNA sequence (mldua) from 5′ to 3′ and directly below it the complementary sequence of the area of the proposed antisense oligonucleotides from 3′ to 5′. The adenosine targeted for editing is indicated in bold and opposing orphan base cytosine, which forms the mismatch with the targeted A is underlined. In the lower panel the sequences are provided for the six guide oligonucleotides mlDUA-1IVT, mlDUA-5IVT, mlDUA-6IVT, GalNac-mlDUA-1IVT, GalNac-mlDUA-5IVT and GalNac-mlDUA-6IVT from 5′ to 3′. An asterisk indicates a PS linkage; lower case nucleotides are modified with 2′-OMe; upper-case italic nucleotides are modified with 2′-MOE; dC and dA are DNA; upper-case underlined nucleotides are modified with 2′-F; the symbol “\hat{ }” refers to a MP linkage. GalNac-mlDUA-1IVT, GalNac-mlDUA-5IVT and GalNac-mlDUA-6IVT all carry the conjugated GalNac moiety at the 5′ terminus.

FIG. 7—shows the editing percentage of the mldua target RNA over time in an in vitro biochemical assay, achieved by using the six guide oligonucleotides of FIG. 6. All guide oligonucleotides were able to yield very efficient RNA editing.

FIG. 8 shows the percentage editing in endogenous human PCSK9 target RNA after transfection of oligonucleotides into human HeLa cells.

FIG. 9 shows the percentage editing in endogenous human PCSK9 target RNA after gymnotic uptake of oligonucleotides into human primary hepatocytes.

FIG. 10A shows the target sequence of the human Actin B transcript with the target adenosine in bold and underlined. Below the target sequence are given two oligonucleotides for targeting the human Actin B transcript: RM4266 and RM4489 are identical in sequence and chemical modifications, except that RM4489 is bound to GalNAc at the 5′ terminus. Chemical modifications are as described in the legend of FIG. 4. Z is the orphan nucleotide opposite the target adenosine.

FIG. 10B shows the percentage editing in endogenous Actin B target RNA after gymnotic uptake of an oligonucleotide that is attached to a GalNAc moiety (RM4489) on the 5′ terminus and an identical oligonucleotide without the GalNAc moiety (RM4266) in human HepG2 cells, in a dose-dependent manner.

FIG. 11A shows the percentage editing in endogenous human PCSK9 target RNA after transfection of the indicated oligonucleotides (respectively PCSK9-17, -18, -19, -23, -24, and -25) into human HeLa cells.

FIG. 11B shows the amount of beta-tubulin protein in the samples 48 hrs after transfection of the EONs, used for normalization.

FIG. 11C shows the amount of total PCSK9 protein as determined on a western blot using an antibody directed against human PCSK9 protein, taking the mock transfected cells as the standard (1.0).

FIG. 11D shows the ratio between cleaved PCSK9 (grey; PCSK9) and un-cleaved PCSK9 protein (black; Pro-PCSK9) together adding up to the total amount of PCSK9 protein shown in (C), after transfection with the indicated oligonucleotides.

FIG. 12 shows the percentage editing in endogenous monkey PCSK9 target RNA after gymnotic uptake of oligonucleotides into non-human primate (NHP) primary hepatocytes.

FIG. 13 shows the percentage editing in endogenous mouse PCSK9 target RNA after gymnotic uptake of guide oligonucleotides with or without the co-administration of a triterpene glycoside (AG1856 is used as an example) into primary mouse hepatocytes.

### DETAILED DESCRIPTION

While small molecules and monoclonal antibodies have been developed to inhibit Proprotein Convertase Subtilisin/Kexin type 9 (PCSK9) protein to treat (familial) hypercholesterolemia, and (antisense) oligonucleotides as well as small and short interfering RNA (siRNA) molecules were developed for inhibiting the activity of PCSK9, the inventors of the present invention have sought for alternative means to inhibit the activity of PCSK9 and therethrough to allow the LDL receptor to be recycled in the cell and bind (and destroy) low-density lipoprotein (LDL) cholesterol from circulation. The inventors envisioned that targeting the site in the protein itself that is responsible for self-cleavage of PCSK9 (the P1 site) would diminish its activity. The means to target the cleavage site, as outlined herein, is preferably by RNA editing, to site-specifically deaminate—in the (pre-) mRNA of the gene—the adenosine in the codon CAG, coding for glutamine (Gln) at position 152, which represents the cleavage site. Converting the CAG codon to CIG (read as CGG by the translation machinery of the cell) would render it to a codon coding for arginine (Arg). The inventors envisioned that not only wild type (pre-) mRNA of PCSK9 could be targeted, but also gain-of-function mutants that are over-actively causing the LDL receptor proteins to be degraded, and thereby to not only treat normal hypercholesterolemia but also hereditary (familial) hypercholesterolemia (FH). Without wishing to be bound by theory, it has been postulated that the loss of autocleavage at the P1 site in the PCSK9 zymogen, prevents PCSK9 from entering the secretory pathway. Under normal circumstances, the PCSK9 proprotein is cleaved at P1, causing the cleaved pro-peptide to form a complex with the mature PCSK9 enzyme. It is the complex that is believed to enter the secretory pathway, enabling it to interact with the LDL receptor, possibly already in the endoplasmic reticulum, causing the LDL receptor to end up in the endosome and/or lysosome of the cell, where the acidification of these compartments induces LDL receptor degradation (Ogura M. J Cardiol. 2018 71(1):1-7). Alternatively, or additionally, a second pathway leading to LDL receptor degradation has been postulated that involves the interaction of the pro-peptide/PCSK9 complex with LDL receptor at the cell surface, after secretion of the former from the cell. Preventing autocleavage of the PCSK9 proprotein prevents both LDL receptor degradation pathways. A further effect of the prevention of autocleavage is that the PCSK9 proprotein is believed to bind to mature PCSK9 that escaped the autocleavage preventing edit, thereby enhancing the LDL receptor protecting effect (Benjannet S et al. 2012). Importantly, it was shown that a known PCSK9 mutation at the 152 position, referred to as Q152H wherein the glutamine is mutated to an histidine, also results in an uncleavable form of proPCSK9 that is retained in the endoplasmic reticulum (ER) of hepatocytes, where it is expected to contribute to ER storage disease (ERSD), a heritable condition known to cause systemic stress and liver injury. It was reported that members of several French-Canadian families known to carry the Q152H mutation exhibited marked hypocholesterolemia and normal liver function despite their lifelong state of ER PCSK9 retention (Lebaeu P F et al. J Clin Invest. 2021. 131(2):e128650). Introducing a hepatic overexpression of the Q152H mutant in mice greatly increased the stability of ER stress response chaperones in hepatocytes and protected against ER stress and liver injury. This supports the idea that introducing a mutation in the PCSK9 transcript in vivo, at the transcript position related to position 152 in the protein, would results in a uncleavable PCSK9 protein. According to the teaching of the present invention this will not only prevent LDL receptor degradation but may also prevent liver injury. Given the marked LDL-C lowering efficacy of PCSK9 inhibition observed with anti-PCSK9 antibody therapeutics, and the prevalence of the underlying disease against which this strategy is targeted, the need for additional PCSK9-inhibitory treatment modalities is well justified (Seidah H G et al. Cardiovasc Res. 2019. 115(3):510-518; Latimer J et al. J Thromb Thrombolysis. 2016. 42(3):405-419; Sabatine M S et al. Postgrad Med. 2016. 128:31-39). On top of the effects observed regarding lowering plasma levels of LDL-C and the protection against liver injury, it was furthermore shown that inhibiting PCSK9 by using the monoclonal antibody Alirocumab attenuated alcohol-induced steatohepatitis (Lee J S et al. Sci Rep. 2019. 9(1):17161).

The present invention relates to the generation of a clinically relevant loss of function PCSK9 variant (Q152R) by administering antisense oligonucleotides to target the wildtype PCSK9 transcript. The present invention provides a new and alternative way of inhibiting PCSK9 activity in hepatocytes and basically provides a new and alternative way of treating the liver-related disorders listed above, such as hypercholesterolemia, cardiovascular disease related to hypercholesterolemia, liver injury, and alcoholic liver disease (such as alcohol-induced steatohepatitis).

The inventors contemplated using a technology generally referred to as ‘RNA editing’. Although, the proposed changes to PCSK9 may be made on the DNA level, and this is considered to form part of the present invention, it is considered an advantage of editing RNA in that the changes are not permanent, can be tuned by adapting the dose or dosing regimen and can even be withdrawn altogether depending on the situation of the hypercholesterolemia patient and the patient's need of treatment.

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms such as Caenorhabditis elegans, to humans. Examples of RNA editing are adenosine (A) to inosine (I) and cytidine (C) to uridine (U) conversions through enzymes called adenosine deaminase and cytidine deaminase, respectively. The most extensively studied RNA editing system is the adenosine deaminase enzyme, which is a multi-domain protein, comprising a recognition domain and a catalytic domain. The recognition domain recognizes a specific double-stranded RNA (dsRNA) sequence and/or conformation, whereas the catalytic domain converts an adenosine into an inosine in a nearby, predefined position in the target RNA, by deamination of the nucleobase. Inosine is read as guanosine by the translational machinery of the cell, meaning that, if an edited adenosine is in a coding region of an mRNA or pre-mRNA, it can recode the protein sequence. RNA editing by adenosine deamination is therefore a perfect way of repairing G>A mutations, but also other mutations wherein conversion from A to G would allow the generation of a functional protein. In the present invention it is a perfect way to deactivate a wild-type protein because the conversion from an A to G in the codon for the wild-type autocleavage site in PCSK9 (Gln152) would render it unable to cleave itself. The adenosine deaminases are part of a family of enzymes referred to as Adenosine Deaminases acting on RNA (ADAR), including human deaminases hADAR1, hADAR2 and hADAR3.

The use of oligonucleotides to edit target RNA applying adenosine deaminase is known in the art. Montiel-Gonzalez et al. (Proc Natl Acad Sci USA 2013, 110(45):18285-18290) described the editing of a target RNA using a genetically engineered fusion protein, comprising an adenosine deaminase domain of the hADAR2 protein, fused to a bacteriophage lambda N protein, which recognises the boxB RNA hairpin sequence. A disadvantage of this method in a therapeutic setting is the need for the fusion protein. It requires cells to be either transduced with the fusion protein, which is a major hurdle, or that target cells are transfected with a nucleic acid construct encoding the engineered adenosine deaminase fusion protein for expression. Vogel et al. (2014. Angewandte Chemie Int Ed 53:267-271) disclosed editing of RNA coding for eCFP and Factor V Leiden, using a benzylguanosine substituted guide RNA and a genetically engineered fusion protein, comprising the adenosine deaminase domains of ADAR1 or 2 (lacking the dsRNA binding domains) genetically fused to a SNAP-tag domain (an engineered 06-alkylguanosine-DNA-alkyl transferase). This system suffers from similar drawbacks as the engineered ADARs described by Montiel-Gonzalez et al. (2013). Woolf et al. (1995. Proc Natl Acad Sci USA 92:8298-8302) disclosed a simpler approach, using relatively long single-stranded antisense RNA oligonucleotides (25-52 nucleotides in length) wherein the longer oligonucleotides (34-mer and 52mer) could promote editing of the target RNA by endogenous ADAR because of the double-stranded nature of the target RNA and the hybridizing oligonucleotide, but only appeared to function in cell extracts or in amphibian (Xenopus) oocytes by microinjection, and suffered from severe lack of specificity: nearly all adenosines in the target RNA strand that was complementary to the antisense oligonucleotide were edited. Woolf et al. (1995) did not achieve deamination of a specific target adenosine in the target RNA sequence, because nearly all adenosines opposite an unmodified nucleotide in the antisense oligonucleotide were edited through a process sometimes referred to as ‘promiscuous editing’. WO2016/097212 discloses RNA editing oligonucleotides, characterized by a sequence that is complementary to a target RNA sequence (‘targeting portion’) and by the presence of a stem-loop structure (‘recruitment portion’). Similar stem-loop structure-comprising systems for RNA editing have been described in WO2017/050306, WO2020/001793, WO2017/010556, WO2020/246560, and WO2022/078995. WO2017/220751 and WO2018/041973 describe a next generation type of AONs that do not comprise such a stem-loop structure but that are (almost fully) complementary to the targeted area. In one embodiment, one or more mismatching nucleotides, wobbles, or bulges exist between the oligonucleotide and the target sequence. A sole mismatch may be at the site of the nucleoside opposite the target adenosine, but in other embodiments AONs (or RNA editing oligonucleotides, abbreviated to ‘EONs’) were described with multiple bulges and/or wobbles when attached to the target sequence area. It appeared possible to achieve in vitro, ex vivo and in vivo RNA editing with EONs lacking a stem-loop structure and with endogenous ADAR enzymes when the sequence of the EON was carefully selected such that it could attract/recruit ADAR. The ‘orphan nucleoside’, which is defined as the nucleoside in the EON that is positioned directly opposite the target adenosine in the target RNA molecule, did not carry a 2′-OMe modification. The orphan nucleoside can be a deoxyribonucleoside (DNA), wherein the remainder of the EON could still carry 2′-O-alkyl modifications at the sugar entity (such as 2′-OMe), or the nucleotides directly surrounding the orphan nucleoside contained chemical modifications (such as DNA in comparison to RNA) that further improved the RNA editing efficiency and/or increased the resistance against nucleases. Such effects could even be further improved by using sense oligonucleotides (SONs) that ‘protected’ the EONs against breakdown (described in WO2018/134301). The use of chemical modifications and particular structures in oligonucleotides that could be used in ADAR-mediated editing of specific adenosines in a target RNA have been the subject of numerous publications in the field, such as WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345. The use of specific sugar moieties has been disclosed in for instance WO2020/154342, WO2020/154343, WO2020/154344, WO2022/103839, and WO2022/103852, whereas the use of stereo-defined linker moieties (in general for oligonucleotides that for instance can be used for exon skipping, in gapmers, in siRNA, or specifically for RNA-editing oligonucleotides, related to a wide variety of target sequences) has been described in WO2011/005761, WO2014/010250, WO2014/012081, WO2015/107425, WO2017/015575 (HTT), WO2017/062862, WO2017/160741, WO2017/192664, WO2017/192679 (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2019/217784 (DM1), WO2019/219581, WO2020/118246 (DM1), WO2020/160336 (HTT), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, and WO2022/099159. Next to these disclosures, an extensive number of publications relate to the targeting of specific RNA target molecules, or specific adenosines within such RNA target molecules, be it to repair a mutation that resulted in a premature stop codon, or other mutation causing disease. Examples of such disclosures in which adenosines are targeted within specified target RNA molecules are WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); and WO2021/243023 (SERPINA1). WO2019/005884 discloses a system for deamination of target adenosines in which a targeting system comprises a targeting domain (a CRISPR system comprising a CRISPR effector protein (such as Cas13) and a guide molecule that is generally an oligonucleotide with a sequence that is complementary to the target sequence) linked to an adenosine deaminase, or a catalytic domain thereof.

In one embodiment, the invention relates to a method to change a protein associated with human cardiovascular disease, such as PCSK9, to alter its processing, affecting its regulation (such as activation) and/or or localization, by editing a nucleic acid coding for that protein. The processing, regulation and/or localization preferably comprises proteolytic cleavage of the proprotein, more preferably autocleavage of a proprotein. An especially preferred target is a nucleic acid encoding the human protein PCSK9, and the preferred processing is the autocleavage of the PCSK9 proprotein to yield the pro-peptide and the mature PCSK9, which can form a complex to become active. More in particular, it is preferred that the editing event prevents or reduces the autocleavage, which contributes to prevention or reduction of occurrence of the active complex in its active compartment. In case the protein is PCSK9, prevention of autocleavage leads to reduced active protein in its active compartment, with the effect that the degradation of the LDL receptor is inhibited or reduced. Preferably, the protein is human PCSK9 and preferably the editing process changes the glutamine at position 152 (Q152, or Gln152, also known as the P1 cleavage site) of the proprotein. A change may be a change of glutamine to an alternative amino acid at the same position or the removal of the target amino acid, for instance through exon skipping. More preferably, the invention relates to an edit that comprises a change of an adenosine in the glutamine codon (Gln152: CAG) that changes it into an arginine codon (Arg152: CGG), thereby effectuating a Q152R substitution in the PCSK9 proprotein.

In another embodiment, a method is provided to change a protein, such as PCSK9 associated with human disease, such as hypercholesterolemia, to alter the protein's processing, affecting its regulation (activation or deactivation) and/or localization, wherein the editing comprises the deamination of a base, such as an adenosine or cytidine. According to a more preferred embodiment, the editing comprises a change of an adenosine into inosine, preferably by an adenosine base editor. According to a more preferred embodiment, the change is brought about by an edit of an adenosine in RNA coding for the protein, by an adenosine base editor that acts on double stranded RNA. In accordance therewith, the adenosine base editor may be a natural ADAR, a truncated ADAR, a fusion protein comprising the catalytic domain of an ADAR, or a functional portion thereof, fused to a foreign (not naturally forming part of the polypeptide chain of the ADAR) polypeptide. The foreign polypeptide may be an inactive Cas-enzyme (dead Cas9 or Cas13), a lambda phage polypeptide, or any other polypeptide that may support in, for example, binding of the editor to the target nucleic acid, the specificity of binding to the target base, or catalysis of the editing reaction). Editors may be natural editors, resident and expressed in the target cells of an individual in need of treatment. Editors may be non-naturally occurring in the target cells of the individual in need of treatment, in which case the editor must be delivered to the target cells as protein or as nucleic acid coding for the editor. Delivery of the nucleic acid coding for the editor, may be in the form of RNA, such as an mRNA (comprising all-natural nucleotides or synthetically modified nucleotides, or a mix thereof), without or with carrier (such as a nanoparticle), or in the form of an expressible construct as part of a plasmid or viral vector (such as an AAV vector). This is not essential to the present invention.

In another embodiment, the method according to the invention comprises administering a guide oligonucleotide to lead the editor (typically a Cas-enzyme or Cas-like enzyme) to the target base. In the case of DNA editing using active Cas-enzymes, the guide comprises a CRISPR sequence designed to bind to the sequence comprising the target base. In case of DNA base editing, the guide is designed to lead the editor to the target base, wherein the editor may be a combination (e.g., a fusion of two polypeptides forming a single polypeptide chain) of an inactive Cas enzyme and an active base editor, such as an adenosine deaminase or cytidine deaminase, or a portion thereof. In case of an RNA base editor the guide oligonucleotide may be an RNA guide oligonucleotide, a hybrid RNA-DNA guide oligonucleotide, and/or a synthetic guide oligonucleotide, comprising nucleotides with modified internucleosidic linkages, sugar and/or base moieties. Guide oligonucleotides may be delivered in the form of a plasmid, a DNA vector, a viral vector or otherwise, coding for the guide oligonucleotide in an expressible form, but this is not essential to the invention. According to a preferred embodiment, the editor is an ADAR, the target nucleic acid is a pre-mRNA or mRNA coding for the PCSK9 proprotein, the processing, affecting its regulation (activation or deactivation) and/or or localization, of which is to be altered, and the guide is a synthetic oligonucleotide.

In one embodiment, the present invention relates to ‘naked’ guide oligonucleotides (as is), or that are expressed from a (viral) vector. In a preferred embodiment, the guide oligonucleotides do not comprise a targeting domain that generates a loop structure for complexing with an effector protein such as those of the CRISPR system. Where RNA-editing guide oligonucleotides as disclosed in WO2016/097212 and WO2019/005884 with their internal loop structures that recruit or are linked to effector proteins may be considered as first-generation RNA editing oligonucleotides, the present invention, in a preferred embodiment, relates to RNA editing (guide) oligonucleotides that do not contain such an internal loop structure and may therefore be regarded as the second-generation RNA editing oligonucleotides. WO2019/005884 discloses RNA editing oligonucleotides for use in targeting G>A mutations in the human USH2A gene, that causes Usher Syndrome type II, a degenerative disease of the retina and inner ear. WO2019/005884 discloses RNA editing oligonucleotides that form a loop structure, and that are also complexed to a (mutated) ADAR enzyme. The present invention, in one embodiment, relates to RNA editing guide oligonucleotides that do not comprise a sequence that forms a loop structure, and that are not complexed (covalently or non-covalently) to an ADAR enzyme, or a mutated ADAR enzyme, but rather makes use of endogenous deaminase enzymes such as ADAR, which is already present in the target cell. The guide oligonucleotides in a preferred embodiment of the present invention recruit such enzymes after administration to the cell or the tissue, inside the cell, but are also capable of recruiting ADAR enzymes in biochemical assays as shown in the accompanying example(s). In a preferred embodiment, the deaminase is an ADAR enzyme, more preferably ADAR1 or ADAR2, that is already present at endogenous levels inside the target cell and does not need to be administered, such as through an expression vector, or otherwise.

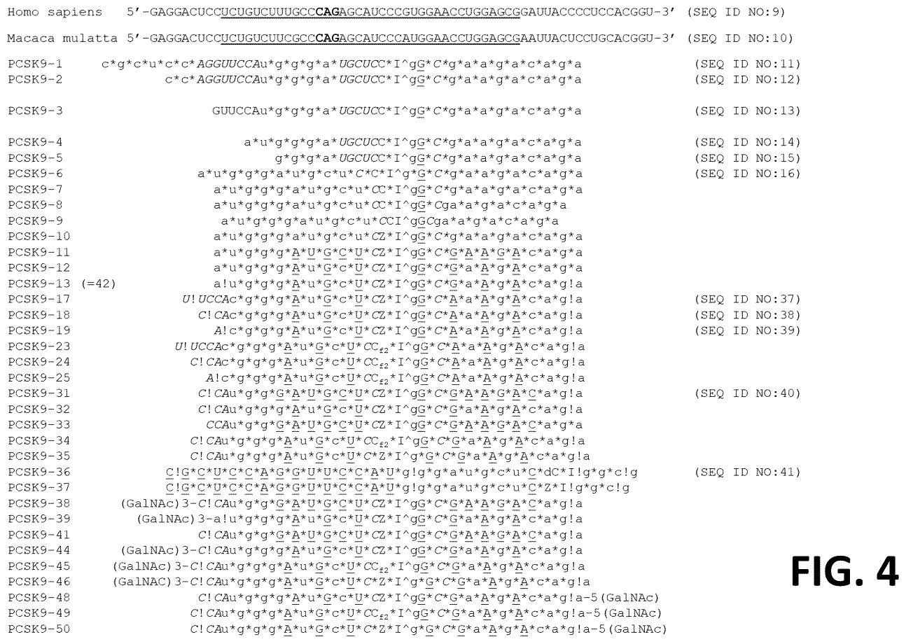

The present invention relates to an antisense RNA-editing producing oligonucleotide (EON) capable of forming a double-stranded complex with a region of an endogenous human PCSK9 transcript molecule in a cell, wherein the region of the PCSK9 transcript molecule comprises a target adenosine, and wherein the double-stranded complex can recruit an endogenous ADAR enzyme to deaminate the target adenosine into an inosine, thereby editing the PCSK9 transcript molecule. Preferably, the PCSK9 transcript molecule is a pre-mRNA or an mRNA molecule. In one embodiment, the cell is a human liver cell, preferably a hepatocyte. In one embodiment, the target adenosine is in the codon encoding glutamine at position 152 of the PCSK9 proprotein, which is deaminated to become an inosine. In one embodiment, the EON(=guide oligonucleotide) of the present invention comprises or consists of the sequence of any one of the guide oligonucleotide sequences depicted in FIG. 4. Preferably, at least one nucleotide comprises one or more non-naturally occurring chemical modifications, or one or more additional non-naturally occurring chemical modifications, in the ribose, linkage, or base moiety, with the proviso that the orphan nucleotide, which is the nucleotide in the EON that is directly opposite the target adenosine, is not a cytidine comprising a 2′-OMe ribose substitution. In one embodiment, the one or more additional modifications in the linkage moiety is each independently selected from a phosphorothioate (PS), phosphonoacetate, phosphorodithioate, methylphosphonate (MP), sulfonylphosphoramidate, or PNdmi internucleotide linkage. In one embodiment, the one or more additional modifications in the ribose moiety is a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

The present invention relates to a guide oligonucleotide for use in the treatment, delay, or prevention of hypercholesterolemia, cardiovascular disease, liver injury and/or steatohepatitis, wherein the oligonucleotide is capable of inducing editing of a nucleic acid encoding a human PCSK9 proprotein, and wherein the nucleic acid editing decreases or prevents the ability of the PCSK9 proprotein from being processed by auto-cleavage of a proteolytic cleavage site, which is preferably the P1 cleavage site. Preferably, the nucleic acid that is edited is RNA or DNA. In a preferred embodiment, the nucleic acid is RNA and the guide oligonucleotide can form a double stranded complex with a human PCSK9 pre-mRNA or mRNA, or a part thereof, wherein the guide oligonucleotide when complexed with the PCSK9 pre-mRNA or mRNA is able to engage an enzyme comprising adenosine deaminase activity, preferably an endogenous ADAR enzyme, thereby allowing the deamination of a target adenosine in the human PCSK9 pre-mRNA or mRNA. In a preferred aspect, the guide oligonucleotide comprises an orphan base that is a cytidine. Alternatively, the orphan base is a cytidine analog. The most preferred cytidine analog is the Benner's base (often abbreviated as dZ), that is disclosed in WO 2020/252376. Benner's base is also referred to as 6-amino-5-nitro-3-(1′-p-D-2′-deoxyribofuranosyl)-2(1H)-pyridone (see Yang et al. Nucl Acid Res 2006. 34(21):6095-6101). In another preferred aspect, the target adenosine is the second nucleotide of the codon coding for the glutamine residue at position 152 in the PCSK9 proprotein. In another preferred aspect, the guide oligonucleotide comprises or consists of 20 to 50 nucleotides, more preferably 23 to 27 nucleotides, and wherein the guide oligonucleotide is substantially complementary to a region within the human PCSK9 pre-mRNA or mRNA that comprises the codon coding for the glutamine residue at position 152 in the PCSK9 proprotein. For liver delivery it is preferred that the guide oligonucleotide is conjugated to a GalNAc moiety. In one embodiment, the invention relates to a guide oligonucleotide comprising the sequence of any one of the oligonucleotides displayed in FIG. 4, preferably comprising the sequence selected from the group consisting of: SEQ ID NO:11, 12, 13, 14, 15, 16, 37, 38, 39, and 40, even more preferably with a chemical modification as disclosed in FIG. 4 and as further outlined herein. In one embodiment, the invention relates to a guide oligonucleotide comprising the sequence of PCSK9-32, PCSK9-34, or PCSK9-35, as displayed in FIG. 4, more preferably wherein the guide oligonucleotide comprises the chemical modifications as detailed for any of these three guide oligonucleotides in FIG. 4. In one embodiment, the invention relates to a guide oligonucleotide comprising the sequence of PCSK9-32, PCSK9-34, or PCSK9-35, as displayed in FIG. 4, wherein the guide oligonucleotide is bound to a GalNAc moiety at the 5′ terminus or at the 3′ terminus, preferably at the 3′ terminus. More preferably the guide oligonucleotide with the attached GalNAc moiety comprises the chemical modifications as detailed for any of these three guide oligonucleotides in FIG. 4.

In a particular aspect, the invention relates to a guide oligonucleotide that comprises one or more nucleotides that comprise a modification (as further outlined in detail herein) of a nucleobase, a sugar and/or an inter-nucleosidic linkage, preferably wherein the guide oligonucleotide comprises:
at least one non-naturally occurring inter-nucleosidic linkage modification selected from the group consisting of: phosphorothioate (PS), chirally pure PS, Rp PS, Sp PS, phosphorodithioate, phosphonoacetate, thiophosphonoacetate, phosphonacetamide, thiophosphonacetamide, PS prodrug, S-alkylated PS, H-phosphonate, methyl phosphonate (MP), methyl phosphonothioate, methyl phosphate, methyl phosphorothioate, ethyl phosphate, ethyl PS, boranophosphate, boranophosphorothioate, methyl boranophosphate, methyl boranophosphorothioate, methyl boranophosphonate, methyl boranophosphonothioate, phosphorylguanidine, methyl sulfonylphosphoroamidate, phosphoramidite, phosphonamidite, N3′4P5′ phosphoramidate, N3′4P5′ thiophosphoramidate, phosphorodiamidate, phosphorothiodiamidate, sulfamate, dimethylenesulfoxide, sulfonate, triazole, oxalyl, carbamate, methyleneimino, thioacetamido, and their derivatives; and/or
at least one nucleotide that comprises a mono- or disubstitution at the 2′, 3′ and/or 5′ position of the sugar, selected from the group consisting of: —OH; —F; substituted or unsubstituted, linear or branched lower (Cl-C10) alkyl, alkenyl, alkynyl, alkaryl, allyl, or aralkyl, that may be interrupted by one or more heteroatoms; O-, S-, or N-alkyl; O-, S-, or N-alkenyl; O-, S-, or N-alkynyl; O-, S-, or N-allyl; O-alkyl-O-alkyl; -methoxy; -aminopropoxy; -methoxyethoxy; -dimethylamino oxyethoxy; and -dimethylaminoethoxyethoxy, with the proviso that the orphan base is not modified with 2′-O-methyl or 2′-methoxyethoxy.

In one embodiment, a viral vector expressing a guide oligonucleotide as characterized herein is disclosed, wherein the viral vector is for use in the treatment, delay, or prevention of hypercholesterolemia, cardiovascular disease, liver injury and/or alcohol-induced steatohepatitis, wherein the oligonucleotide can edit a nucleic acid encoding a human PCSK9 proprotein, and wherein the nucleic acid editing decreases or prevents the ability of the PCSK9 proprotein from being processed by auto-cleavage of a proteolytic cleavage site. The invention also relates to a pharmaceutical composition comprising a guide oligonucleotide as characterized herein or a viral vector as characterized herein, and a pharmaceutically acceptable carrier.

In one embodiment a method is disclosed for treating a human subject suffering from hypercholesterolemia by decreasing or inhibiting an auto-cleaving ability of the PCSK9 proprotein in a liver cell of the subject, the method comprising administering to the subject a guide oligonucleotide as characterized herein, a viral vector as characterized herein, or a pharmaceutical composition of the invention, thereby editing a nucleic acid encoding the PCSK9 proprotein. Preferably, the auto-cleavage site is the P1 cleavage site of PCSK9 and the nucleic acid is RNA or DNA. In another preferred aspect, the nucleic acid is RNA and the guide oligonucleotide can form a double stranded complex with a human PCSK9 pre-mRNA, or mRNA, or a part thereof, wherein the guide oligonucleotide when complexed with the PCSK9 pre-mRNA or mRNA is able to engage an enzyme comprising deaminase activity, thereby allowing the deamination of a target adenosine in the pre-mRNA or mRNA at the P1 cleavage site. In a preferred embodiment of the method as disclosed herein, the adenosine in the codon encoding glutamine at position 152 of the PCSK9 proprotein is deaminated to become an inosine.

In one embodiment, a method is disclosed for treating a human subject suffering from, or that is at risk of developing liver injury by decreasing or inhibiting an auto-cleaving ability of the PCSK9 proprotein in a liver cell of the subject, the method comprising administering to the subject a guide oligonucleotide as characterized herein, a viral vector as characterized herein, or a pharmaceutical composition of the invention, thereby editing a nucleic acid encoding the PCSK9 proprotein. Preferably, the auto-cleavage site is the P1 cleavage site of PCSK9 and the nucleic acid is RNA or DNA. In another preferred aspect, the nucleic acid is RNA and the guide oligonucleotide can form a double stranded complex with a human PCSK9 pre-mRNA, or mRNA, or a part thereof, wherein the guide oligonucleotide when complexed with the PCSK9 pre-mRNA or mRNA is able to engage an enzyme comprising deaminase activity, thereby allowing the deamination of a target adenosine in the pre-mRNA or mRNA at the P1 cleavage site. In a preferred embodiment of the method as disclosed herein, the adenosine in the codon encoding glutamine at position 152 of the PCSK9 proprotein is deaminated to become an inosine, thereby generating a PCSK9 protein carrying an arginine at position 152 instead of a glutamine.

In one embodiment, a method is disclosed for treating a human subject suffering from, or that is at risk of developing alcohol-induced steatohepatitis by decreasing or inhibiting an auto-cleaving ability of the PCSK9 proprotein in a liver cell of the subject, the method comprising administering to the subject a guide oligonucleotide as characterized herein, a viral vector as characterized herein, or a pharmaceutical composition of the invention, thereby editing a nucleic acid encoding the PCSK9 proprotein. Preferably, the auto-cleavage site is the P1 cleavage site of PCSK9 and the nucleic acid is RNA or DNA. In another preferred aspect, the nucleic acid is RNA and the guide oligonucleotide can form a double stranded complex with a human PCSK9 pre-mRNA, or mRNA, or a part thereof, wherein the guide oligonucleotide when complexed with the PCSK9 pre-mRNA or mRNA is able to engage an enzyme comprising deaminase activity, thereby allowing the deamination of a target adenosine in the pre-mRNA or mRNA at the P1 cleavage site. In a preferred embodiment of the method as disclosed herein, the adenosine in the codon encoding glutamine at position 152 of the PCSK9 proprotein is deaminated to become an inosine, thereby generating a PCSK9 protein carrying an arginine at position 152 instead of a glutamine.

In one embodiment, a method is disclosed for treating a human subject suffering from, or that is at risk of developing cardiovascular disease by decreasing or inhibiting an auto-cleaving ability of the PCSK9 proprotein in a liver cell of the subject, the method comprising administering to the subject a guide oligonucleotide as characterized herein, a viral vector as characterized herein, or a pharmaceutical composition of the invention, thereby editing a nucleic acid encoding the PCSK9 proprotein. Preferably, the auto-cleavage site is the P1 cleavage site of PCSK9 and the nucleic acid is RNA or DNA. In another preferred aspect, the nucleic acid is RNA and the guide oligonucleotide can form a double stranded complex with a human PCSK9 pre-mRNA, or mRNA, or a part thereof, wherein the guide oligonucleotide when complexed with the PCSK9 pre-mRNA or mRNA is able to engage an enzyme comprising deaminase activity, thereby allowing the deamination of a target adenosine in the pre-mRNA or mRNA at the P1 cleavage site. In a preferred embodiment of the method as disclosed herein, the adenosine in the codon encoding glutamine at position 152 of the PCSK9 proprotein is deaminated to become an inosine, thereby generating a PCSK9 protein carrying an arginine at position 152 instead of a glutamine.

In one embodiment a method is disclosed for treating a human subject suffering from, or being at risk of suffering from hypercholesterolemia, cardiovascular disease, liver injury, or alcohol-induced steatohepatitis by decreasing or inhibiting an auto-cleaving ability of the PCSK9 proprotein in a liver cell of the subject, the method comprising administering to the subject a guide oligonucleotide as characterized herein before, after or together with a moiety that increases the efficiency in which the guide oligonucleotide enters the cell and/or wherein the efficiency in which the guide oligonucleotide traffics through the cell, for instance because of increased endosomal release. One preferred moiety that increases such efficiency and that is preferably used in a method according to the present invention is a saponin (also referred to as a triterpene glycoside, or triterpene saponin), preferably AG1856 purified from Agrostemma githago L. as disclosed in WO2021/122998.

In another aspect disclosed is a guide oligonucleotide for the targeted deamination of a target adenosine in a target RNA, wherein the guide oligonucleotide can form a duplex with the target RNA and the duplex can recruit an enzyme comprising adenosine deaminase activity and inducing deamination of the target adenosine, wherein the guide oligonucleotide comprises a GalNAc moiety attached to it.

The enzyme comprising adenosine deaminase activity ir preferably ADAR1 or ADAR2, more preferably human ADAR1 or human ADAR2. In a preferred embodiment the guide oligonucleotide is for use in the treatment of hypercholesterolemia, cardiovascular disease, liver injury and/or alcohol-induced steatohepatitis, preferably caused by decrease of LDL receptor activity, and preferably wherein the target RNA is expressed in a liver cell, such as a hepatocyte. Preferably, the target RNA codes for a PCSK9 proprotein as further outlined herein. Even more preferably, the deamination of the target adenosine changes the glutamine codon at the P1 autocleavage site of a primate PCSK9, preferably the glutamine residue at position 152 in human PCSK9 proprotein that is then edited to an arginine.

Most of the RNA editing prior art relates to the general applicability of this phenomenon for any type of disease or genetic disorder in which a specific target adenosine should be edited to an inosine to restore translation (where the adenosine was part of a stop codon), and/or to repair the RNA when the adenosine was part of a codon that altered the protein and caused the genetic disease. The documents in the art did not specifically reveal the application of RNA editing oligonucleotides in the treatment of (familial or non-hereditary) hypercholesterolemia caused by the activity of PCSK9, or how this specifically should be performed. In contrast, a lot of prior art has accumulated that reveals the usefulness of (antisense) oligonucleotides in downregulating protein expression, or that influence splicing (e.g., see WO2012/168435, WO2013/036105, WO2016/005514, WO2016/034680, WO2016/138353, WO2016/135334, WO2017/060317, WO2017/186739, WO2018/055134, WO2015/004133, WO2018/189376, WO2018/109011, and U.S. Pat. No. 9,353,371). WO2018/154380 discloses the use of the CRISPR system to modulate the expression of PCSK9, but not specifically to alter the P1 site. WO2018/119354 discloses genome (DNA) editing of the gene encoding PCSK9 using APOBEC deaminases, but also not to edit the P1 site. To the best of the knowledge of the inventors of the present invention the use of RNA editing guide oligonucleotides has not been published for the purpose to deaminate specific adenosines in pre-mRNA or mRNA of human PCSK9, especially to deaminate an adenosine within the region encoding the P1 auto-cleavage site of PCSK9 and applying such RNA editing guide oligonucleotides in the treatment of (familial or non-hereditary) hypercholesterolemia.

The present invention, in one embodiment, relates to RNA editing guide oligonucleotides (often, and sometimes herein abbreviated to “EONs”) and their use in the treatment of cardiovascular disease, particularly by preventing high LDL cholesterol levels caused by a diminished level of LDL receptors, more preferably by inhibiting the activity or reducing the amount of active PCSK9 in liver cells. The guide oligonucleotides of the present invention preferably target the adenosine in the GAC codon encoding the glutamine at position 152 of the human PCSK9 protein, and deaminate it, through the activity of (preferably endogenous) ADAR enzymes, to an inosine read as guanosine in translation. Deamination of the adenosine to an inosine results in a disabled PCSK9 protein, unable to cause the degradation of the LDL receptor, thereby allowing the uptake of more LDL from circulation. The total effect thereof is reduced LDL plasma levels and concomitant reduction of the cardiovascular disease.

In one embodiment, the invention relates to an guide oligonucleotide capable of forming a double stranded complex with a target RNA molecule, wherein the guide oligonucleotide when complexed with the target RNA molecule, is able to recruit and complex with an ADAR enzyme, thereby allowing the deamination by the ADAR enzyme of a target adenosine in the target RNA molecule, wherein the target RNA molecule is a human PCSK9 pre-mRNA or mRNA, or a part thereof, preferably the region including the codon coding for Gln152 of the PCSK9 proprotein. It is to be noted that the guide oligonucleotide is delivered to the cell as is (naked), or through expression from a viral vector or other expression vector. Once in the cell, the guide oligonucleotide targets the PCSK9 target pre-mRNA or mRNA and hybridizes with it. The guide oligonucleotide is ‘substantially complementary’ to its target sequence which means that under physiological conditions it can form a Watson-Crick double stranded complex and that the target sequence is recognized as the opposite strand. This double stranded complex of the guide oligonucleotide and its target sequence is then able to attract (or recruit) an endogenous ADAR enzyme, which then subsequently deaminates the target adenosine, that is opposite the central nucleotide in the guide oligonucleotide, which nucleotide is preferably a cytidine. This central nucleotide is herein and often referred to as the ‘orphan base’. ‘Central’ in this context means the “0” position in the guide oligonucleotide. ‘Central’ does not mean that the guide oligonucleotide necessarily has an equal number of nucleotides upstream and downstream, although this may be the case, but simply that it is the position that sits opposite the target adenosine. This allows for specific RNA editing of the target adenosine in the human PCSK9 pre-mRNA or mRNA. The guide oligonucleotide of the present invention is not bound to or (non-) covalently attached to any proteins before it enters the cell, when administered in a ‘naked’ form. In this embodiment, it also does not form an internal loop structure to bind ADAR, or any other effector proteins, such as seen in the art for the CRISPR/Cas system. In this preferred embodiment, the guide oligonucleotide comprises an orphan base that is preferably a cytidine or a cytidine analog, which is more preferably a Benner's base (dZ). In one embodiment, the EON that is capable of forming a double stranded nucleic acid complex with a target RNA molecule, wherein the double stranded nucleic acid complex can recruit an adenosine deaminating enzyme for deamination of at least one target adenosine in the target RNA molecule, comprises a uridine analog or uridine derivative that is directly opposite the target adenosine, wherein the uridine analog or uridine derivative serves as an H-bond donor at the N3 site. Examples of preferred uridine analogs and uridine derivatives are iso-uridine, pseudouridine, 4-thiouridine, thienouridine, 5-methoxyuridine, dihydrouridine, 5-methyluridine N3-glycosylated uridine, and dihydro-iso-uridine. These uridine analogs/derivatives can come in an RNA or DNA format or can potentially be modified at the 2′ position. Other uridine analogs that can also be used in oligonucleotides according to the invention are derivatives of iso-uridine, such as substituted iso-uridine variants (with e.g., nitro, alkyl, F, Cl, Br, CN, etc.). Preferably, the ADAR enzyme that is attracted by the dsRNA complex is ADAR1 or ADAR2. If the target organ is the liver (such as is the case for the PCSK9 gene or transcript), more in particular hepatocytes, the guide oligonucleotide may conveniently be made as a conjugate comprising a GalNAc moiety attached to it. A preferred GalNAc moiety that can be applied in the teaching of the present invention is disclosed in WO2022/271806. As is well known in the art, GalNAc interacts with the asialoglycoprotein receptor abundantly present on hepatocytes, allowing the targeting of and uptake by hepatocytes of the guide oligonucleotide/GalNAc conjugate. Means of manufacturing guide oligonucleotide/GalNAc conjugates are routine for persons of skill in the art. The present inventors have shown that GalNAc-conjugated guide oligonucleotides are able to recruit ADARs and are compatible with binding and catalysis of the editing reaction. As shown in the accompanying example(s) the person skilled in the art can set up a biochemical assay that is representative for the in vivo situation in the sense that the guide oligonucleotide is brought into contact with its target sequence and recruit ADAR enzyme within the assay and bring about RNA editing of a specific target adenosine in the PCSK9 target molecule.

In a preferred embodiment, the guide oligonucleotide comprises a non-naturally occurring chemical modification, with the proviso that the orphan base does not have a 2′-O-methyl (2′-OMe) or a 2′-methoxyethoxy (2′-MOE) modification in the sugar moiety. Preferably, the modification of the orphan base (or nucleotide) is selected from the group consisting of: deoxyribose (DNA), Unlocked Nucleic Acid (UNA) and 2′-fluororibose. As outlined further herein, DNA is then considered to be a chemical derivative of RNA. Most (but in some embodiments certainly not all) nucleotides within a guide oligonucleotide of the present invention is RNA, that may be modified with non-naturally occurring substituents as detailed further herein. For instance, in a particular embodiment, at least the two, three, four, five, or six terminal nucleotides of the 5′ and 3′ terminus of the guide oligonucleotide are linked with phosphorothioate (PS) linkages, preferably wherein the terminal five nucleotides at the 5′ and 3′ terminus are linked with PS linkages. Further nucleotides more towards the orphan base may also be connected by a non-naturally occurring linkage, such as PS. In another preferred embodiment, the guide oligonucleotide is chemically modified to render the guide oligonucleotide stable towards breakdown by the cellular RNases and other environmental circumstances in vivo. For this, it is preferred that one or more nucleotides in the guide oligonucleotide comprise a mono- or disubstitution at the 2′, 3′ and/or 5′ position of the sugar.

RNA editing according to the system as outlined by the present invention, preferably targets a single adenosine in a target molecule (a pre-mRNA or mRNA transcribed of the human PCSK9 gene). Preferably, the target adenosine is part of a codon encoding glutamine at position 152 of the PCSK9 protein. In another preferred embodiment, the target adenosine is part of any other codon involved in the activity of the PCSK9 protein. In other words, even though the invention is exemplified by altering the auto-cleavage activity of PCSK9 by targeting Gln152, it may be that other amino acids are involved in this process or are key in the activity of PCSK9, and that may be altered through RNA editing as well, separately, or together with RNA editing of the adenosine in the Gln152 codon. It is to be understood that a gain-of-function mutation of PCSK9 may also be targeted through RNA editing when this gain-of-function mutation contains (in its mRNA) an adenosine that—when deaminated—loses the gain-of-function.

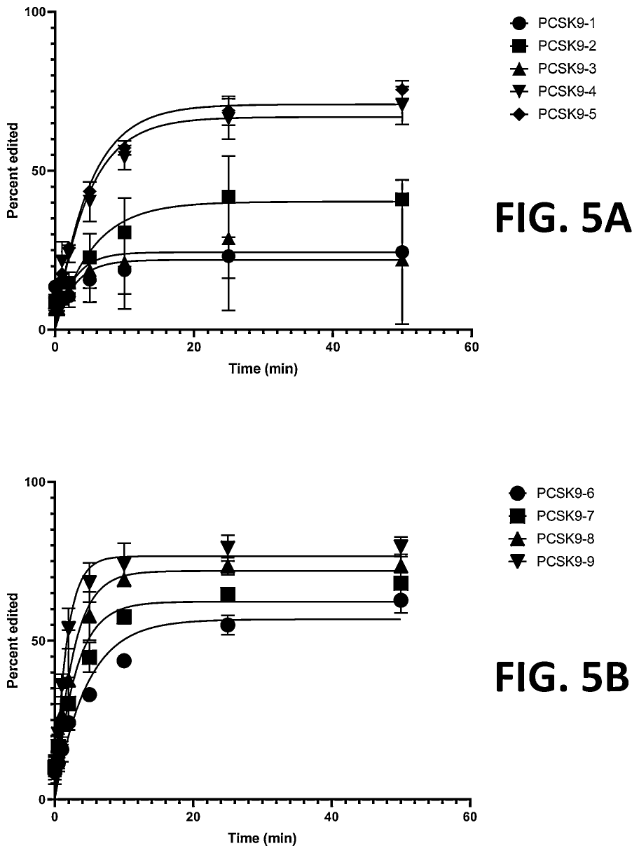

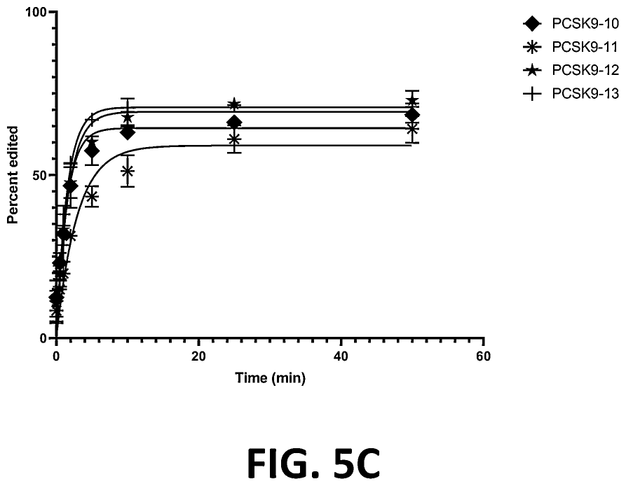

In a further embodiment, the invention relates to a guide oligonucleotide that targets a human PCSK9 (pre-) mRNA target molecule, or a part thereof that includes the adenosine within the codon for Gln152 (CAG) of PCSK9, such as the part given in FIG. 4. Preferably, the invention relates to a guide oligonucleotide that comprises or consists of the sequence of PCSK9-1, -2, -3, -4, -5, -6, -7, -8, -9, -10, -11, -12, -13, -31, -32, -33, -34, -35, -36, -37, -38, -39, -41, -42, -44, -45, -46, -48, 49, -50, -17, -18, -19, -23, -24, or -25 in FIG. 4. In a further preferred aspect, the guide oligonucleotides comprise the modifications for each of these oligonucleotides as indicated in FIG. 4—and the legend of FIG. 4. In another preferred embodiment, the guide oligonucleotide according to the invention comprises at least one MP linkage, preferably at linkage position −1, following the nucleotide and linkage numbering as disclosed in FIG. 5 of WO 2020/201406. The at least one MP internucleoside linkage is according to the following structure:

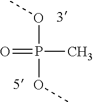

In one embodiment, the EON comprises at least one phosphonoacetate or phosphonoacetamide internucleoside linkage.

The invention also relates to a composition comprising a set of two oligonucleotides (AONs), wherein one AON is a guide oligonucleotide according to the invention, and the other AON is the ‘Helper AON’, for use in the deamination of a target adenosine in a human PCSK9 pre-mRNA or mRNA, or a part thereof, wherein the Helper AON is complementary to a stretch of nucleotides in the human PCSK9 pre-mRNA or mRNA that is separate from the stretch of nucleotides that is complementary to the guide oligonucleotide, wherein the Helper AON has a length of 16 to 22 nucleotides and the guide oligonucleotide has a length of 16 to 22 nucleotides.

The invention also relates to Heteroduplex RNA Editing Oligonucleotide (HEON) complexes as described in GB 2215614.5 (unpublished) for targeting PCSK9 (pre-) mRNA according to the present invention. Hence, the invention also relates to a HEON for use in the deamination of a target adenosine present in a target PCSK9 RNA molecule in a cell, wherein the HEON comprises a first nucleic acid strand that is annealed to a complementary second nucleic acid strand, wherein the first nucleic acid strand is complementary to a stretch of nucleotides in the PCSK9 target RNA molecule that includes the target adenosine, wherein the first nucleic acid strand when it is hybridized to the target RNA molecule is capable of recruiting an enzyme with deaminase activity present in the cell allowing for the deamination of the target adenosine, and wherein the first and/or second nucleic acid strand is bound to a hydrophobic moiety. The first nucleic acid strand is the ‘active’ or ‘guide’ oligonucleotide targeting the PCSK9 transcript as disclosed herein. Preferably, the hydrophobic moiety is a lipid, a hydrophobic vitamin, or a steroid. More preferably, the hydrophobic vitamin is vitamin E or analog thereof, and the steroid is cholesterol or analog thereof. In a preferred embodiment, the hydrophobic moiety is bound to the 5′ terminus, to the 3′ terminus, and/or to an internal position of the first nucleic acid strand and/or of the second nucleic acid strand. In a preferred embodiment, the HEON according to the invention is further bound to a ligand that enables cell-specific targeting, such as a GalNAc moiety. In a preferred embodiment, the nucleotide in the first nucleic acid strand that is directly opposite the target adenosine is the orphan nucleotide that has the structure of formula I:

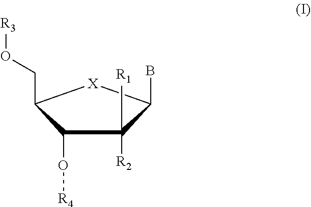

wherein: X is O, NH, OCH₂, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-3-(1′-p-D-2′-deoxyribofuranosyl)-2(1H)-pyridone; R₁ and R₂ are both selected, independently, from H, OH, F or CH₃; R₃ is the part of the first nucleic acid strand that is 5′ of the orphan nucleotide, consisting of 7 to 30 nucleotides; and R₄ is the part of the first nucleic acid strand that is 3′ of the orphan nucleotide, consisting of 4 to 25 nucleotides. In a preferred embodiment, the first nucleic acid strand and/or the second nucleic acid strand comprises at least one phosphorothioate (PS), phosphonoacetate, methylphosphonate (MP), phosphoryl guanidine, or PNdmi internucleotide linkage. Isouracil is a uracil analog as disclosed in WO2021/071858. A PNdmi linkage has the structure of the following formula:

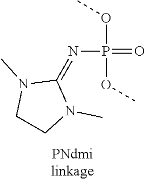

In a preferred embodiment, the HEON comprising the PCSK9 targeting oligonucleotide as the first nucleic acid strand according to the present invention and as outlined herein is for use in the treatment or prevention of liver injury, alcohol-induced steatohepatitis, cardiovascular disease, more preferably hypercholesterolemia, even more preferably caused by the diminished levels (or activity) of LDL receptors, and even more preferably wherein this diminished level or diminished activity is mediated by PCSK9.

The invention further relates to a guide oligonucleotide according to the invention for use in the treatment of liver injury, alcohol-induced steatohepatitis, cardiovascular disease, more preferably hypercholesterolemia, even more preferably caused by the diminished levels (or activity) of LDL receptors, and even more preferably wherein this diminished level or diminished activity is mediated by PCSK9. The PCSK9 may be wild-type or comprise a gain-of-function mutation that causes increased intracellular endosomal or lysosomal breakdown of LDL receptors.

The invention further relates to the use of a guide oligonucleotide according to the invention for the manufacture of a medicament for use in the treatment, amelioration, prevention or slowing down the progression of liver injury, alcohol-induced steatohepatitis, cardiovascular disease, more preferably hypercholesterolemia, even more preferably caused by the diminished levels (or activity) of LDL receptors, and even more preferably wherein this diminished level or diminished activity is mediated by PCSK9. The PCSK9 may be wild-type or comprise a gain-of-function mutation that causes increased intracellular endosomal or lysosomal breakdown of LDL receptors.

The invention further relates to a method for the deamination of at least one specific target adenosine present in a target RNA molecule in a cell, wherein the target RNA molecule is a human PCSK9 pre-mRNA or mRNA, or a part thereof, the method comprising the steps of: providing the cell with a guide oligonucleotide according to the invention; allowing uptake by the cell of the guide oligonucleotide; allowing annealing of the guide oligonucleotide to the target RNA molecule; allowing a mammalian ADAR enzyme comprising a natural dsRNA binding domain as found in the wild type enzyme to deaminate the target adenosine in the target RNA molecule to an inosine; and optionally identifying the presence of the inosine in the target RNA molecule. The optional step preferably comprises: (i) sequencing the target RNA molecule; (ii) assessing the presence or absence of a functional, full length and/or wild type cleaved PCSK9; (iii) assessing whether splicing of the pre-mRNA was modulated by the deamination; or (iv) using a functional read-out, wherein LDL levels are determined (in vivo) or in tissue or plasma samples. Because the gene encoding human PCSK9 and rhesus monkey PCSK9 are well conserved, such activity of the PCSK9 protein may be assessed properly in this species. The advantage here is also that the wild type (pre-) mRNA may be targeted, which means that there is no need for animal studies wherein the animals carry a mutated PCSK9 gene.

The invention further relates to a method of treating, ameliorating, preventing and/or slowing down the progression of cardiovascular disease in a human subject in need thereof that is suffering from, or is at risk of suffering from, cardiovascular disease, comprising the step of administering a guide oligonucleotide according to the invention, or a pharmaceutical composition according to the invention, to the human subject. In a preferred embodiment, the guide oligonucleotide is administered to the human subject in need thereof, by systemic or local administration, preferably directly into the liver to target hepatocytes, preferably through direct injection of the naked (and chemically modified) guide oligonucleotide and allowing the targeting of a wild type PCSK9 pre-mRNA or gain-of-function mutant PCSK9 mRNA in a liver cell. Preferably the guide oligonucleotide of the present invention is chemically modified to increase the uptake of the guide oligonucleotide by liver cells, more in particular hepatocytes, such as through the conjugation of N-acetylgalactosamine (GalNAc) moieties that bind to the asialoglycoprotein receptor (ASGPR) that are well known in the art.

A guide oligonucleotide of the present invention does not have to comprise a recruitment portion as described in WO2016/097212. The guide oligonucleotides of the present invention do not have to comprise a portion that can form an intramolecular stem-loop structure. The preferred guide oligonucleotides of the present invention are shorter than those forming a (ADAR recruiting) loop structure, which makes them cheaper to produce, easier to use and easier to manufacture. Furthermore, they are likely to enter cells more efficiently than longer oligonucleotides and are less prone to degradation. WO2017/220751 and WO2018/041973 disclose EONs that are complementary to a target RNA for deaminating a target adenosine present in a target RNA sequence to which the EON is complementary, lacking a recruitment portion while still being capable of harnessing ADAR enzymes present in the cell to edit the target adenosine. The present invention makes use of that knowledge and aims to solve the problem of targeting cardiovascular disease, by inhibiting the activity of PCSK9, through diminishing its possibility to self-cleave.

A guide oligonucleotide of the present invention comprises one or more nucleotides with one or more non-naturally occurring sugar modifications. Thereby, a single nucleotide of the guide oligonucleotide can have one, or more than one such sugar modification. Within the guide oligonucleotide, one or more nucleotide(s) can have such sugar modification(s). It is also an aspect of the invention that the nucleotide (the orphan base) within the guide oligonucleotide of the present invention that is opposite to the nucleotide that needs to be edited does not contain a 2′-O-methyl (2′-OMe) or a 2′-methoxyethoxy (2′-MOE) modification. Often the nucleotides that are directly 3′ and 5′ of this nucleotide (the ‘neighbouring nucleotides’) in the guide oligonucleotide also lack such a chemical modification, although it is not mandatory that both neighbouring nucleotides should not contain a 2′-O-alkyl group (such as a 2′-OMe). Either one, both, or all three nucleotides of the orphan base and its two neighbouring nucleotides may carry 2′-OH.

In one embodiment, the EON is an antisense oligonucleotide that can form a double stranded nucleic acid complex with a target RNA molecule, wherein the double stranded nucleic acid complex can recruit an adenosine deaminating enzyme for deamination of a target adenosine in the target PCSK9 RNA molecule, wherein the nucleotide in the EON that is opposite the target adenosine is the orphan nucleotide, and wherein the orphan nucleotide has the following structure:

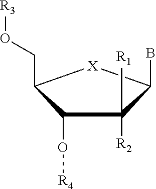

wherein: X is O, NH, OCH₂, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-3-(1′-p-D-2′-deoxyribofuranosyl)-2(1H)-pyridone; R₁ and R₂ are both selected, independently, from H, OH, F or CH₃; R₃ is the part of the EON that is 5′ of the orphan nucleotide, consisting of 7 to 30 nucleotides; and R₄ is the part of the EON that is 3′ of the orphan nucleotide, consisting of 4 to 25 nucleotides.

The skilled person knows that an oligonucleotide, such as an RNA oligonucleotide, generally consists of repeating monomers. Such a monomer is most often a nucleotide or a nucleotide analogue. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a “scaffold modification”. For severe modifications, the original pentose sugar might be replaced in its entirety by another moiety that similarly connects the base and the phosphate. It is therefore understood that while a pentose sugar is often a scaffold, a scaffold is not necessarily a pentose sugar.

A base, sometimes called a nucleobase, is generally adenine, cytosine, guanine, thymine or uracil, or a derivative thereof. Cytosine, thymine, and uracil are pyrimidine bases, and are generally linked to the scaffold through their 1-nitrogen. Adenine and guanine are purine bases and are generally linked to the scaffold through their 9-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked monomers of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate, such a moiety is still referred to as the backbone linkage of the monomer. This is referred to as a “backbone linkage modification”. In general terms, the backbone of an oligonucleotide comprises alternating scaffolds and backbone linkages.

In one embodiment, the nucleobase in a guide oligonucleotide of the present invention is adenine, cytosine, guanine, thymine, or uracil. In another embodiment, the nucleobase is a modified form of adenine, cytosine, guanine, or uracil. The nucleobases at any position in the nucleic acid strand can be a modified form of adenine, cytosine, guanine, or uracil, such as hypoxanthine (the nucleobase in inosine), pseudouracil, pseudocytosine, isouracil, N3-glycosylated uracil, 1-methylpseudouracil, orotic acid, agmatidine, lysidine, 2-thiouracil, 2-thiothymine, 5-substituted pyrimidine (e.g., 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine), 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, 3-deazapurine (such as a 3-deaza-adenosine), pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp and its derivatives, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar. The term ‘nucleoside’ refers to the nucleobase linked to the (deoxy)ribosyl sugar. The term ‘nucleotide’ refers to the respective nucleobase-(deoxy)ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group, well known in the art), a nucleotide including a linker comprising a phosphodiester, phosphotriester, phosphoro(di)thioate, methylphosphonates, phosphoramidate linkers, and the like. Sometimes the terms adenosine and adenine, guanosine and guanine, cytosine and cytidine, uracil and uridine, thymine and thymidine, inosine, and hypoxanthine, are used interchangeably to refer to the corresponding nucleobase, nucleoside, or nucleotide. Sometimes the terms nucleobase, nucleoside and nucleotide are used interchangeably, unless the context clearly requires differently.

In one embodiment, a guide oligonucleotide of the present invention comprises a 2′-substituted phosphorothioate monomer, preferably a 2′-substituted phosphorothioate RNA monomer, a 2′-substituted phosphate RNA monomer, or comprises 2′-substituted mixed phosphate/phosphorothioate monomers. It is noted that DNA is considered as an RNA derivative in respect of 2′ substitution. A guide oligonucleotide of the present invention comprises at least one 2′-substituted RNA monomer connected through or linked by a phosphorothioate or phosphate backbone linkage, or a mixture thereof. The 2′-substituted RNA preferably is 2′-F, 2′,2′-difluoro (diF), 2′-H (DNA), 2′-O-Methyl or 2′-0-(2-methoxyethyl). The 2′-O-Methyl is often abbreviated to “2′-OMe” and the 2′-O-(2-methoxyethyl) moiety is often abbreviated to “2′-MOE”. More preferably, the 2′-substituted RNA monomer in the guide oligonucleotide of the present invention is a 2′-OMe monomer, except for the monomer opposite the target adenosine, as further outlined herein, which should not carry a 2′-OMe substitution. In a preferred embodiment of this aspect is provided a guide oligonucleotide according to the invention, wherein the 2′-substituted monomer can be a 2′-substituted RNA monomer, such as a 2′-F monomer, a 2′-NH₂ monomer, a 2′-H monomer (DNA), a 2′-O-substituted monomer, a 2′-OMe monomer or a 2′-MOE monomer or mixtures thereof. Preferably, the monomer opposite the target adenosine is a 2′-H monomer (DNA) but may also be a monomer that allows deamination of the target adenosine, other than a 2′-OMe monomer. Preferably, any other 2′-substituted monomer within the guide oligonucleotide is a 2′-substituted RNA monomer, such as a 2′-OMe RNA monomer or a 2′-MOE RNA monomer, which may also appear within the guide oligonucleotide in combination.

Throughout the application, a 2′-OMe monomer within a guide oligonucleotide of the present invention may be replaced by a 2′-OMe phosphorothioate RNA, a 2′-OMe phosphate RNA or a 2′-OMe phosphate/phosphorothioate RNA. Throughout the application, a 2′-MOE monomer may be replaced by a 2′-MOE phosphorothioate RNA, a 2′-MOE phosphate RNA or a 2′-MOE phosphate/phosphorothioate RNA. Throughout the application, an oligonucleotide consisting of 2′-OMe RNA monomers linked by or connected through phosphorothioate, phosphate or mixed phosphate/phosphorothioate backbone linkages may be replaced by an oligonucleotide consisting of 2′-OMe phosphorothioate RNA, 2′-OMe phosphate RNA or 2′-OMe phosphate/phosphorothioate RNA. Throughout the application, an oligonucleotide consisting of 2′-MOE RNA monomers linked by or connected through phosphorothioate, phosphate or mixed phosphate/phosphorothioate backbone linkages may be replaced by an oligonucleotide consisting of 2′-MOE phosphorothioate RNA, 2′-MOE phosphate RNA or 2′-MOE phosphate/phosphorothioate RNA.

In addition to the specific preferred chemical modifications at certain positions in compounds of the invention, compounds of the invention may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-O-methyl, 2′-O-(2-cyanoethyl), 2′-MOE, 2′-0-(2-thiomethyl)ethyl, 2′-O-butyryl, 2′-O-propargyl, 2′-O-allyl, 2′-O-(2-aminopropyl), 2′-0-(2-(dimethylamino)propyl), 2′-O-(2-amino)ethyl, 2′-O-(2-(dimethylamino)ethyl); 2′-deoxy (DNA); 2′-O-(haloalkyl)methyl such as 2′-O-(2-chloroethoxy)methyl (MCEM), 2′-0-(2,2-dichloroethoxy)methyl (DCEM); 2′-O-alkoxycarbonyl such as 2′-O-[2-(methoxycarbonyl)ethyl](MOCE), 2′-O-[2-N-methylcarbamoyl)ethyl](MCE), 2′-O-[2-(N,N-dimethylcarbamoyl)ethyl](DCME); 2′-halo e.g. 2′-F, 2′,2′-difluoro (diF), FANA; 2′-O-[2-(methylamino)-2-oxoethyl](NMA); a bicyclic or bridged nucleic acid (BNA) scaffold modification such as a conformationally restricted nucleotide (CRN) monomer, a locked nucleic acid (LNA) monomer, a xylo-LNA monomer, an a-LNA monomer, an a-L-LNA monomer, a p-D-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-O,4′-C) constrained ethyl (cEt) BNA monomer, a (2′-0,4′-C) constrained methoxyethyl (cMOE) BNA monomer, a 2′,4′-BNANc(NH) monomer, a 2′,4′-BNA^{NC}(NMe) monomer, a 2′,4′-BNA^{NC}(NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All these modifications are known to the person skilled in the art.

A “backbone modification” indicates the presence of a modified version of the ribosyl moiety (“scaffold modification”), as indicated above, and/or the presence of a modified version of the phosphodiester as naturally occurring in RNA (“backbone linkage modification”). Examples of internucleoside linkage modifications are phosphorothioate (PS), chirally pure PS, Rp PS, Sp PS, phosphorodithioate (PS2), phosphonoacetate (PACE), thiophosphonoacetate, phosphonacetamide (PACA), thiophosphonacetamide, phosphorothioate prodrug, S-alkylated phosphorothioate, H-phosphonate, methyl phosphonate (MP), methyl phosphonothioate, methyl phosphate, methyl phosphorothioate, ethyl phosphate, ethyl PS, boranophosphate, boranophosphorothioate, methyl boranophosphate, methyl boranophosphorothioate, methyl boranophosphonate, methyl boranophosphonothioate, phosphoryl guanidine (PGO), methylsulfonyl phosphoroamidate, phosphoramidite, phosphonamidite, N3′→P5′ phosphoramidate, N3′→P5′ thiophosphoramidate, phosphorodiamidate, phosphorothiodiamidate, sulfamate, dimethylenesulfoxide, sulfonate, triazole, oxalyl, carbamate, methyleneimino (MMI), and thioacetamido (TANA); and their derivatives.

Preferred guide oligonucleotides of the present invention do not include a 5′-terminal O6-benzylguanosine or a 5′-terminal amino modification and are not covalently linked to a SNAP-tag domain (an engineered O6-alkylguanosine-DNA-alkyl transferase). In one embodiment, a guide oligonucleotide of the present invention comprises 0, 1, 2 or 3 wobble base pairs with the target sequence, and/or 0, 1, 2, or 3 mismatches with the target RNA sequence, wherein a single mismatch may comprise multiple sequential nucleotides. Similarly, a preferred guide oligonucleotide of the present invention does not include a boxB RNA hairpin sequence. A guide oligonucleotide according to the present invention can utilise endogenous cellular pathways and naturally available ADAR enzymes to specifically edit a target adenosine in a target RNA sequence. A guide oligonucleotide of the invention can ‘recruit’, or ‘lead’, or ‘guide’ ADAR and complex with it and then allow the deamination of a (single) specific target adenosine nucleotide in a target RNA sequence. Ideally, only one adenosine is deaminated. Alternatively, 1, 2, or 3 adenosine nucleotides are deaminated, for instance when target adenosines are in proximity of each other. For example, when the mutation is an alteration from a wild type GGA (glycine) codon to a mutant GAA (glutamic acid) codon, deamination of both adenosines would result in GGG, which also encodes a glycine. A guide oligonucleotide of the invention, when complexed to ADAR, preferably deaminates a single target adenosine.

Analysis of natural targets of ADAR enzymes has indicated that these generally include mismatches between the two strands that form the RNA helix edited by ADAR1 or 2. It has been suggested that these mismatches enhance the specificity of the editing reaction (Stefl et al. 2006. Structure 14(2):345-355; Tian et al. 2011. Nucleic Acids Res 39(13):5669-5681). Characterization of optimal patterns of paired/mismatched nucleotides between the guide oligonucleotides and the target RNA also appears crucial for development of efficient ADAR-based guide oligonucleotide therapy.

A guide oligonucleotide of the present invention makes use of specific nucleotide modifications at predefined spots to ensure stability as well as proper ADAR binding and activity. These changes may vary and may include modifications in the backbone of the guide oligonucleotide, in the sugar moiety of the nucleotides as well as in the nucleobases or the phosphodiester linkages, as outlined in detail above. They may also be variably distributed throughout the sequence of the guide oligonucleotide. Specific modifications may be needed to support interactions of different amino acid residues within the RNA-binding domains of ADAR enzymes, as well as those in the deaminase domain. For example, PS linkages between nucleotides or 2′-OMe or 2′-MOE modifications may be tolerated in some parts of the guide oligonucleotide, while in other parts they should be avoided so as not to disrupt crucial interactions of the enzyme with the phosphate and 2′-OH groups. Specific nucleotide modifications may also be necessary to enhance the editing activity on substrate RNAs where the target sequence is not optimal for ADAR editing. Previous work has established that certain sequence contexts are more amenable to editing. For example, the target sequence 5′-UAG-3′ (with the target A in the middle) contains the most preferred nearest-neighbor nucleotides for ADAR2, whereas a 5′-CAA-3′ target sequence is disfavored (Schneider et al. 2014. Nucleic Acids Res 42(10):e87).

Whenever herein the order of nucleotides within the guide oligonucleotide is discussed, it is always from 5′ to 3′ of the guide oligonucleotide. The position can also be expressed in terms of a particular nucleotide within the guide oligonucleotide while still adhering to the 5′ to 3′ directionality, in which case other nucleotides 5′ of the said nucleotide are marked as negative positions and those 3′ of it as positive positions.

As outlined herein, the nucleotides away from the orphan base and its two neighbouring nucleotides are often 2′-OMe or 2′-MOE modified. However, this is not a strict requirement of the guide oligonucleotides of the present invention. These 2′ substitutions ensure a proper stability of those parts of the guide oligonucleotide, but other modifications may be applied as well.

A guide oligonucleotide according to the invention may be indirectly administrated using suitable means known in the art. It may for example be provided to an individual or a cell, tissue, or organ of said individual in the form of an expression vector wherein the expression vector encodes a transcript comprising the sequence of the guide oligonucleotide. The expression vector is preferably introduced into a cell, tissue, organ, or individual via a gene delivery vehicle. In a preferred embodiment, there is provided a viral-based expression vector comprising an expression cassette or a transcription cassette that drives expression or transcription of a guide oligonucleotide as identified herein. Accordingly, the invention provides a viral vector that can express a guide oligonucleotide according to the invention (then without non-natural chemical modifications) when placed under conditions conducive to expression of the guide oligonucleotide. A cell can be provided with a guide oligonucleotide by plasmid-derived guide oligonucleotide expression or viral expression provided by adenovirus- or adeno-associated virus-based vectors. Expression may be driven by a polymerase II-promoter (Pol II) such as a U7 promoter or a polymerase III (Pol Ill) promoter, such as a U6 RNA promoter. A preferred delivery vehicle is AAV, or a retroviral vector such as a lentivirus vector and the like. Also, plasmids, artificial chromosomes, plasmids usable for targeted homologous recombination and integration in the human genome of cells may be suitably applied for delivery of a guide oligonucleotide as defined herein. Preferred for the current invention are those vectors wherein transcription is driven from Pol III promoters, and/or wherein transcripts are in the form fusions with U1 or U7 transcripts, which yield good results for delivering small transcripts. It is within the skill of the artisan to design suitable transcripts. Preferred are Pol Ill driven transcripts, preferably, in the form of a fusion transcript with an U1 or U7 transcript, known to the person skilled in the art.

Typically, when the guide oligonucleotide is delivered by a viral vector, it is in the form of an RNA transcript that comprises the sequence of an oligonucleotide according to the invention in a part of the transcript. The resulting guide oligonucleotide that is active in the cell is then not chemically modified because it is naturally expressed, while a guide oligonucleotide that is manufactured in a ‘naked’ form (=without the use of a plasmid or viral vector expressing the guide oligonucleotide) may comprise single or multiple non-naturally occurring modifications. An AAV vector according to the invention is a recombinant AAV vector and refers to an AAV vector comprising part of an AAV genome comprising an encoded guide oligonucleotide according to the invention encapsidated in a protein shell of capsid protein derived from an AAV serotype. Part of an AAV genome may contain the inverted terminal repeats (ITR) derived from an adeno-associated virus serotype, such as AAV1, AAV2, AAV3, AAV4, AAV5, AAV6, AAV7, AAV8, AAV9 and others. Protein shell comprised of capsid protein may be derived from an AAV serotype such as AAV1, 2, 3, 4, 5, 6, 7, 8, 9 and others. A protein shell may also be named a capsid protein shell. An AAV vector may have one or preferably all wild type AAV genes deleted but may still comprise functional ITR nucleic acid sequences. Functional ITR sequences are necessary for the replication, rescue, and packaging of AAV virions. The ITR sequences may be wild type sequences or may have at least 80%, 85%, 90%, 95, or 100% sequence identity with wild type sequences or may be altered by for example in insertion, mutation, deletion, or substitution of nucleotides, if they remain functional. In this context, functionality refers to the ability to direct packaging of the genome into the capsid shell and then allow for expression in the host cell to be infected or target cell. In the context of the invention a capsid protein shell may be of a different serotype than the AAV vector genome ITR. An AAV vector according to present the invention may thus be composed of a capsid protein shell, i.e., the icosahedral capsid, which comprises capsid proteins (VP1, VP2, and/or VP3) of one AAV serotype, e.g., AAV serotype 2, whereas the ITRs sequences contained in that AAV2 vector may be any of the AAV serotypes described above, including an AAV2 vector. An “AAV2 vector” thus comprises a capsid protein shell of AAV serotype 2, while e.g., an “AAV5 vector” comprises a capsid protein shell of AAV serotype 5, whereby either may encapsidate any AAV vector genome ITR according to the invention. Preferably, a recombinant AAV vector according to the invention comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

More preferably, a recombinant AAV vector according to the invention comprises a capsid protein shell of AAV serotype 2 and the AAV genome or ITRs present in said vector are derived from AAV serotype 5; such vector is referred to as an AAV 2/5 vector. More preferably, a recombinant AAV vector according to the invention comprises a capsid protein shell of AAV serotype 2 and the AAV genome or ITRs present in said vector are derived from AAV serotype 8; such vector is referred to as an AAV 2/8 vector. More preferably, a recombinant AAV vector according to the invention comprises a capsid protein shell of AAV serotype 2 and the AAV genome or ITRs present in said vector are derived from AAV serotype 9; such vector is referred to as an AAV 2/9 vector. More preferably, a recombinant AAV vector according to the invention comprises a capsid protein shell of AAV serotype 2 and the AAV genome or ITRs present in said vector are derived from AAV serotype 2; such vector is referred to as an AAV 2/2 vector. A nucleic acid molecule encoding a guide oligonucleotide according to the invention represented by a nucleic acid sequence of choice is preferably inserted between the AAV genome or ITR sequences as identified above, for example an expression construct comprising an expression regulatory element operably linked to a coding sequence and a 3′ termination sequence. “AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art. The AAV helper functions can be supplied on an AAV helper construct, which may be a plasmid.

Introduction of the helper construct into the host cell can occur e.g., by transformation, transfection, or transduction prior to or concurrently with the introduction of the AAV genome present in the AAV vector as identified herein. The AAV helper constructs of the invention may thus be chosen such that they produce the desired combination of serotypes for the AAV vector's capsid protein shell on the one hand and for the AAV genome present in said AAV vector replication and packaging on the other hand. “AAV helper virus” provides additional functions required for AAV replication and packaging.

Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456. Preferably, an AAV genome as present in a recombinant AAV vector according to the invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g., gfp) or a gene encoding a chemically, enzymatically, or otherwise detectable and/or selectable product (e.g., lacZ, aph, etc.) known in the art. A preferred AAV vector according to the invention is an AAV vector, preferably an AAV2/5, AAV2/8, AAV2/9 or AAV2/2 vector, expressing a guide oligonucleotide according to the invention.

In the present invention the target (pre-) mRNA is predominantly expressed in liver cells. To enter liver cells, very specific modifications may be applied and form embodiments of the present invention. For instance, to target hepatocytes often the ASGPR is being used for targeting and uptake, using a GalNAc moiety attached to the guide oligonucleotide. Alternatively, functionalized nanoparticles may be used that specifically or preferentially target the liver, more in particular the hepatocytes. The present inventors have shown that the GalNAc conjugate is compatible with ADAR-engagement and catalysis, using a biochemical editing assay involving human ADAR.

To the inventor's knowledge, this was not shown before to be feasible. GalNAc is a generic term that may encompass, mono, di- or tertiary antennary structures.

Whenever reference is made to a ‘guide oligonucleotide’, an ‘antisense oligonucleotide’ (‘AON’), an ‘RNA editing oligonucleotide’ (‘EON’), an ‘oligonucleotide’, or an ‘oligo’ then both oligoribonucleotides and deoxyoligoribonucleotides are meant unless the context dictates otherwise. Whenever reference is made to an ‘oligoribonucleotide’ it may comprise the ribonucleosides adenosine (A), guanosine (G), cytidine (C), 5-methylcytidine (m⁵C), uridine (U), 5-methyluridine (m⁵U) or inosine (I). Whenever reference is made to a ‘deoxyoligoribonucleotide’ it may comprise the deoxyribonucleosides deoxyadenosine (A), deoxyguanosine (G), deoxycytidine (C), thymine (T) or deoxyinosine (I). In a preferred aspect, the guide oligonucleotide of the present invention is mostly an oligoribonucleotide that may comprise chemical modifications and, at a few specified positions, deoxyribonucleosides (DNA). When reference is made to nucleotides in the oligonucleotide construct, such as cytosine, then 5-methylcytosine, 5-hydroxymethylcytosine, pyrrolocytidine, and β-D-glucosyl-5-hydroxy-methylcytosine are included. When reference is made to adenine, then 2-aminopurine, 2,6-diaminopurine, 3-deazaadenosine, 7-deazaadenosine, 8-azidoadenosine, 8-methyladenosine, 7-aminomethyl-7-deazaguanosine, 7-deazaguanosine, N6-Methyladenine and 7-methyladenine are included. When reference is made to uracil, then 5-methoxyuracil, 5-methyluracil, dihydrouracil, pseudouracil, and thienouracil, dihydrouracil, 4-thiouracil and 5-hydroxymethyluracil are included. When reference is made to guanosine, then 7-methylguanosine, 8-aza-7-deazaguanosine, thienoguanosine and 1-methylguanosine are included. When reference is made to nucleosides or nucleotides, then ribofuranose derivatives, such as 2′-deoxy, 2′-hydroxy, 2-fluororibose and 2′-O-substituted variants, such as 2′-O-methyl, are included, as well as other modifications, including 2′-4′ bridged variants.

The term “comprising” encompasses “including” as well as “consisting of”, e.g., a composition ‘comprising X’ may consist exclusively of X or may include something additional, e.g., X+Y. The term ‘about’ in relation to a numerical value x is optional and means, e.g., x±10%. The word ‘substantially’ does not exclude ‘completely’, e.g., a composition which is ‘substantially free from Y’ may be completely free from Y. Where relevant, the word ‘substantially’ may be omitted from the definition of the invention. The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand. References to ‘hybridisation’ typically refer to specific hybridisation and exclude non-specific hybridisation. Specific hybridisation can occur under experimental conditions chosen, using techniques well known in the art, to ensure that most stable interactions between probe and target are where the probe and target have at least 70%, preferably at least 80%, more preferably at least 90% sequence identity. The term ‘mismatch’ is used herein to refer to opposing nucleotides in a double stranded RNA complex which do not form perfect base pairs according to the Watson-Crick base pairing rules. Mismatch base pairs are G-A, C-A, U-C, A-A, G-G, C-C, U-U base pairs. In some embodiments guide oligonucleotides of the present invention comprise 0, 1, 2 or 3 mismatches, wherein a single mismatch may comprise several sequential nucleotides. In some embodiments guide oligonucleotides of the present invention comprise 0, 1, 2 or 3 wobble base pairs. Wobble base pairs are: G-U, I-U, I-A, and I-C base pairs.

The regular internucleosidic linkages between the nucleotides may be altered by mono- or di-thioation of the phosphodiester bonds to yield phosphorothioate esters or phosphorodithioate esters, respectively. Other modifications of the internucleosidic linkages are possible, including amidation and peptide linkers. In a preferred aspect a guide oligonucleotide of the present invention has 1, 2, 3, 4 or more phosphorothioate linkages between the most terminal nucleotides of the guide oligonucleotide (hence, preferably at both the 5′ and 3′ end), which means that in the case of 4 phosphorothioate linkages, which is a specifically preferred aspect, the ultimate 5 nucleotides are linked accordingly. It will be understood by the skilled person that the number of such linkages may vary on each end, depending on the target sequence, or based on other aspects, such as stability, toxicity and/or efficiency. In one embodiment of the invention, a guide oligonucleotide according to the present invention comprises a substitution of one of the non-bridging oxygens in the phosphodiester linkage. This modification slightly destabilizes base-pairing but adds significant resistance to nuclease degradation. The exact chemistries and formats may depend on the oligonucleotide construct and differ from application to application, and may be worked out in accordance with the wishes and preferences of those of skill in the art. It is believed in the art that four or more consecutive DNA nucleotides in an oligonucleotide create so-called ‘gapmers’ that—when annealed to their RNA cognate sequences—induce cleavage of the target RNA by RNase H. According to the present invention, RNase H cleavage of the target RNA is generally to be avoided as much as possible.

A guide oligonucleotide according to the invention is normally longer than 10 nucleotides, preferably more than 11, 12, 13, 14, 15, 16, still more preferably more than 17 nucleotides. In one embodiment the guide oligonucleotide according to the invention comprises 20 to 50 nucleotides. The oligonucleotide according to the invention is preferably shorter than 100 nucleotides, still more preferably shorter than 60 nucleotides. In a preferred aspect, the oligonucleotide according to the invention comprises 18 to 70 nucleotides, more preferably comprises 18 to 60 nucleotides, and even more preferably comprises 18 to 50 nucleotides. Hence, in a particularly preferred aspect, the oligonucleotide of the present invention comprises 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. In another preferred aspect, at either end or at both termini of a guide oligonucleotide according to the present invention inverted deoxyT or dideoxyT nucleotides may be incorporated.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of hADAR to deaminate adenosines in a non-discriminative manner, reacting with any adenosine it encounters. The specificity of hADAR1 and 2 can be increased by ensuring several mismatches in the dsRNA that presumably help to position the dsRNA binding domains in a way that has not been clearly defined yet. Additionally, the deamination reaction itself can be enhanced by providing a guide oligonucleotide that comprises a mismatch opposite the adenosine to be edited. The mismatch is preferably created by providing a targeting portion having a cytidine opposite the adenosine to be edited. As an alternative, also uridines may be used opposite the adenosine, which, understandably, will not result in a ‘mismatch’ because U and A pair. Upon deamination of the adenosine in the target strand, the target strand will obtain an inosine which, for most biochemical processes, is “read” by the cell's biochemical machinery as a G. Hence, after A to I conversion, the mismatch has been resolved, because I is perfectly capable of base pairing with the opposite C in the targeting portion of the oligonucleotide construct according to the invention. After the mismatch has been resolved due to editing, the substrate is released and the oligonucleotide construct-editing entity complex is released from the target RNA sequence, which then becomes available for downstream biochemical processes, such as splicing and translation. The desired level of specificity of editing the target RNA sequence may depend on the application. Following the instructions in the present disclosure, those of skill in the art will be capable of designing the complementary portion of the oligonucleotide according to their needs, and, with some trial and error, obtain the desired result.

In an aspect of the invention is provided a composition comprising at least one guide oligonucleotide according to the invention, preferably wherein said composition comprises at least one excipient, and/or wherein said guide oligonucleotide comprises at least one conjugated ligand, that may further aid in enhancing the targeting and/or delivery of said composition and/or said guide oligonucleotide to a tissue and/or cell and/or into a tissue and/or cell. Compositions as described here are herein referred to as a composition according to the invention. A composition according to the invention can comprise one or more guide oligonucleotides according to the invention. In the context of this invention, an excipient can be a distinct molecule, but it can also be a conjugated moiety. In the first case, an excipient can be a filler, such as starch. In the latter case, an excipient can for example be a targeting ligand that is linked to the guide oligonucleotide according to the invention.

In preferred embodiments of this aspects, such compositions can further comprise a cationic amphiphilic compound (CAC) or a cationic amphiphilic drug (CAD). A CAC is generally a lysosomotropic agent and a weak base that can buffer endosomes and lysosomes (Mae et al. J Contr Rel 2009, 134: 221). Compositions that further comprise a CAC preferably have an improved parameter for RNA editing as compared to a similar composition that does not comprise said CAC. Examples of CACs can be found in for example WO 2018/007475 or WO 2018/134310.

In a preferred embodiment, the composition according to the invention is for use as a medicament, preferably for use in the treatment of cardiovascular disease, hypercholesterolemia, liver injury, and/or alcohol-induced steatohepatitis. The composition according to the invention is then a pharmaceutical composition. A pharmaceutical composition usually comprises a pharmaceutically accepted carrier, diluent and/or excipient. In a preferred embodiment, a composition according to the invention comprises a guide oligonucleotide as defined herein and optionally further comprises a pharmaceutically acceptable formulation, filler, preservative, solubilizer, carrier, diluent, excipient, salt, adjuvant and/or solvent. Such pharmaceutically acceptable carrier, filler, preservative, solubilizer, diluent, salt, adjuvant, solvent and/or excipient may for instance be found in Remington: The Science and Practice of Pharmacy (20^{th} edition, Baltimore, MD; Lippincott, Williams & Wilkins, 2000). The guide oligonucleotide according to the invention may possess at least one ionizable group. An ionizable group may be a base or acid and may be charged or neutral. An ionizable group may be present as ion pair with an appropriate counterion that carries opposite charge(s). Examples of cationic counterions are sodium, potassium, cesium, Tris, lithium, calcium, magnesium, trialkylammonium, triethylammonium, and tetraalkylammonium. Examples of anionic counterions are chloride, bromide, iodide, lactate, mesylate, besylate, triflate, acetate, trifluoroacetate, dichloroacetate, tartrate, phosphate, and citrate.

A pharmaceutical composition according to the invention may comprise an aid in enhancing the stability, solubility, absorption, bioavailability, activity, pharmacokinetics, pharmacodynamics, cellular uptake, and/or intracellular trafficking of the guide oligonucleotide, in particular an excipient capable of forming complexes, nanoparticles, microparticles, nanotubes, nanoparticles, nanogels, virosomes, exosomes, hydrogels, poloxamers or pluronics, polymersomes, colloids, microbubbles, vesicles, micelles, lipoplexes, and/or liposomes. Examples of nanoparticles include polymeric nanoparticles, (mixed) metal nanoparticles, carbon nanoparticles, gold nanoparticles, lipid nanoparticles, magnetic nanoparticles and peptide nanoparticles, and combinations thereof. An example of the combination of nanoparticles and oligonucleotides is a spherical nucleic acid (SNA; Barnaby et al. Cancer Treat. Res. 2015, 166: 23).

A preferred composition according to the invention comprises at least one excipient that may further aid in enhancing the targeting and/or delivery of the guide oligonucleotide to the tissue and/or a cell and or into a tissue and/or a cell. A preferred tissue or cell is a liver cell.

Many of the potential excipients are known in the art and may be categorized as a first type of excipient. Examples of first type of excipients include polymers (e.g. polyethyleneimine (PEI), polypropyleneimine (PPI), dextran derivatives, butylcyanoacrylate (PBCA), hexylcyanoacrylate (PHCA), poly(lactic-co-glycolic acid) (PLGA), polyamines (e.g. spermine, spermidine, putrescine, cadaverine), chitosan, poly(amido amines) (PAMAM), poly(ester amine), polyvinyl ether, polyvinyl pyrrolidone (PVP), polyethylene glycol (PEG), cyclodextrins, hyaluronic acid, colominic acid, and derivatives thereof), dendrimers (e.g. poly (amidoamine)), lipids (e.g. 1,2-dioleoyl-3-dimethylammonium propane (DODAP), dioleoyldimethylammonium chloride (DODAC), phosphatidylcholine derivatives (e.g. 1,2-distearoyl-sn-glycero-3-phosphocholine (DSPC)), lyso-phosphatidylcholine derivatives (e.g. 1-stearoyl-2-lyso-sn-glycero-3-phosphocholine (S-LysoPC)), sphingomyelin, 2-(3-bis-(3-aminopropyl)amino) propylamino)-N-ditetradecyl carbamoyl methylacetamide (RPR209120), phosphoglycerol derivatives (e.g. 1,2-dipalmitoyl-sn-glycero-3-phosphoglycerol, sodium salt (DPPG-Na), phosphatidic acid derivatives (e.g. 1,2-distearoyl-sn-glycero-3-phosphatidic acid, sodium salt (DSPA), phosphoethanolamine derivatives (e.g. dioleoyl-L-R-phosphatidylethanolamine (DOPE), 1,2-distearoyl-sn-glycero-3-phospho ethanolamine (DSPE), 2-diphytanoyl-sn-glycero-3-phosphoethanolamine (DPhyPE)), N-(1-(2,3-dioleoyloxy)propyl-N,N,N-trimethylammonium (DOTAP), N-(1-(2,3-dioleyloxy) propyl)-N,N,N-trimethylammonium (DOTMA), 1,3-di-oleoyloxy-2-(6-carboxy-spermyl) propylamid (DOSPER), (1,2-dimyristoyloxypropyl-3-dimethylhydroxyethylammonium (DMRIE), (N1-cholesteryloxycarbonyl-3,7-diazanonane-1,9-diamine (CDAN), dimethyl dioctadecylammonium bromide (DDAB), 1-palmitoyl-2-oleoyl-sn-glycerol-3-phosphocholine (POPC), (b-L-arginyl-2,3-L-diaminopropionic acid N-palmityl-N-oleyl amide trihydrochloride (AtuFECTO1), N,N-dimethyl-3-aminopropane derivatives (e.g. 1,2-distearoyl-N,N-dimethyl-3-aminopropane (DSDMA), 1,2-dioleyloxy-N,N-dimethyl-3-aminopropane (DoDMA), 1,2-dilinoleyloxy-N,N-3-dimethylaminopropane (DLinDMA), 2,2-dilinoleyl-4-dimethylaminomethyl (1,3)-dioxolane (DLin-K-DMA), DLinKC2DMA, DLinMC3DMA (MC3), phosphatidylserine derivatives (1,2-dioleyl-sn-glycero-3-phospho-L-serine, sodium salt (DOPS)), transfection reagents, proteins (e.g. albumin, gelatins, atelocollagen), and linear or cyclic peptides (e.g. protamine, PepFects, NickFects, polyarginine, hexa-arginine, polylysine, polyornithine, CADY, MPG, cell-penetrating peptides (CPPs), cell-translocating peptides (CTPs), targeting peptides, endosomal escape peptides). Carbohydrates and carbohydrate clusters, when used as distinct compounds, are also suitable for use as a first type of excipient.

Another preferred composition according to the invention may comprise at least one excipient categorized as a second type of excipient. A second type of excipient may comprise or contain a conjugate group as described herein to enhance targeting and/or delivery into a tissue and/or cell. The conjugate group may display one or more different or identical ligands. Examples of conjugate group ligands are e.g., peptides, vitamin, aptamers, carbohydrates or mixtures of carbohydrates, proteins, small molecules, antibodies, polymers, drugs. Examples of carbohydrate conjugate group ligands are glucose, mannose, fructose, maltose, galactose, N-galactosamine (GalNAc), glucosamine, N-acetylglucosamine, glucose-6-phosphate, mannose-6-phosphate, and maltotriose. A carbohydrate can also be comprised in a carbohydrate cluster portion, such as a GalNAc cluster portion. A carbohydrate cluster portion can comprise a targeting moiety and, optionally, a conjugate linker. In some embodiments, the carbohydrate cluster comprises 1, 2, 3, 4, 5 or 6, or more GalNAc groups. As used herein, “carbohydrate cluster” means a compound having one or more carbohydrate residues attached to a scaffold or linker group (Maier et al. Bioconj Chem 2003, 14: 18) In this context, “modified carbohydrate” means any carbohydrate having one or more chemical modifications relative to naturally occurring carbohydrates. As used herein, “carbohydrate derivative” means any compound which may be synthesized using a carbohydrate as a starting material or intermediate. As used herein, “carbohydrate” means a naturally occurring carbohydrate, a modified carbohydrate, or a carbohydrate derivative. Both types of excipients may be combined into one single composition as identified herein. An example of a trivalent N-acetylglucosamine cluster is described in WO 2017/062862, which also described a cluster of sulfonamide small molecules. An example of a single conjugate of the small molecule sertraline has also been described (Ferr\tilde{e}s-Coy et al. Mol. Psych. 2016, 21: 328) as well as conjugates of protein-binding small molecules, including ibuprofen (U.S. Pat. No. 6,656,730), spermine (Noir et al. J. Am. Chem. Soc. 2008, 130: 13500), anisamide (Nakagawa J. Am. Chem. Soc. 2010, 132, 8848) and folate (Dahmen Mol. Ther. Nucl. Acids 2012, 1, e7). Examples of lipid conjugates include fatty acids and their derivatives, e.g. palmityl, palmitoyl, stearyl, stearoyl, myristyl, myristoyl, lauryl, lauroyl, arachidonyl, arachidonoyl, behenyl, behenoyl, lignoceryl, lignoceroyl, sapienyl, sapienoyl, oleyl, oleoyl, elaidyl, elaidoyl, vaccenyl, vaccenoyl, linoleyl, linoleoyl, EPA, DHA, cholesteryl, steroid, w-3 fatty acids, w-6 fatty acids, in which one or more instances of a lipid, or mixed composition of lipids, is conjugated to the oligonucleotide of the invention. Examples of lipid conjugates of oligonucleotides have been described (WO 2019/232255; Biscans J. Control. Rel. 2019, 302, 116; Biscans Nucl. Acids Res. 2019, 47, 1082; Wang Nucl. Acid Ther. 2019, 29, 245). Examples of vitamins used for conjugation are known (Winkler Ther. Deliv. 2013, 4, 791; U.S. Pat. No. 6,127,533). Conjugates of oligonucleotides with aptamers are also known in the art (Zhao Biomaterials 2015, 67, 42).

Antibodies and antibody fragments can also be conjugated to an oligonucleotide of the invention. In a preferred embodiment, an antibody or fragment thereof targeting tissues of specific interest, particularly retinal or and/or corneal tissue, is conjugated to an oligonucleotide of the invention. Examples of such antibodies and/or fragments are for example targeted to CD71 (transferrin receptor; WO 2016/179257; Sugo J. Control. Rel. 2016, 237, 1). Other oligonucleotide conjugates are known to those skilled in the art and have been reviewed by Winkler et al. (Ther. Deliv. 2013, 4, 791, Manoharan Antisense Nucl. Acid Dev 2004, 12, 103) and Ming et al. (Adv. Drug Deliv. Rev. 2015, 87, 81).

The skilled person may select, combine and/or adapt one or more of the above or other alternative excipients and delivery systems to formulate and deliver a guide oligonucleotide for use in the present invention.

Compounds that are comprised in a composition according to the invention can also be provided separately, for example to allow sequential administration of the active ingredient of the composition according to the invention. In such a case, the composition according to the invention is a combination of compounds comprising at least one guide oligonucleotide according to the invention with or without a conjugated ligand, at least one excipient, and optionally a CAC as described above.

Preferably, the pharmaceutical composition is for local liver administration, preferably by direct injection, or by systemic delivery, and is preferably dosed in an amount ranging from about 0.1 mg/kg to about 1000 mg/kg of total guide oligonucleotide per dose, more preferably between about 1 mg/kg and about 100 mg/kg, still more preferably between about 5 mg/kg and about 50 mg/kg. The present invention also relates to a pharmaceutical composition according to the invention, wherein the pharmaceutical composition is for injection and is dosed in an amount ranging from 10 to 10000 mg of total guide oligonucleotide, more preferably between 100 and 1000 mg, still more preferably between 50 and 500 mg. of total guide oligonucleotide, generally depending on dosing regimen, weight, gender, and/or age of the subject that is treated. Dosing regimens may vary from once in a lifetime (in the case of DNA editing) to several injection over the lifetime of the patients, such as weekly, monthly, quarterly, every six months or every year. Dosing may comprise a loading dose followed by maintenance doses, which do not have to be the same. Depending on clinical outcomes such dosages and dosing regimens may be adjusted. The present invention also relates to a viral vector expressing a guide oligonucleotide according to the invention. In yet another aspect, the invention relates to a guide oligonucleotide according to the invention, a pharmaceutical composition according to the invention, or a viral vector according to the invention, for use as a medicament. In yet another aspect, the invention relates to a guide oligonucleotide according to the invention, a pharmaceutical composition according to the invention, or a viral vector according to the invention, for use in the treatment, prevention, or delay of cardiovascular disease, preferably hypercholesterolemia. In yet another aspect, the invention relates to a guide oligonucleotide according to the invention, a pharmaceutical composition according to the invention, or a viral vector according to the invention, for use in the treatment, prevention, or delay of liver injury. In yet another aspect, the invention relates to a guide oligonucleotide according to the invention, a pharmaceutical composition according to the invention, or a viral vector according to the invention, for use in the treatment, prevention, or delay of alcohol-induced steatohepatitis. In yet another embodiment, the invention relates to the use of a guide oligonucleotide according to the invention, a pharmaceutical composition according to the invention, or a viral vector according to the invention, for the treatment, prevention, or delay of cardiovascular disease, preferably hypercholesterolemia.

The term “pre-mRNA” refers to a non-processed or partly processed precursor mRNA that is synthesized from a DNA template of a cell by transcription, such as in the nucleus.

Improvements in means for providing an individual or a cell, tissue, organ of said individual with a guide oligonucleotide according to the invention, are anticipated considering the progress that has already thus far been achieved. Such future improvements may of course be incorporated to achieve the mentioned effect on restructuring of mRNA using a method of the invention. A guide oligonucleotide according to the invention can be delivered as is to an individual, a cell, tissue, or organ of said individual. When administering a guide oligonucleotide according to the invention, it is preferred that the guide oligonucleotide is dissolved in a solution that is compatible with the delivery method.

The skilled person may select and adapt any of the above or other commercially available alternative excipients and delivery systems to package and deliver a guide oligonucleotide for use in the current invention to deliver it for the prevention, treatment or delay of a cardiovascular disease or condition, such as hypercholesterolemia, or for the treatment or prevention of liver injury or alcohol-induced steatohepatitis.

In all embodiments of the invention, the term ‘treatment’ is understood to also include the prevention and/or delay of the cardiovascular disease or condition. An individual, which may be treated using a guide oligonucleotide according to the invention may already have been diagnosed as having a cardiovascular related disease or condition. Alternatively, an individual which may be treated using a guide oligonucleotide according to the invention may not have yet been diagnosed as having a cardiovascular related disease or condition such as hypercholesterolemia but may be an individual having an increased risk of developing such disease or condition in the future given his or her genetic background. A preferred individual (or subject) is a human individual (or subject). In a preferred embodiment the cardiovascular related disease or condition is hypercholesterolemia, more preferably caused by limited availability of the LDL receptor on the cell membrane, more preferably on the cell membrane of hepatocytes. Accordingly, the invention further provides a guide oligonucleotide according to the invention, or a viral vector according to the invention, or a composition according to the invention for use as a medicament, for treating a cardiovascular related disease or condition requiring RNA editing of PCSK9 (pre-) mRNA and for use as a medicament for the prevention, treatment or delay of a cardiovascular related disease or condition caused by or that is related to the activity of PCSK9. Each feature of said use has earlier been defined herein.

The invention further provides the use of a guide oligonucleotide according to the invention, or of a viral vector according to the invention, or a (pharmaceutical) composition according to the invention for the treatment of a cardiovascular related disease or condition requiring RNA editing (or deamination of the specified adenosine) of PCSK9 (pre-) mRNA. In a preferred embodiment, and for all aspects of the invention, the PCSK9 related disorder, disease, or condition is caused by, or related to, the wild type or a gain-of-function mutant of the PCSK9 gene.

The invention further provides the use of a guide oligonucleotide according to the invention, or of a viral vector according to the invention, or a composition according to the invention for the preparation of a medicament, for the preparation of a medicament for treating a cardiovascular related disease or condition requiring RNA editing of a specified adenosine within the PCSK9 (pre-) mRNA and for the preparation of a medicament for the prevention, treatment or delay of cardiovascular related disease or condition, such as those caused by excess LDL in the circulation. Therefore, in a further aspect, there is provided the use of a guide oligonucleotide, viral vector or composition as defined herein for the preparation of a medicament, for the preparation of a medicament for treating a condition requiring RNA editing of a specified target adenosine of PCSK9 (pre-) mRNA and for the preparation of a medicament for the prevention, treatment or delay of a cardiovascular related disease or condition. More preferably, the autocleavage site around position Gln152 in the PCSK9 protein is disabled.

A treatment in a use or in a method according to the invention is at least once, lasts one week, one month, several months, 1, 2, 3, 4, 5, 6 years or longer, such as lifelong. It is to be understood that a particularly preferred treatment as disclosed herein does not edit the cell's DNA and the mutated (pre-) mRNA is constantly being produced by the cell and that mutated (pre-) mRNA may require continuous or intermittent editing to treat the disease. An obvious advantage of RNA editing over DNA editing is the possibility to tune the dose and dosing regimen in accordance with the severity of the disease, its time course, and the individual need of the patient. Treatment by targeting RNA, which is short-lived, can be permanently or temporarily halted to deal with potential adverse events or for reasons of drug-drug interactions should the individual need treatment with a drug that is incompatible with the editing approach. Each guide oligonucleotide or equivalent thereof as defined herein for use according to the invention may be suitable for direct administration to a cell, tissue and/or an organ in vivo of individuals already affected or at risk of developing the disease or condition, and may be administered directly in vivo, ex vivo or in vitro. The frequency of administration of a guide oligonucleotide, composition, compound, or adjunct compound of the invention may depend on the formulation (for example delayed release formulations) of said guide oligonucleotide or the route of administration. Oligonucleotides, including guide oligonucleotides, can be applied topically (to the skin, cornea of the eye, or intravitreally, or locally such by intra-tumoral or intra-cerebroventricular (ICV) injection, or systemic by injecting it into the circulation (blood or intrathecal fluid) or ingestion. Oligonucleotides may be administered to the lungs by inhalation or nebulization.

In a preferred embodiment, a viral vector, preferably an AAV vector as described earlier herein, as delivery vehicle for a molecule according to the invention, is administered in a dose ranging from 1×10⁹ to 1×10¹⁷ virus particles per injection, more preferably from 1×10¹⁰ to 1×10¹² virus particles per injection. The ranges of concentration or dose of guide oligonucleotides as given above are preferred concentrations or doses for in vivo, in vitro or ex vivo uses. The skilled person will understand that depending on the guide or the guide oligonucleotide used, the target cell to be treated, the gene target and its expression levels, the medium used and the transfection and incubation conditions, the concentration or dose of guide oligonucleotides used may vary and may need to be optimized further.

The invention further provides a method for RNA editing of PCSK9 pre-mRNA in a cell comprising contacting the cell, preferably a liver cell, with a guide oligonucleotide according to the invention, or a viral vector according to the invention, or a composition according to the invention. The features of this aspect are preferably those defined earlier herein. Contacting the cell with a guide oligonucleotide according to the invention, or a viral vector according to the invention, or a composition according to the invention may be performed by any method known by the person skilled in the art. Use of the methods for delivery of guide oligonucleotides, viral vectors and compositions described herein is included. Contacting may be directly or indirectly and may be in vivo, ex vivo or in vitro.

The invention further provides a method for the treatment of a cardiovascular related disease or condition requiring RNA editing of a specified target adenosine of PCSK9 pre-mRNA of an individual in need thereof (e.g. a patient suffering from hypercholesterolemia), said method comprising contacting a cell, preferably a liver cell, of said individual with a guide oligonucleotide according to the invention, or a viral vector according to the invention, or a composition according to the invention, to deaminate a specific adenosine within said pre-mRNA. The features of this aspect are preferably those defined earlier herein. Contacting the cell, preferably a liver cell, with a guide oligonucleotide according to the invention, or a viral vector according to the invention, or a composition according to the invention may be performed by any method known by the person skilled in the art. Use of the methods for delivery of guide oligonucleotides, viral vectors and compositions described herein is included. Contacting may be directly or indirectly and may be in vivo, ex vivo or in vitro. Unless otherwise indicated each embodiment as described herein may be combined with another embodiment as described herein.

The sequence information as provided herein should not be so narrowly construed as to require inclusion of erroneously identified bases. The skilled person can identify such erroneously identified bases and knows how to correct for such errors.

All patent and literature references cited in the present specification are hereby incorporated by reference in their entirety.

### EXAMPLES

### Example 1

Use of Phosphorothioate-Modified RNA Editing Guide Oligonucleotides for A-to-I Editing of hAPP Target RNA in an In Vitro Biochemical Editing Assay.

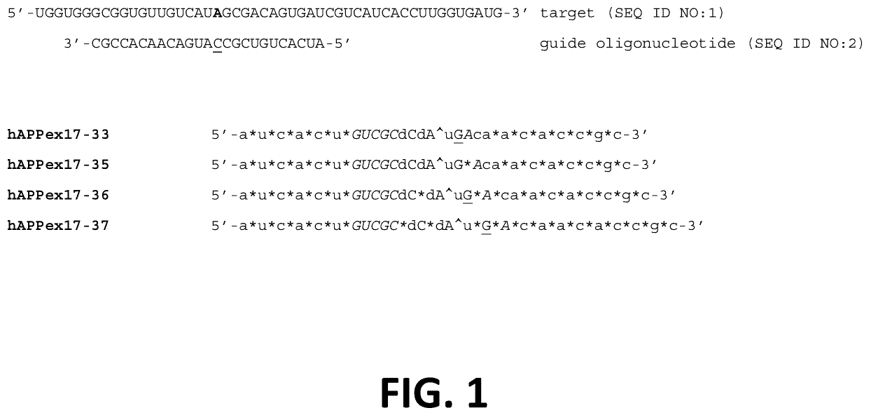

It was initially investigated whether the use of RNA editing oligonucleotides comprising phosphorothioate (PS) linkages were applicable for specific editing of human Amyloid Precursor Protein (APP) target (pre-) mRNA. To study the effect of PS linkages on hAPP target RNA editing, four guide oligonucleotides with a variety of PS linkages were designed and named hAPPex17-33, hAPPex17-35, hAPPex17-36, and hAPPex17-37, respectively (FIG. 1). The editing efficacy of all four guide oligonucleotides were measured and compared in a biochemical editing assay.

To obtain the hAPP target RNA a PCR was performed using a hAPP G-block (IDT) which contained the sequence for the T7 promotor and (a part of) the sequence of hAPP as template using forward primer 5′-CTC GAC GCA AGC CAT AAC AC-3′ (SEQ ID NO:3) and reverse primer 5′-TGG ACC GAC TGG AAA CGT AG-3′ (SEQ ID NO:4). The PCR product was then used as template for the in vitro transcription. The MEGAscript T7 transcription kit was used for this reaction. The RNA was purified on a urea gel then extracted in 50 mM Tris-Cl pH 7.4, 10 mM EDTA, 0.1% SDS, 0.3 M NaCl buffer and phenol-chloroform purified. The purified RNA was used as target in the biochemical editing assay.

Guide oligonucleotides hAPPex17-33, hAPPex17-35, hAPPex17-36, and hAPPex17-37 were annealed to the hAPP target RNA, which was done in a buffer (5 mM Tris-Cl pH 7.4, 0.5 mM EDTA and 10 mM NaCl) at the ratio 1:3 of target RNA to oligonucleotide (600 nM oligonucleotide and 200 nM target). The samples were heated at 95° C. for 3 min and then slowly cooled down to RT. Next, the editing reaction was carried out. The annealed oligonucleotide/target RNA was mixed with protease inhibitor (COMPLETE™, Mini, EDTA-free Protease I, Sigma-Aldrich), RNase inhibitor (RNasin, Promega), poly A (Qiagen), tRNA (Invitrogen) and editing reaction buffer (15 mM Tris-C pH 7.4, 1.5 mM EDTA, 3% glycerol, 60 mM KCl, 0.003% NP-40, 3 mM MgCl₂ and 0.5 mM DTT) such that their final concentration was 6 nM oligonucleotide and 2 nM target RNA. The reaction was started by adding purified ADAR2 (GenScript) to a final concentration of 6 nM into the mix and incubated for predetermined time points (0 sec, 30 sec, 1 min, 2 min, 5 min, 10 min, 25 min and 50 min) at 37° C. Each reaction was stopped by adding 95 μl of boiling 3 mM EDTA solution. A 6 μl aliquot of the stopped reaction mixture was then used as template for cDNA synthesis using MAXIMA™ reverse transcriptase kit (Thermo Fisher) with random hexamer primer (ThermoFisher Scientific). Initial denaturation of RNA was performed in the presence of the primer and dNTPs at 95° C. for 5 min, followed by slow cooling to 10° C., after which first strand synthesis was carried out according to the manufacturer's instructions in a total volume of 20 μl, using an extension temperature of 62° C. Products were amplified for pyrosequencing analysis by PCR, using the AMPLITAQ™ Gold 360 DNA Polymerase kit (Applied Biosystems) according to the manufacturer's instructions, with 1 μl of the cDNA as template. The following primers were used at a concentration of 10 μM: PYROSEQ™ Fwd hAPP, 5′-TGG GTT GAC AAA TAT CAA GAC G-3′ (SEQ ID NO:20), and PYROSEQ™ Rev hAPP Biotin, 5′-/5BiosG/CAC CAT GAT GAA TGG ATG TGT ACT-3′ (SEQ ID NO:21). PCR was performed using the following thermal cycling protocol: Initial denaturation at 95° C. for 5 min, followed by 40 cycles of 95° C. for 30 sec, 58° C. for 30 sec and 72° C. for 30 sec, and a final extension of 72° C. for 7 min.

Because inosines base-pair with cytidines during the cDNA synthesis in the reverse transcription reaction, the nucleotides incorporated in the edited positions during PCR will be guanosines. The percentage of guanosine (edited) versus adenosine (unedited) was defined by pyrosequencing. Pyrosequencing of the PCR products and data analysis were performed by the PYROMARK® Q48 Autoprep instrument (QIAGEN) following the manufacturer's instructions with 10 μl input of the PCR product and 4 μM of the following sequencing primer: hAPP-Seq, 5′-GCA ATC ATT GGA CTC AT -3′ (SEQ ID NO:22). The settings specifically defined for this target RNA strand included two sets of sequence information. The first of these defines the sequence for the instrument to analyse, in which the potential for a particular position to contain either an adenosine or a guanosine is indicated by a “/”: GGT GGG CGG TGT TGT CAT A/G GCG ACA GTG ATC GTC AT (SEQ ID NO:23). The dispensation order was defined for this analysis as follows: TGT GCG TGT GTC ACT AGC GAG CAG TG (SEQ ID NO:24). The analysis performed by the instrument provides the results for the selected nucleotide as a percentage of adenosine and guanosine detected in that position, and the extent of A-to-I editing at a chosen position will therefore be measured by the percentage of guanosine in that position.

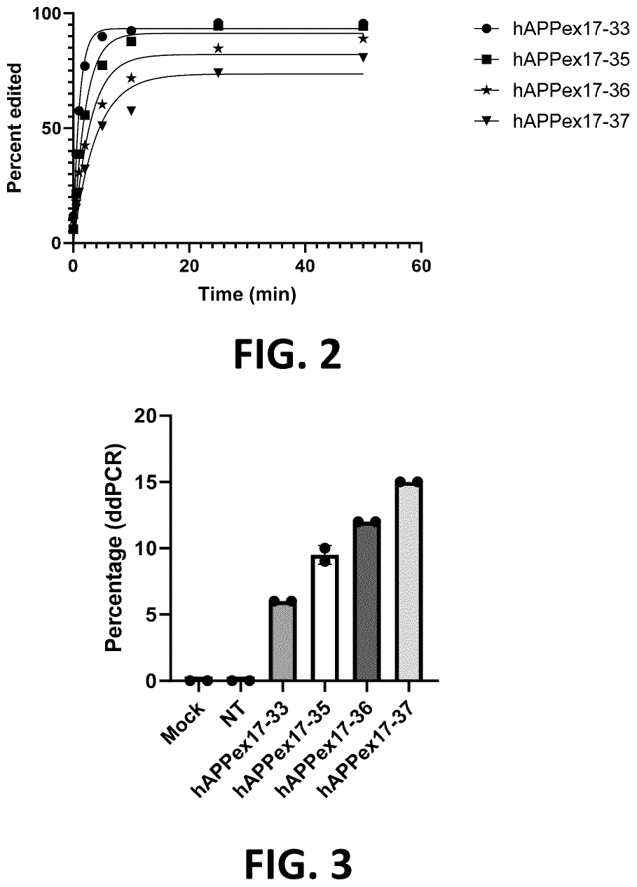

The results shown in FIG. 2 indicate that all tested guide oligonucleotides can edit the target hAPP RNA. Editing is highest with a guide oligonucleotide with no PS linkages in the proximity of the orphan base and that each addition of phosphorothioate within this region lowers the editing capability of EON, albeit not dramatically.

### Example 2

Use of Phosphorothioate-Modified RNA Editing Guide Oligonucleotides for Specific A-to-I Editing of hAPP Target RNA in Human Cells.

Next it was investigated whether the PS-modified guide oligonucleotides of the previous example would be able of edit endogenous wild-type hAPP RNA in cells. For this, human retinal pigment epithelium (RPE) cells were used. Approximately 250,000 cells per 6 well plate were seeded 24 hrs before transfection, which was performed with 100 nM guide oligonucleotide and LIPOFECTAMINE™ 2000 (Invitrogen) according to the manufacturer's instructions (at a ratio 1:2, 1 pg oligonucleotide to 2 μl LIPOFECTAMINE™ 2000). RNA was extracted from cells 48 hrs after transfection using the DIRECT-ZOL™ RNA MiniPrep (Zymo Research) kit according to the manufacturer's instructions, and cDNA was prepared using the MAXIMA™ reverse transcriptase kit (Thermo Fisher) according to the manufacturer's instructions, with a combination of random hexamer and oligo-dT primers. The cDNA was diluted 5× and 1 μL of this dilution was used as template for digital droplet PCR (ddPCR). The ddPCR assay for absolute quantification of nucleic acid target sequences was performed using BioRad's QX-200 DROPLET DIGITAL™ PCR system. 1 μl of diluted cDNA obtained from the RT cDNA synthesis reaction was used in a total mixture of 21 μl of reaction mix, including the DDPCR™ Supermix for Probes no dUTP (Bio Rad), a TAQMAN™ SNP genotype assay with the following forward and reverse primers combined with the following gene-specific probes:

Forward primer:
(SEQ ID NO: 5)
5′-CATTGGACTCATGGTGG-3′
Reverse primer:
(SEQ ID NO: 6)
5′-CAGCATCACCAAGGTG-3′
Wild type probe (HEX NFQ labeled):
(SEQ ID NO: 7)
5′-/5HEX/TGTT+GTCAT+A+G+CGACAGT/3IABkFQ/-3′
Mutant probe (FAM NFQ labeled):
(SEQ ID NO: 8)
5′-/56-FAM/TGTTGTCAT+G+GCGACAGT/3IABkFQ/-3′

A total volume of 21 μl PCR mix including cDNA was filled in the middle row of a DDPCR™ cartridge (BioRad) using a multichannel pipette. The replicates were divided by two cartridges. The bottom rows were filled with 70 μl of droplet generation oil for probes (BioRad). After the rubber gasket replacement, droplets were generated in the QX200 droplet generator. 42 μl of oil emulsion from the top row of the cartridge was transferred to a 96-wells PCR plate. The PCR plate was sealed with a tin foil for 4 sec at 170° C. using the PX1 plate sealer, followed by the following PCR program: 1 cycle of enzyme activation for 10 min at 95° C., 40 cycles denaturation for 30 sec at 95° C. and annealing/extension for 1 min at 55.8° C., 1 cycle of enzyme deactivation for 10 min at 98° C., followed by a storage at 8° C. After PCR, the plate was read and analysed with the QX200 droplet reader.

The results shown in FIG. 3 indicate that all four guide oligonucleotides cause editing. While the results from the biochemical editing assay in the previous example show that more PS linkages closer to the orphan base reduce the editing capability, in cells this appears different. This indicates that the position of the PS linkages and additional PS linkages closer to the orphan base apparently protects this region against nuclease degradation and therefore further supports their editing efficacy.

### Example 3

### Use of Phosphorothioate-Modified RNA Editing Guide Oligonucleotides for A-to-I Editing of PCSK9 Target RNA in an In Vitro Biochemical Editing Assay.

Next, it was investigated whether the use of RNA editing oligonucleotides comprising phosphorothioate (PS) linkages were also applicable for specific editing of human Proprotein convertase subtilisin/kexin type-9 (PCSK9) target (pre-) mRNA. Thirteen guide oligonucleotides with a variety of PS linkages were initially designed and named PCSK9-1 until PCSK9-13, respectively (FIG. 4). The editing efficacy of these oligonucleotides were measured and compared in a biochemical editing assay.

To obtain the PCSK9 target RNA a PCR was performed using a PCSK9 G-block (IDT) generally as described in Example 1 above. The PCR product was then used as template for the in vitro transcription. The MEGASCRIPT™ T7 transcription kit was used for this reaction. The RNA was purified on a urea gel then extracted in 50 mM Tris-Cl pH 7.4, 10 mM EDTA, 0.1% SDS, 0.3 M NaCl buffer and phenol-chloroform purified. The purified RNA was used as target in the biochemical editing assay.

Guide oligonucleotides PCSK9-1 until PCSK9-13 were annealed to the PCSK9 target RNA, which was done in a buffer (5 mM Tris-Cl pH 7.4, 0.5 mM EDTA and 10 mM NaCl) at the ratio 1:3 of target RNA to oligonucleotide (600 nM oligonucleotide and 200 nM target). The samples were heated at 95° C. for 3 min and then slowly cooled down to RT. Next, the editing reaction was carried out. The annealed oligonucleotide/target RNA was mixed with protease inhibitor (COMPLETE™, Mini, EDTA-free Protease I, Sigma-Aldrich), RNase inhibitor (RNasin, Promega), poly A (Qiagen), tRNA (Invitrogen) and editing reaction buffer (15 mM Tris-Cl pH 7.4, 1.5 mM EDTA, 3% glycerol, 60 mM KCl, 0.003% NP-40, 3 mM MgCl₂ and 0.5 mM DTT) such that their final concentration was 6 nM oligonucleotide and 2 nM target RNA. The reaction was started by adding purified ADAR2 (GenScript) to a final concentration of 6 nM into the mix and incubated for predetermined time points (0 sec, 30 sec, 1 min, 2 min, 5 min, 10 min, 25 min and 50 min) at 37° C. Each reaction was stopped by adding 95 μl of boiling 3 mM EDTA solution. A 6 μl aliquot of the stopped reaction mixture was then used as template for cDNA synthesis using MAXIMA™ reverse transcriptase kit (Thermo Fisher) with random hexamer primer (ThermoFisher Scientific). Initial denaturation of RNA was performed in the presence of the primer and dNTPs at 95° C. for 5 min, followed by slow cooling to 10° C., after which first strand synthesis was carried out according to the manufacturer's instructions in a total volume of 20 μl, using an extension temperature of 62° C. Products were amplified for pyrosequencing analysis by PCR, using the AMPLITAQ™ Gold 360 DNA Polymerase kit (Applied Biosystems) according to the manufacturer's instructions, with 2 μl of the cDNA as template. The following primers were used at a concentration of 10 μM: PYROSEQ™ Fwd PCSK9 Biotin, 5′-/5Biosg/AAG TTG CCC CAT GTC GAC TA-3′ (SEQ ID NO:17), and PYROSEQ™ Rev PCSK9, 5′-TCA CTC TGT ATG CTG GTG TCT AGG-3′ (SEQ ID NO:18). PCR was performed using the following thermal cycling protocol: Initial denaturation at 95° C. for 5 min, followed by 45 cycles of 95° C. for 30 sec, 56.1° C. for 30 sec and 72° C. for 30 sec, and a final extension of 72° C. for 7 min.

Because inosines base-pair with cytidines during the cDNA synthesis in the reverse transcription reaction, the nucleotides incorporated in the edited positions during PCR will be guanosines. The percentage of cytidines (edited) versus thymines (unedited) was defined by pyrosequencing. Pyrosequencing of the PCR products and data analysis were performed by the PYROMARK™ Q48 Autoprep instrument (QIAGEN) following the manufacturer's instructions with 10 μl input of the PCR product and 4 μM of the following sequencing primer: PCSK9-Seq, 5′-CCG TGG AGG GGT AAT-3′ (SEQ ID NO:19). The settings specifically defined for this target RNA strand included two sets of sequence information. The first of these defines the sequence for the instrument to analyze, in which the potential for a particular position to contain either a thymine or a cytidine is indicated by a “/”: CCG CTC CAG GTT CCA CGG GAT GCT CT/CG GGC AAA GAC AG (SEQ ID NO:35). The dispensation order was defined for this analysis as follows: TCG CTC AGT CAC GAT GCG TCT GCA GAC TAG (SEQ ID NO:36). The analysis performed by the instrument provides the results for the selected nucleotide as a percentage of thymine and cytidine detected in that position, and the extent of A-to-I editing at a chosen position will therefore be measured by the percentage of cytidine in that position.

The results shown in FIGS. 5A, 5B and 5C indicate that all tested guide oligonucleotides can edit the target PCSK9 RNA. Editing is highest with a guide oligonucleotide with no PS linkages in the proximity of the orphan base and that each addition of phosphorothioate within this region lowers the editing capability of EON, albeit not dramatically.

### Example 4

### RNA Editing of the Second Nucleotide of the Codon Encoding Glutamine at Position 152 of the RNA Encoding the Human PCSK9 Proprotein.

To show that the same principle as outlined above is feasible for the deamination of the second nucleotide in the codon of Gln152 in the pre-mRNA or mRNA encoding the (wild type) human PCSK9 proprotein, the inventors investigate this in similar biochemical and cellular assays as described above. For this, the guide oligonucleotides as depicted in FIG. 4 are tested, and both human hepatocytes and cynomolgus monkey hepatocytes (Thermo Fisher Scientific, cat #MKCP10) are used. Cells are seeded 24 hrs before transfection, which is performed with 100 nM guide oligonucleotide and LIPOFECTAMINE™ 2000 (Invitrogen) according to the manufacturer's instructions (at a ratio 1:2, 1 pg oligonucleotide to 2 μl LIPOFECTAMINE™ 2000). RNA is extracted from cells 48 hrs after transfection using the DIRECT-ZOL™ RNA MiniPrep (Zymo Research) kit according to the manufacturer's instructions, and cDNA is prepared using the MAXIMA™ reverse transcriptase kit (Thermo Fisher) according to the manufacturer's instructions, with a combination of random hexamer and oligo-dT primers. The cDNA is diluted 5× and 1 μL of this dilution is used as template for digital droplet PCR (DDPCR™). The DDPCR™ assay for absolute quantification of nucleic acid target sequences is performed using BioRad's QX-200 Droplet Digital PCR system. 1 μl of diluted cDNA obtained from the RT cDNA synthesis reaction is used in a total mixture of 21 μl of reaction mix, including the DDPCR™ Supermix for Probes no dUTP (Bio Rad), a Taqman SNP genotype assay with forward and reverse primers generally as described above. DDPCR™ is also performed generally as described above. RNA editing of the specific nucleotide in the Gln152 codon is likely observed.

As a next step, the inventors are investigating whether auto-cleavage is inhibited or reduced, and in a subsequent experiment, the effect of reduced PCSK9 activity is assessed in vivo, in monkeys that have a conserved sequence of the P1 auto-cleavage site in their PCSK9 gene in comparison to humans, which will then serve as a perfect animal model for use of guide oligonucleotides to edit the RNA of PCSK9 in the treatment of hypercholesterolemia.

### Example 5

Use of GalNac-Modified RNA Editing Guide Oligonucleotides for A-to-I Editing of mlDUA Target RNA in an In Vitro Biochemical Editing Assay.

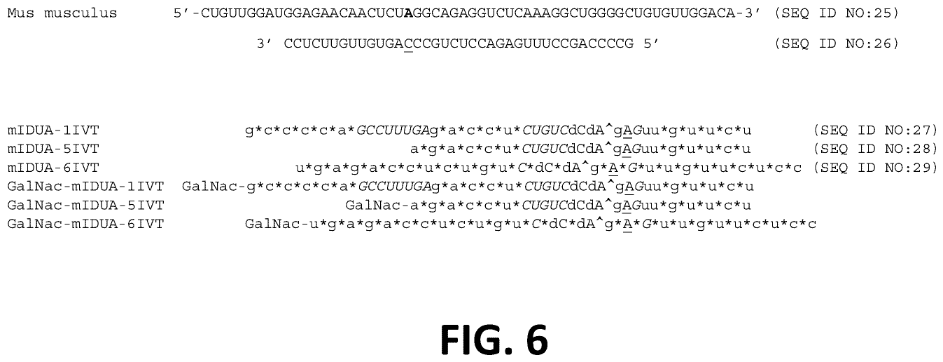

As indicated in the accompanying description, it is well known in the art that GalNAc interacts with the asialoglycoprotein receptor, which is abundantly present on hepatocytes. The inventors wondered, even though such would allow better uptake of the guide oligonucleotides by the cells, whether the conjugation of a GalNac moiety to a guide oligonucleotide would hamper the RNA editing activity of the oligonucleotide. To test this, a similar biochemical assay as described in example 1 was set up with guide oligonucleotides with and without the attachment of the GalNac moiety. One potential disease target for RNA editing is mucopolysaccharidosis type I-Hurler (MPS I-H; Hurler syndrome). This disease is caused by a c.1205G>A (W402X) mutation in the IDUA gene, which encodes the lysosomal enzyme α-L-iduronidase. Editing the A to I would potentially reverse the mutation to a wild-type sequence. A mouse model with a similar mutation (W392X) exists, with the mutation in the endogenous gene. Part of the mouse Idua sequence is provided in FIG. 6, with the mutation in bold.

The sequences of the tested guide oligonucleotides are also depicted in FIG. 6, showing that mlDUA-1IVT has the same sequence as GalNac-mlDUA-1 VT, except that the latter has a GalNac moiety attached to the 5′ terminus. The same holds true for mlDUA-5IVT/GalNac-mlDUA-5IVT and mlDUA-6IVT/GalNac-mlDUA-6IVT respectively. Means of manufacturing guide oligonucleotide-GalNAc conjugates are routine for persons of skill in the art. The editing efficacy of all six guide oligonucleotides were measured and compared in a biochemical editing assay, like what has been described in examples 1 and 3.

To obtain the mldua target RNA a PCR was performed using a mldua G-block (IDT) generally as described in Example 1 above. Transcription and purification were performed as described above.

The guide oligonucleotides were annealed to the target RNA and treated as described above. The editing reaction was performed by using purified ADAR2 as described above. Products were amplified for pyrosequencing analysis using the following primers: PYROSEQ™ Fwd mlDUA: 5′-AGT ACT CAC AGT CAT GGG GCT CA-3′ (SEQ ID NO:30), and PYROSEQ™ Rev mlDUA Biotin, 5′-/5BiosG/GCCA GGA CAC CCA CTG TAT GAT-3′ (SEQ ID NO:31). The sequence primer used was the mlDUA-Seq: 5′-TGG GGC TCA TGG CCC T-3′ (SEQ ID NO:32) and the sequence was defined by the order: GG ATG GAG AAC AAC TCT A/G GGC AGA GGT CTC AAA GG (SEQ ID NO:33) in which the potential presence of the adenosine or guanosine is indicated by the “/” and the dispensation order was defined as CGT GAT GAG ACA CTC GTA GCA GAG TCT GCA GAG CTG CA (SEQ ID NO:34).

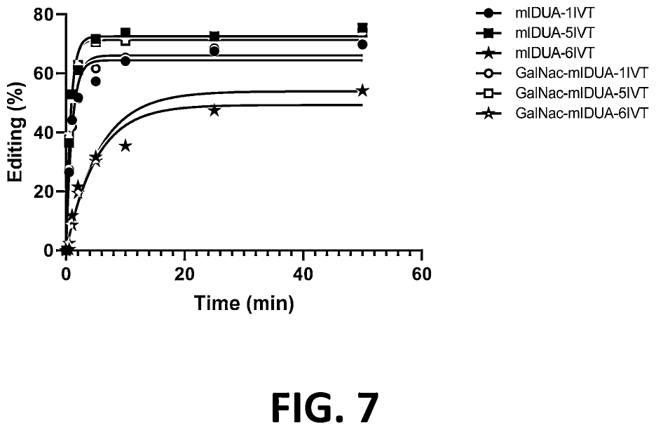

The results shown in FIG. 7 clearly indicate that all tested guide oligonucleotides can edit the target mldua RNA target very efficiently. Importantly, there appears to be no decrease in efficiency for the guide oligonucleotides that are conjugated to the GalNac moiety in comparison to the guide oligonucleotides that do not carry GalNac. Even more strikingly, the presence of the GalNac moiety did not hamper the RNA editing efficiency in any way, since the guide oligonucleotides with the same sequence and chemical modifications other than GalNac almost performed one on one. This is a strong indication that the GalNac modification (which adds in the delivery to hepatocytes) can be used in the guide oligonucleotides of the present invention and in the use thereof in the treatment of hypercholesterolemia in which hepatocytes are targeted with guide oligonucleotides to edit the PCSK9 (pre-) mRNA.

### Example 6

### Transfection in HeLa Cells of RNA Editing Guide Oligonucleotides for A-to-I Editing of PCSK9 Target RNA.

Further to the results obtained in the biochemical assay as described above, a larger set of oligonucleotides was tested for A to I editing of a wild type PCSK9 target RNA in human cervix carcinoma cells (HeLa). FIG. 4 shows part of the (pre)mRNA target sequence of the human PCSK9 transcription product as well as the corresponding part of the monkey (M. mulatta) PCSK9 transcription product. The differences between the sequences are highlighted by shading. A wide set of oligonucleotides were designed in which some are 100% complementary (except for the orphan nucleotide, and the presence of an I opposite a C) towards the human sequence and some are 100% complementary (except for the orphan nucleotide, and the presence of an I opposite a C) towards the monkey sequence. PCSK9-11, -12, -13, 17, -18, -19, -23, -24, -25, -31, -32, -33, -34, -35, -36, and -37 were tested for producing RNA editing in the HeLa cells after transfection, using a scrambled oligonucleotide and mock transfection as negative controls.

Cell culture: Human HeLa cells were cultured in DMEM/10% FBS and Human HepG2 hepatocellular carcinoma cells were cultured in MEM/10% FBS. Cells were kept at 37° C. in a 5% CO₂ atmosphere. Primary human hepatocytes were provided by Primacyt and cultured in serum-free Human Hepatocyte Maintenance Medium (Primacyt) and kept at 37° C. in a 10% CO₂ atmosphere.

Transfection of HeLa cells: A total of 0.5×10⁵ HeLa cells were transfected using LIPOFECTAMINE™ 2000 (Invitrogen) according to the manufacturer's instructions. In brief; oligonucleotides were diluted in OPTIMEM™ (Thermo Fisher) to a final concentration of 100 nM and mixed by vortexing 1:1 with a mixture of LIPOFECTAMINE™ 2000 in OPTIMEM™ in a 1:4.4 ratio (oligonucleotide:transfection agent). After incubation at RT for 20 min, mixtures were added dropwise to the wells containing fresh medium. After 24 hrs, the inoculate was aspirated and fresh culture medium was added to the wells.

48 hrs post initial exposure to the oligonucleotides, cells were collected, and total RNA was isolated from the transfected cells using the DIRECT-ZOL™ RNA Microprep kit (Zymo Research). After removal of the culture medium, cells were washed once with PBS. After complete aspiration of the PBS, 100 μL TRIREAGENT™ (Zymo Research) was added to lyse the cells and collect the intracellular material. After addition of 100 μL ethanol, the mixtures were loaded in a column and subjected to several wash steps and DNaseI treatment. After elution in a total volume of 15 μL DNase/RNase-free water, the RNA yield was determined using spectrophotometric analysis (NanoDrop) and stored at −80° C.

MAXIMA™ reverse transcriptase (RT, Thermo Fisher) was used to generate complementary DNA (cDNA). Typically, 200 ng total RNA was used in reaction mixture containing 4 μL 5×RT buffer, 1 μL dNTP mix (10 mM each), 0.5 μL Oligo(dT), 0.5 μL random hexamer (all Thermo Fisher) supplemented with DNase and RNase free water to a total volume of 20 μL. Samples were loaded in a T100 thermocycler (Bio-Rad) and initially incubated at 10 min at 25° C., followed by a cDNA reaction temperature of 30 min at 50° C. and a termination step of 5 min at 85° C. Samples were cooled down to 4° C. prior storing at −20° C.

To determine the editing efficiency, cDNA samples were used in two multiplex droplet digital PCR (DDPCR™) assays. The first DDPCR™ is designed to distinguish between cDNA species containing the original adenosine or the edited inosine, which is converted into a guanidine during cDNA synthesis. The second multiplex ddPCR quantifies the amount of PCSK9 specific cDNA molecules in the mixture using a primer/probe set targeting exons 1 and 4 or exons 9 and 10. The primer and probe sequences are listed in the table below, the cycling conditions in Table 2.

TABLE 2
Primer and probe names and sequences (+ refers to a LNA nucleotide at the 3′ side)

| Name | Sequence 5′ to 3′ |
|---|---|
| PCSK9 ex2-3 Fw | TGCTGGAGCTGGCCTTGAAGTTG (SEQ ID NO: 43) |
| PCSK9 ex3 Rv | CTGGTATTCATCCGCCCGGTAC (SEQ ID NO: 44) |
| PCSK9 ex1 Fw | TTCCGAGGAGGACGGC (SEQ ID NO: 45) |
| PCSK9 ex4 Rv | GTCTAGGAGATACACCTCCACC (SEQ ID NO: 46) |
| PCSK9 ex9 Fw | AGTTTCTCCAGGAGTGGGAAG (SEQ ID NO: 47) |
| PCSK9 ex10 Rv | AGCACCTGGCAATGGCGTAG (SEQ ID NO: 48) |
| PCSK9 A probe | /5HEX/CTTTGCC+C+A+GAGCATCC/3IABkFQ/ (SEQ ID NO: 49) |
| PCSK9 G probe | /56-FAM/CTTTGCC+C+G+GAGCATCC/3IABkFQ/ (SEQ ID NO: 50) |
| PCSK9 ex9-10 probe | /5HEX/AGCGCATGGAGGCCCAAG/3IABkFQ/ (SEQ ID NO: 51) |
| PCSK9 ex3 probe | /56-FAM/CTGCTGGAGCTGACGGAGGC/3IABkFQ/ (SEQ ID NO: 52) |

The cycling conditions were as follows: enzyme activation at 95° C. for 10 min, then 40 cycles of denaturation at 95° C. for 30 sec, annealing/extension at 64° C. for 1 min, followed by enzyme deactivation at 98° C. for 10 min, after which the product was stored at 4° C.

In total, 0.5 μL of the cDNA mix was used in a ddPCR mixture containing 10.5 μL 2×DDPCR™ Supermix for probes (Bio-Rad), 1.3 μL of primers and probes (10 μM stock concentration), supplemented with 4.8 μL DNase and RNase free water in a total volume of 21 μL. Droplets were generated using the droplet generator (Bio-Rad) with the aid of 70 μL droplet generation oil (Bio-Rad). The resulting 40 μL of droplets were transferred to a 96-well plate (Eppendorf), sealed with a tin foil using a PX1 plate sealer (Bio-Rad) before loading the plate into a T100 thermal cycler. After the cycling steps, the plate was transferred to a QX200 droplet reader set to measure the Fam and Hex fluorophores. Data was analyzed using the QUANTASOFT™ Analysis Pro software (Bio-Rad). Percentage of A-to-I editing was determined by dividing the number of G-containing molecules by the total (G- plus A-containing species) multiplied by 100.

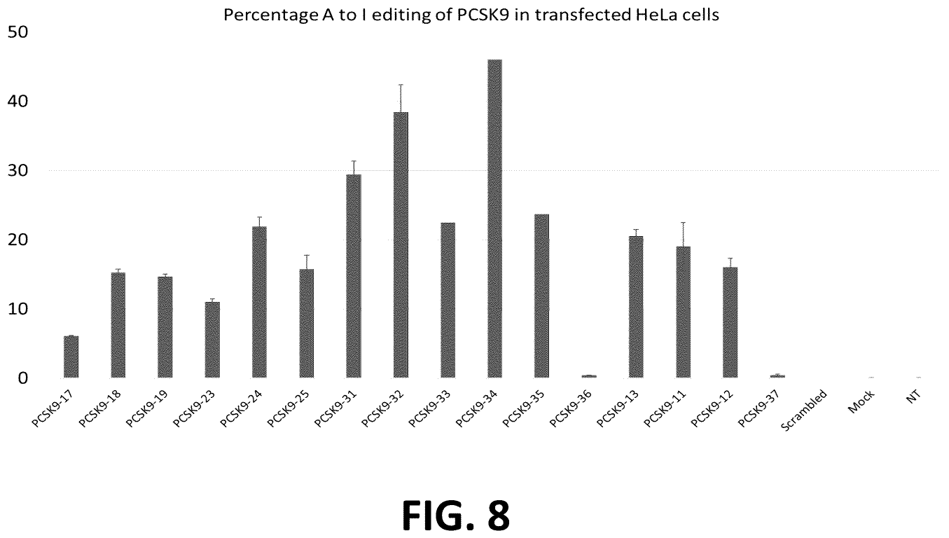

The results after transfection in HeLa cells is given in FIG. 8. Oligonucleotides PCSK9-32 and PCSK9-34 performed best in these human cells. These were designed to be 100% complementary to the monkey sequence. As shown earlier in for instance WO2017/220751 this may be due to the higher efficiency in which ADAR can bind to the dsRNA target when there are certain mismatches between the target strand and the strand that forms the double-stranded complex. From FIG. 8 it also becomes clear that oligonucleotides that comprise a large consecutive stretch of 2′-F modified nucleotides in the 5′ region of the oligonucleotide (see PCSK9-36 and PCSK9-37) hardly provided any RNA editing in comparison to the oligonucleotides that did not.

### Example 7

### Gymnotic Uptake in Human Hepatocytes of RNA Editing Guide Oligonucleotides for A-to-I Editing of PCSK9 Target RNA.

In an experiment like the previous example, the oligonucleotides given in FIG. 4 and that were tested in human HeLa cells, were then used for gymnotic uptake (=no transfection, just exposure) in human hepatocytes to determine whether the oligonucleotides could produce RNA editing of an endogenous PCSK9 target RNA in liver cells. For the gymnotic treatment of the human primary hepatocytes, 1.0×10⁵ cells were seeded in a collage-coated 96-well plate. The following day, 100 μL of fresh medium containing 1, 3 or 10 μM of each oligonucleotide was added to the cells. After 48 hrs incubation, the medium was aspirated, and total RNA was isolated. Determination of RNA editing was performed generally as described in Example 6.

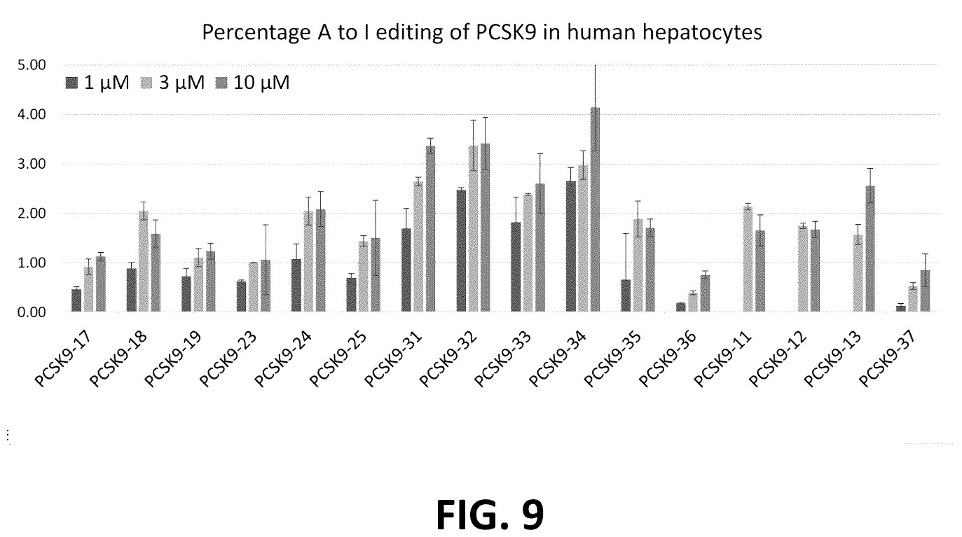

The results of this gymnotic uptake experiment are shown in FIG. 9, again indicating that oligonucleotides PCSK9-32 and PCSK9-34 performed best and that in general a dose-dependent increase of editing could be observed. Like the results obtained in HeLa cells, PCSK9-36 and PCSK-37 hardly showed any RNA editing.

### Example 8

### GalNAc-Dependent Uptake in a Human Hepatocyte Cell Line (HepG2) and Subsequent A-to-I Editing of Actin B Target RNA.

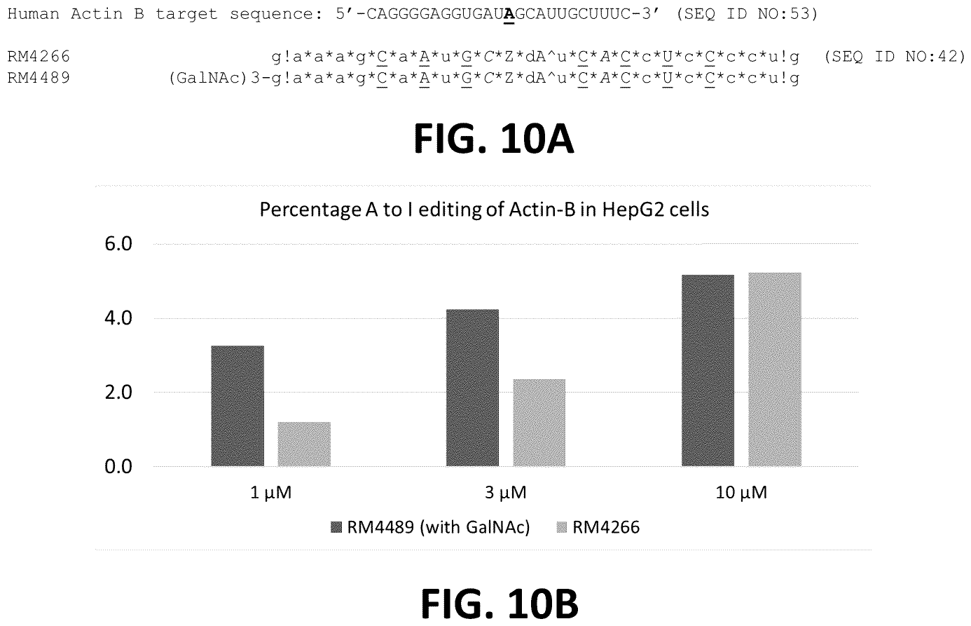

To determine the role of a GalNAc moiety and the ability for EONs attached to GalNAc to enter liver cells more efficiently, a subsequent experiment was performed in which two oligonucleotides (RM4266 minus GalNAc; and RM4489 with GalNAc attached to the 5′ terminus) were tested for their ability to produce RNA editing of an Actin B target RNA. The 5′ attached GalNAc (like all oligonucleotides that have this at the 5′ as shown in FIG. 4) was product #ON-133 (Hongene Biotech). Sequences and chemical modifications of the ActB oligonucleotides are given in FIG. 10A. The two oligonucleotides were tested in three concentrations in human hepatocyte HepG2 cells after gymnotic uptake. For the treatment of HepG2 using the two EONs, 1.25×10⁴ cells were seeded in wells of a 96-well plate. The following day, 100 μL of fresh medium containing 1, 3 or 10 μM EON was added to the cells. After 48 hrs incubation, the medium was aspirated, and total RNA was isolated. Determination of RNA editing was performed generally as described in Example 6.

The results of this experiment are shown in FIG. 10B. In the range of 1 and 3 μM a clear dose-dependent effect could be observed in which the GalNAc-associated oligonucleotide (RM4489) outperformed the non-conjugated oligonucleotide (RM4266) indicating that attachment of GalNAc to the 5′ terminus of the oligonucleotide has a beneficial effect on cell entry, trafficking and/or RNA editing efficiency.

### Example 9

Effect on Endogenous PCSK9 Protein after Editing the PCSK9 Target RNA in Human Cells.

A similar transfection experiment using human HeLa cells and an endogenous human PCSK9 transcript editing experiment was executed, similar as what has been described in example 6 above, but now for the purpose of addressing the effect of the transcript editing on the resulting PCSK9 protein translated from such transcripts.

It has been demonstrated that the substitution of the Glutamine (Q) at position 152 in the PCSK9 protein to an Arginine (R) abolishes the autocleavage site, rendering the zymogen Pro-PCSK9 inactive (Benjannet et al. 2012). This amino acid substitution can be mediated by the EON-directed ADAR deaminase of the adenosine at nucleotide position 745, as outlined in detail herein. In this example, HeLa cells were exposed 48 hours to the listed EONs after which the intracellular protein was collected. After correction of the total protein concentration, samples were loaded on a 4-10% denaturing SDS-PAGE gel and subjected to electrophoreses to separate the proteins. After transfer to a nitrocellulose membrane, the PCSK9 and Pro-PCSK9 proteins were visualized using PCSK9-specific antibodies (Rabbit monoclonal #85813 Cell Signaling Technology) followed by secondary antibodies (IRDye 680RD Goat anti-Rabbit). Beta-tubulin served as a loading control with the aid of mouse monoclonal antibodies (Santa Cruz sc-166729 and IRDye 800CW Goat anti-Mouse). Simultaneously, total RNA derived from HeLa cells treated under similar conditions was isolated as described previously and subjected to quantitative ddPCR assays to determine the percentage of A-to-I editing of PCSK9.

HeLa cells were seeded at a density of 5×10⁴ cells per well of a 24-wells plate and transfected the following day using Lipofectamine 2000 and 100 nM human EON per well. EONs were RM4356 (PCSK9-17), RM4357 (PCSK9-18), RM4358 (PCSK9-19), RM4362 (PCSK9-23), RM4363 (PCSK9-24), and RM4364 (PCSK9-25), see FIG. 4. A mock transfection was taken along as a negative control. After 48 hrs protein was collected by lysing the cells with standard RIPA buffer and total RNA was isolated using the Zymogene RNA isolation protocol. Total protein concentration was determined using a standardized BCA assay.

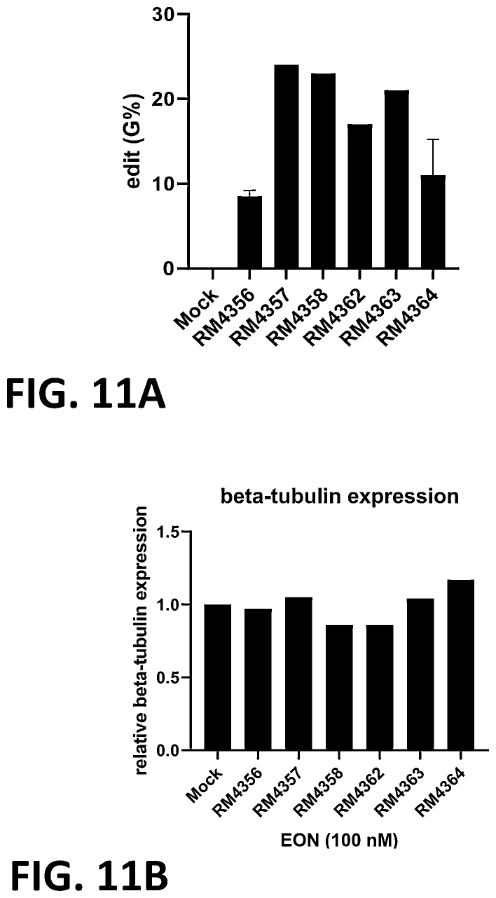

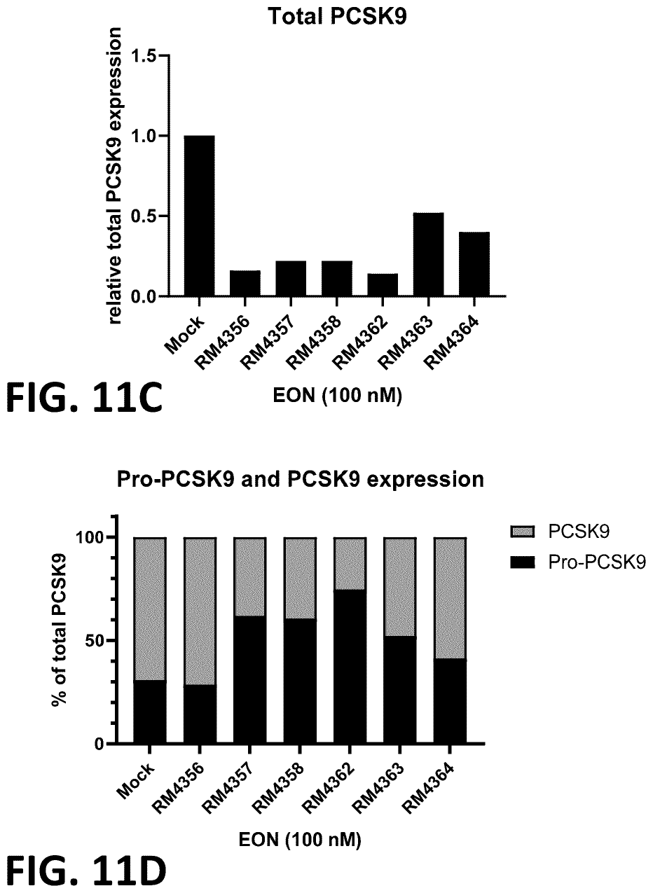

First, the percentage editing was determined by ddPCR as described in example 6. FIG. 11A shows the results in which a similar pattern of editing is observed as seen in FIG. 8 with these EONs, although in this later experiment EON PCSK9-18 outperformed EON PCSK9-24.

The corrected amount of protein was loaded on a 4-10% SDS-PAGE gel. Electrophoreses was carried out, followed by transfer of the separated proteins onto a nitrocellulose membrane and successive immunoblotting. The resulting bands were visualized using a Bio-Rad GEL DOC™ Gel Imaging system and quantified with the ImageJ software. Results for the beta-tubulin expression is shown in FIG. 11B, indicating relatively equal amounts of protein present in the seven samples.

On the western blot, the cleaved PCSK9 protein and the un-cleaved PCSK9 protein (also referred to as Pro-PCSK9) can be readily distinguished using the same anti-human PCSK9 antibody and amounts can be calculated based on these. FIG. 11C shows the total PCSK9 protein amount found in the seven samples, clearly indicating a significant decrease in PCSK9 protein abundance after EON treatment, wherein the amount in the mock-treated sample was set as 1.0 and FIG. 11D shows the ratio between the amount of processed PCSK9 (PCSK9) and un-cleaved PCSK9 (Pro-PCSK9) together contributing to the total amount of PCSK9 protein found in these treated HeLa cells.

The resulting percentage PCSK9 RNA editing is measured up to 23.8%, whereas the effect on the total level of PCSK9 protein is much greater (>80%). It is postulated here that the relationship between these values rests in the dominant negative effect of the un-cleaved zymogen as proposed by Benjannet et al. (2012). In an unedited (e.g., wild-type) situation, during translation, the pro-protein is cleaved after the Q152 amino-acid and chaperones the active PCSK9 protein via the trans-Golgi network to be excreted in the extracellular matrix. However, if the zymogen is not cleaved, the remaining protein remains retained in the endoplasmic reticulum (ER) leading to increased degradation of the protein. In addition, Benjannet et al. (2012) also demonstrated, albeit with different Q152 mutants, that co-expression of the wild-type protein next to the mutated versions, resulted in a reduction in the total PCSK9 protein by means of unsolicited oligomerization between the two species. As this is shown for mutants displaying similar characteristics to the Q152R substitution, it seems that this phenomenon will take place in cells exposed to the listed EONs, explaining the disbalance between the percentage of RNA editing and total protein isolated from the exposed cells.

Lastly, the effect of the editing on the RNA can also be appreciated by defining the ratio between the un-cleaved Pro-PCSK9 zymogen and the active PCSK9 protein. In untreated cells, the ratio has a 70%-30% balance, whereby 30% un-cleaved pro-PCSK9 zymogen and 70% cleaved PCSK9 protein is measured, which is not affected by the treatment itself (Mock). After exposure to the editing EONs, this ratio shifts to a 25%-25% in favor of the un-cleaved Pro-PCSK9 zymogen. This follows the same hypothesis mentioned above whereby the RNA edited uncleavable Pro-PCSK9 zymogen disrupt the normal processing of the PCSK9 protein in an early stage of translation. Importantly, the internal control protein beta-tubulin is not affected by the treatment.

### Example 10

A-to-I Editing in Monkey PCSK9 Target RNA after Transfection of AONs in Non-Human Primate (NHP) Primary Hepatocytes.

Like the experiment as shown in example 7 above, oligonucleotides PCSK9-17, -18, -19, -23, -24, -25, -31, -32, -33, -34, -35, and -36 were used in an editing experiment of endogenous monkey PCSK9 transcripts in primary hepatocytes from non-human primates (NHP). The oligonucleotides were used in comparison to several negative controls (scrambled non-targeting oligonucleotide, mock, non-treated cells, no reverse transcriptase, and water).

Non-human primate-derived hepatocytes were seeded in wells of a 24-well plate at a density of 4.0×10⁵ cells per well. After overnight incubation, mixtures of 100 nM of the EONs were prepared together with LIPOFECTAMINE™ RNAiMAX transfection reagent (Thermo Fisher Scientific) at a ratio 1:2 and added to the cells in a dropwise manner. The inoculates were removed after 6 hrs and fresh culture medium was added to the wells. After a 48-hr incubation period, the cells were collected and total RNA was isolated and processed as described previously. Five nanograms of cDNA was used for the quantitative DDPCR™ assay designed to determine the rate of ADAR-mediated editing of the target adenosine. Primers and probes are listed below, the used PCR program is like the human specific PCSK9 DDPCR™. The percentage of editing was calculated by dividing the number of G-containing copies per sample by the total number of PCKS9 molecules, multiplied by 100.

| Name | Sequence 5′-3′ |
|---|---|
| Fw primer | TGCTGGAGCTGGCCCTGAAGTTG (SEQ ID NO: 53) |
| Rv primer | CTGGTATTCATCCGCCCGGTAC (SEQ ID NO: 44) |
| Target A probe | /5HEX/CTTCGCCCAGAGCATCC/3IABkFQ/ (SEQ ID NO: 54) |
| Target G probe | /56-FAM/CTTCGCCCGGAGCATC/3IABkFQ/ (SEQ ID NO: 55) |

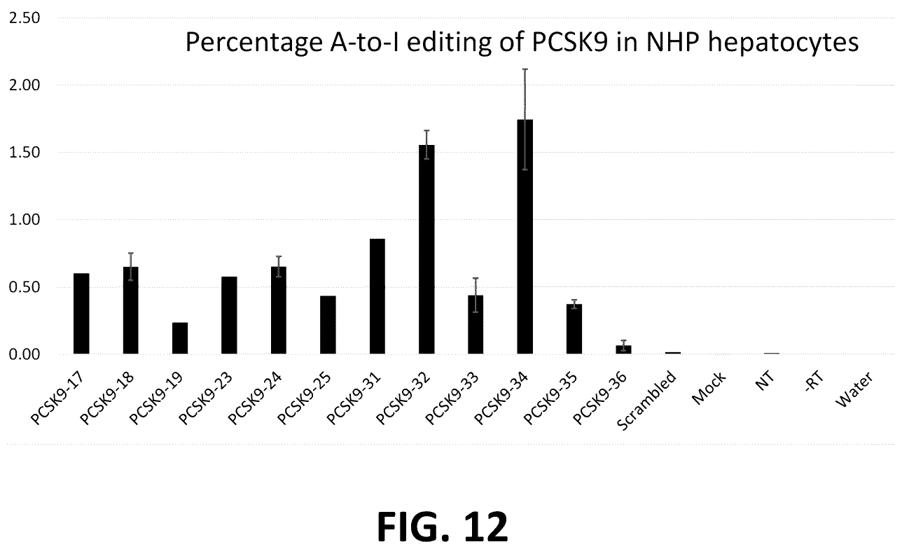

The results are shown in FIG. 12. Even though the editing percentages are relatively low, mainly because hepatocytes are notoriously hard to handle and culture/transfect/transform in vitro, the overall editing results resemble what was observed earlier in human hepatocytes (FIG. 9), clearly indicating that the oligonucleotides used herein can edit the target adenosine, also in monkey PCSK9 transcript molecules.

### Example 11

A-to-I Editing in Mouse PCSK9 Target RNA after Transfection of AONs in Mouse Primary Hepatocytes.

Like the experiment as shown in example 10 above, the following six guide oligonucleotides were used in an editing experiment using endogenous mouse PCSK9 transcripts in primary mouse hepatocytes:
PCSK9-32 (RM4416);
PCSK9-34 (RM4418);
PCSK9-35 (RM4419);
PCSK9-48 (RM4667);
PCSK9-49 (RM4668); and
PCSK9-50 (RM4669).

It was investigated whether the addition of saponin AG1856 (also referred to as a triterpene glycoside; see WO2021/122998) would boost the editing efficiency in hepatocytes.

Wild-type C57BL/6 mouse-derived hepatocytes were seeded in wells of a 24-well plate at a density of 6.0×10⁴ cells per well. After overnight incubation, medium was refreshed and mixtures of 1.0 μM of the guide oligonucleotides were prepared and added to the cells in a dropwise manner. The experiment was such that half of the hepatocytes were separately treated with guide oligonucleotide alone, and half of the hepatocytes were separately treated with AG1856 and guide oligonucleotides. 1.0 μM AG1856 was added to the wells immediately thereafter. Non-treated cells (of which half did receive AG1856) were taken as negative controls. After a 72-hr incubation period at 37° C., 10% CO₂, the cells were collected, and total RNA was isolated and processed as described above. 15 ng of cDNA was used for the quantitative digital PCR assay designed to determine the rate of ADAR-mediated editing of the target adenosine. Primers and probes are listed below, the used PCR program was like the human specific PCSK9 digital PCR. The percentage of editing was calculated by dividing the number of G-containing copies per sample by the total number of PCKS9 molecules, multiplied by 100.

| Name | Sequence 5′-3′ |
|---|---|
| mmPCSK9 ex2-3 FW | GGCCTGGCCCTGAAGTTG (SEQ ID NO: 56) |
| mmPCSK9 ex3 REV | CATGCTGGGATAATTCGCTCCA (SEQ ID NO: 57) |
| mmPCSK9 G probe | /56-FAM/CTT CGC C+C+G +GAG CAT C/3IABkFQ/ (SEQ ID NO: 58) |
| mmPCSK9 A probe | /5HEX/CTT CGC C+C+A +GAG CAT CC/3IABkFQ/ (SEQ ID NO: 59) |

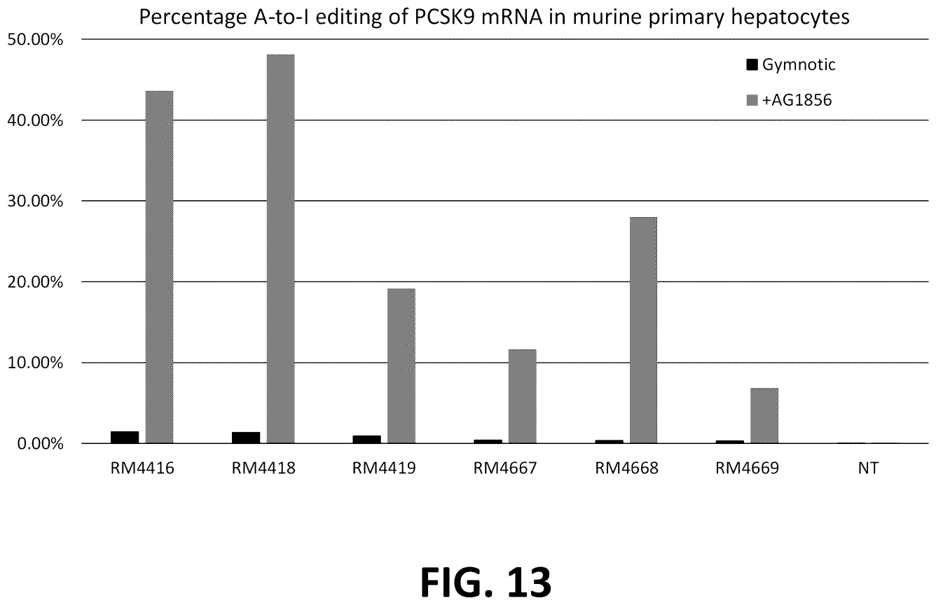

The results are given in FIG. 13 and show that the editing percentages using the gymnotic uptake of the guide oligonucleotides were relatively low, but that this could be boosted dramatically when the saponin AG1856 was added to the cell/oligonucleotide incubation, reaching editing levels of almost 50% in the case of RM4418 (guide oligonucleotide PCSK9-34; see FIG. 4). Some cells can be readily treated with oligonucleotides without the aid of transfection means, but mouse hepatocytes are difficult to use when the guide oligonucleotide should go into the cell on its own (=gymnotic uptake). This difficulty for this particular type of cell can be overcome by using an aid, such as a saponin, and more in particular (as shown here) the triterpene glycoside AG1856. This effect may also be observed in vivo, in which the saponin may be co-administered, or administered at the same time but at another location of the body or elsewhere subcutaneously, or administered before or after administration of the guide oligonucleotide, depending on the treated subject, amounts, etc. which can be easily determined by the person skilled in the art.

### Example 12

### A-to-I Editing in Mouse PCSK9 Target RNA in an In Vivo Experiment Using Mice and a Variety of Guide Oligonucleotides.

To determine the percentage of EON-mediated PCSK9 mRNA editing in vivo, an experiment is designed in which wild-type C57BL/6 mice are randomly divided over 8 groups containing 4 to 8 animals per group (for details of the groups, treatment protocol and size, see the table below). Plasma samples of all animals were collected prior to the first dose of the compounds listed to set the pre-dosage levels of PCSK9 protein and LDL-C in the blood. The number of subcutaneous doses are given in the table below, and the saponin AG1856 (in group 2, 4, and 8) is administered at the same time of the last guide oligonucleotide dosing, at another location of the mouse body. Group 1 and 2 are negative control groups. 72-hrs after the last injection, animals are sacrificed, plasma and several selected organs are collected for the analysis of PCSK9 mRNA editing percentages, liver PCSK9 protein levels and plasma PCSK9+LDL-C concentrations. It is envisioned that editing will take place in the liver of the mice in the groups receiving the PCSK9-specific guide oligonucleotides and that AG1856 further boosts the efficiency of the editing because it is envisioned that the efficiency of cell entry and/or endosomal release will be increased by the AG1856 addition. It is further envisioned that LDL-C plasma levels drop in the mice receiving a PCSK9-specific guide oligonucleotide because of the impaired PCSK9 autocleavage after RNA editing and that such levels are further lowered due to the AG1856 co-administration at the last injection.

| Group | | Dose level | No. of SC | Group | |
|---|---|---|---|---|---|
| No. | Test item | (mg/mL) | doses | size | Necropsy |
| 1 | Vehicle (PBS) | 0 | 5 | 6 | 8 |
| 2 | Vehicle + AG1856 | 0 + 3.6 | 5 (1× AG1856) | 6 | 8 |
| 3 | RM4667 | 20 | 5 | 6 | 8 |
| 4 | RM4667 + AG1856 | 20 + 3.6 | 5 (1× AG1856) | 6 | 8 |
| 5 | RM4667 | 100 | 1 | 4 + 4 | 4 & 8 |
| 6 | RM4667 + AG1856 | 100 + 3.6 | 1 | 4 + 4 | 4 & 8 |
| 7 | RM4418 | 20 | 5 | 4 | 8 |
| 8 | RM4418 + AG1856 | 20 + 3.6 | 5 (1× AG1856) | 4 | 8 |

1. A guide oligonucleotide capable of inducing editing of a nucleic acid encoding a human Proprotein Convertase Subtilisin/Kexin type 9 (PCSK9) proprotein, wherein the nucleic acid editing decreases or prevents the ability of the PCSK9 proprotein from being processed by auto-cleavage of a proteolytic cleavage site.

2. The guide oligonucleotide according to claim 1, wherein the proteolytic cleavage site is the P1 cleavage site.

3. The guide oligonucleotide according to claim 1, wherein the nucleic acid is RNA or DNA.

4. The guide oligonucleotide according to claim 3, wherein the nucleic acid is RNA and wherein the guide oligonucleotide can form a double stranded complex with a human PCSK9 pre-mRNA, mRNA, or a part thereof, wherein the guide oligonucleotide when complexed with the PCSK9 pre-mRNA, mRNA, or part thereof, is able to engage an endogenous human Adenosine Deaminases acting on RNA (ADAR) enzyme, thereby allowing the deamination of a target adenosine in the human PCSK9 pre-mRNA, mRNA, or part thereof.

5. The guide oligonucleotide according to claim 4, wherein the target adenosine is the second nucleotide of the codon coding for the glutamine residue at position 152 in the PCSK9 proprotein.

6. The guide oligonucleotide according to claim 4, wherein the guide oligonucleotide comprises an orphan nucleotide that is a cytidine, a cytidine analog, a uridine, or a uridine analog.

7. The guide oligonucleotide according to claim 6, wherein the orphan nucleotide is a cytidine analog comprising a m 6-amino-5-nitro-3-(1′-P-D-2′-deoxyribofuranosyl)-2(1H)-pyridone base.

8. The guide oligonucleotide according to claim 6, wherein the orphan nucleotide is a uridine analog and wherein the uridine analog is iso-uridine.

9. The guide oligonucleotide according to claim 4, wherein the guide oligonucleotide consists of 20 to 50 nucleotides, and wherein the guide oligonucleotide is substantially complementary to a region within the human PCSK9 pre-mRNA, mRNA, or part thereof that comprises the codon coding for the glutamine residue at position 152 in the PCSK9 proprotein.

10. The guide oligonucleotide according to claim 4, wherein the guide oligonucleotide is bound to a N-acetylgalactosamine (GalNAc) moiety.

11. The guide oligonucleotide according to claim 4, comprising one or more nucleotides that comprise a modification of a nucleobase, a sugar and/or an inter-nucleosidic linkage.

12. The guide oligonucleotide according to claim 11, wherein the guide oligonucleotide comprises:
(i) at least one non-naturally occurring inter-nucleosidic linkage modification selected from the group consisting of: phosphorothioate (PS), chirally pure PS, Rp PS, Sp PS, phosphorodithioate, phosphonoacetate, thiophosphonoacetate phosphonacetamide, thiophosphonacetamide, PS prodrug, S-alkylated PS, H-phosphonate, methyl phosphonate (MP), methyl phosphonothioate, methyl phosphate, methyl phosphorothioate, ethyl phosphate, ethyl PS, boranophosphate, boranophosphorothioate, methyl boranophosphate, methyl boranophosphorothioate, methyl boranophosphonate, methyl boranophosphonothioate, phosphorylguanidine, methyl sulfonylphosphoroamidate, phosphoramidite, phosphonamidite, N3′→P5′ phosphoramidate, N3′→P5′ thiophosphoramidate, phosphorodiamidate, phosphorothiodiamidate, sulfamate, dimethylenesulfoxide, sulfonate, triazole, oxalyl, carbamate, methyleneimino, thioacetamido, and their derivatives; and/or
ii at least one nucleotide that comprises a mono- or disubstitution at the 2′, 3′ and/or 5′ position of the sugar, selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; O-, S-, or N-alkyl; O-, S-, or N-alkenyl; O-, S-, or N-alkynyl; O-, S-, or N-allyl; O-alkyl-O-alkyl; -methoxy; -aminopropoxy; -methoxyethoxy; -dimethylamino oxyethoxy; and -dimethylaminoethoxyethoxy, with the proviso that the orphan base is not modified with 2′-O-methyl or 2′-methoxyethoxy.

13. A viral vector expressing a guide oligonucleotide of claim 1, wherein
(i) the guide oligonucleotide comprises an orphan nucleotide that is a cytidine or uridine,
(ii) the viral vector can treat, delay, or prevent cardiovascular disease, hypercholesterolemia, liver injury, and/or alcohol-induced steatohepatitis,
(iii) the oligonucleotide is capable of editing a nucleic acid encoding a human PCSK9 proprotein, and
(iv) the nucleic acid editing decreases or prevents the ability of the PCSK9 proprotein from being processed by auto-cleavage of a proteolytic cleavage site.

14. A pharmaceutical composition comprising the guide oligonucleotide of claim 1 or a viral vector comprising said guide oligonucleotide, and a pharmaceutically acceptable carrier.

15. A method for treating a human subject suffering from cardiovascular disease, hypercholesterolemia, liver injury, and/or alcohol-induced steatohepatitis, by decreasing or inhibiting an auto-cleaving ability of the PCSK9 proprotein in a liver cell of the subject, the method comprising administering to the subject the guide oligonucleotide of claim 1, or a viral vector or a pharmaceutical composition comprising said guide oligonucleotide, thereby editing a nucleic acid encoding the PCSK9 proprotein and thereby editing an auto-cleavage site of the PCSK9 proprotein.

16. The method of claim 15, wherein the auto-cleavage site is the P1 cleavage site of PCSK9 and the nucleic acid is RNA or DNA.

17. The method of claim 16, wherein
(i) the nucleic acid is RNA and the guide oligonucleotide can form a double stranded complex with a human PCSK9 pre-mRNA, mRNA, or a part thereof, and
(ii) the guide oligonucleotide is able to engage an endogenous human ADAR enzyme when complexed with the PCSK9 pre-mRNA, mRNA, or part thereof, thereby allowing the deamination of a target adenosine in the pre-mRNA, mRNA, or part thereof, at the P1 cleavage site.

18. The method of claim 17, wherein the adenosine in the codon encoding glutamine at position 152 of the PCSK9 proprotein is deaminated to an inosine.

19. The guide oligonucleotide according to claim 6, wherein the uridine analog is iso-uridine.

20. The guide oligonucleotide according to claim 4, wherein the guide oligonucleotide consists of 23 to 27 nucleotides, and wherein the guide oligonucleotide is substantially complementary to a region within the human PCSK9 pre-mRNA, mRNA, or part thereof that comprises the codon coding for the glutamine residue at position 152 in the PCSK9 proprotein.
